(19) United States
(12) Patent Application Publication
(10) Pub. No.: US 2023/0136301 A1
(43) Pub. Date: May 4, 2023

(54) Corticosteriod Reduction in Treatment with Anti-CD38 Antibody

(71) Applicant: Janssen Biotech, Inc., Horsham, PA (US)

(72) Inventors: Peter Hellemans, Beerse (BE); Ming Qi, Phoenixville, PA (US)

(21) Appl. No.: 17/979,874

(22) Filed: Nov. 3, 2022

Related U.S. Application Data
(60) Provisional application No. 63/275,157, filed on Nov. 3, 2021.
(60) Provisional application No. 63/280,791, filed on Nov. 18, 2021.
(60) Provisional application No. 63/288,785, filed on Dec. 13, 2021.
(60) Provisional application No. 63/394,726, filed on Aug. 3, 2022.

(51) Int. Cl. C07K 16/28; A61K 31/573; A61P 35/00

(52) U.S. Cl. CPC C07K 16/2896; A61K 31/573; A61P 35/00; A61K 2039/505

(57) ABSTRACT

This disclosure relates to methods of treating a hematologic malignancy, comprising administering to a subject a therapeutically effective amount of an anti-CD38 antibody and a corticosteroid for a time sufficient to treat the hematologic malignancy, wherein the dosing regimen includes a reduction, elimination, or reduction followed by elimination, of corticosteroid administration to the subject. This disclosure also relates to methods of treating a hematologic malignancy, comprising administering to a subject a therapeutically effective amount of an anti-CD38 antibody and a corticosteroid dose of <0.01 mg/kg/day or equivalent for a time sufficient to treat the hematologic malignancy. This disclosure also relates to methods of treating a hematologic malignancy, comprising administering to a subject a therapeutically effective amount of an anti-CD38 antibody for a time sufficient to treat the hematologic malignancy, wherein the dosing regimen results in a reduction, elimination or reduction and elimination of corticosteroid use by the subject.

### CROSS-REFRENCE TO RELATED APPLICATIONS

This application claims the benefit of U.S. Provisional Application Ser. No. 63/275,157, filed 3 Nov. 2021, Ser. No. 63/280,791, filed 22 Nov. 2021, Ser. No. 63/288,785, filed 20 Dec. 2021 and Ser. No. 63/394,726, filed 9 Aug. 2022. The entire contents of each of the aforementioned applications is incorporated herein by reference in their entirety.

### REFERENCE TO SEQUENCE LISTING SUBMITTED ELECTRONICALLY

This application contains a sequence listing, which is submitted electronically via The United States Patent and Trademark Center Patent Center as an XML formatted sequence listing with a file name “JBI6648USNP1 Sequence Listing.xml” and a creation date of Oct. 24, 2022, and having a size of 39 Kb. The sequence listing submitted via Patent Center is part of the specification and is herein incorporated by reference in its entirety.

### BACKGROUND

CD38 is a type II membrane protein having function in receptor-mediated adhesion and signaling as well as mediating calcium mobilization via its ecto-enzymatic activity, catalyzing formation of cyclic ADP-ribose (cADPR) from NAD⁺ and also hydrolyzing cADPR into ADP-ribose (ADPR). CD38 mediates cytokine secretion and activation and proliferation of lymphocytes (Funaro et al., J Immunol. 145(8): 2390-96 (1990); Terhorst et al., Cell 23(3): 771-80 (1981); Guse et al., Nature 398: 70-73 (1999)), and via its NAD glycohydrolase activity regulates extracellular NAD⁺ levels which have been implicated in modulating the regulatory T-cell compartment (Adriouch et al., Microbes Infect. 14(14):1284-92 (2012) and Chiarugi et al., Nat Rev Cancer. 12(11):741-52 (2012)).

CD38 is expressed in a number of hematologic malignancies including B-cell acute lymphoblastic leukemia (ALL), B-cell chronic lymphocytic leukemia, B-cell non-Hodgkin lymphoma, multiple myeloma, and T-cell ALL. CD38 is also expressed in B-cell disorders such as light chain amyloidosis, monoclonal gammopathy of undetermined significance (MGUS) and smoldering multiple myeloma (SMM).

B-cell malignancies include B-cell chronic lymphocytic leukemia, mantle cell lymphoma, Burkitt lymphoma, follicular lymphoma, diffuse large B-cell lymphoma, multiple myeloma, Hodgkin's lymphoma, hairy cell leukemia, primary effusion lymphoma and AIDS-related Non-Hodgkin's Lymphoma. B-cell malignancies comprise more than 85% of diagnosed lymphomas.

Multiple myeloma (MM) is a B cell malignancy characterized by the latent accumulation of secretory plasma cells in bone marrow with a low proliferative index and an extended life span. The disease ultimately attacks bones and bone marrow, resulting in multiple tumors and lesions throughout the skeletal system. Approximately 1% of all cancers, and slightly more than 10% of all hematologic malignancies, can be attributed to MM. Incidence of MM increases in the aging population, with the median age at time of diagnosis being about 61 years.

### SUMMARY

There is a need for additional anti-CD38 antibody based therapies for the treatment of hematologic malignancies such as MM and other B-cell malignancies.

The disclosure generally relates to methods that are useful for treating hematologic malignancies (e.g., hematologic cancers such as multiple myeloma).

In one aspect, the disclosure provides a method of treating a hematologic malignancy, comprising administering to a subject in need thereof a therapeutically effective amount of an anti-CD38 antibody and a corticosteroid for a time sufficient to treat the hematologic malignancy, wherein the dosing regimen includes a reduction, elimination, or reduction followed by elimination, of corticosteroid administration to the subject.

In some embodiments, the corticosteroid comprises bethamethasone, cortisol, cortisone, dexamethasone, glucocorticoid, hydrocortisone, methylprednisolone (MP), prednisolone, prednisone, triamcinolone, or a combination thereof. In some embodiments, the corticosteroid comprises MP, dexamethasone, prednisone, or a combination thereof.

In some embodiments, the corticosteroid administered to the subject is reduced by about 60% and then eliminated during a 28-day treatment cycle.

In some embodiments, the corticosteroid administered to the subject is reduced by about 60% and then by about 30% and then eliminated during a 28-day treatment cycle.

In some embodiments, the corticosteroid administered to the subject is administered once and then eliminated during a 28-day treatment cycle.

In certain embodiments, the anti-CD38 antibody is administered once weekly, every 2 weeks, or every 4 weeks during a 28-day cycle. In particular embodiments, the anti-CD38 antibody is administered once weekly during Cycle 1, every 2 weeks during Cycles 2-5, and every 4 weeks thereafter.

In some embodiments, the method comprises:
a) administering about 1,800 mg daratumumab and about 30,000 U rHuPH20 recombinant hyaluronidase subcutaneously on days 1, 8, 15 and 22; and
b) administering about 20 mg pre-dose corticosteroid (e.g., dexamethasone) intravenously on day 1, of the 28-day cycle.

In some embodiments, the method comprises:

administering about 1,800 mg daratumumab and about 30,000 U rHuPH20 recombinant hyaluronidase subcutaneously on days 1, 8, 15 and 22;

administering about 100 mg pre-dose corticosteroid (e.g., MP) orally or intravenously on day 1;

administering about 20 mg post-dose corticosteroid (e.g., MP) orally on days 1 and 2;

administering about 60 mg pre-dose corticosteroid (e.g., MP) orally or intravenously on day 8; and

administering about 20 mg post-dose corticosteroid (e.g., MP) orally on day 8, of the 28-day cycle.

In some embodiments, the method comprises:

administering about 1,800 mg daratumumab and about 30,000 U rHuPH20 recombinant hyaluronidase subcutaneously on days 1, 8, 15 and 22;

administering about 100 mg pre-dose corticosteroid (e.g., MP) orally or intravenously on day 1;

administering about 20 mg post-dose corticosteroid (e.g., MP) orally on days 1 and 2;

administering about 60 mg pre-dose corticosteroid (e.g., MP) orally or intravenously on day 8;

administering about 20 mg post-dose corticosteroid (e.g., MP) orally on day 8;

administering about 30 mg pre-dose corticosteroid (e.g., MP) orally or intravenously on day 15; and

administering about 20 mg post-dose corticosteroid (e.g., MP) orally on day 15, of the 28-day cycle.

In another aspect, the disclosure provides a method of treating hematologic cancer to a subject in need thereof, comprising administering to the subject a therapy on a 28-day cycle, wherein the therapy comprises:

administering about 1,800 mg anti-CD38 antibody and about 30,000 U hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8;

administering about 20 mg post-dose corticosteroid on day 8;

administering about 30 mg pre-dose corticosteroid on day 15; and

administering about 20 mg post-dose corticosteroid on day 15, of the 28-day cycle.

In another aspect, the disclosure provides a method of treating hematologic cancer to a subject in need thereof, comprising administering to the subject a therapy on a 28-day cycle, wherein the therapy comprises:

administering about 1,800 mg anti-CD38 antibody and about 30,000 U rHuPH20 hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8; and

administering about 20 mg post-dose corticosteroid on day 8, of the 28-day cycle.

In another aspect, the disclosure provides a method of treating hematologic cancer in a subject in need thereof, the method comprising administering to the subject a therapy on a 28-day cycle, wherein the therapy comprises:
a) administering about 1,800 mg of the anti-CD38 antibody and about 30,000 U on days 1, 8, 15 and 22; and
b) administering about 20 mg pre-dose corticosteroid on day 1, of the 28-day cycle.

In another aspect, the disclosure provides a method of treating a hematologic malignancy, wherein the method comprises administering to a subject in need thereof a therapeutically effective amount of an anti-CD38 antibody for a time sufficient to treat the hematologic malignancy, wherein the dosing regimen results in a reduction, elimination or reduction and elimination of corticosteroid use by the subject.

In another aspect, the disclosure provides a method of treating a hematologic malignancy, wherein the method comprises administering to a subject in need thereof a therapeutically effective amount of an anti-CD38 antibody and a corticosteroid dose of <2 mg/kg/day or equivalent for a time sufficient to treat the hematologic malignancy. In some embodiments, a corticosteroid dose of <0.05 mg/kg/day or equivalent is administered.

In another aspect, the disclosure provides a method of treating a hematologic malignancy, wherein the method comprises administering to a subject in need thereof a therapeutically effective amount of an anti-CD38 antibody without co-administering a corticosteroid for a time sufficient to treat the hematologic malignancy.

In another aspect, the disclosure provides a method of treating a hematologic malignancy, wherein the method comprises administering to a subject in need thereof a therapeutically effective amount of an anti-CD38 antibody for a time sufficient to treat the hematologic malignancy, wherein disease control or complete remission is achieved and/or maintained at a corticosteroid dose of <2 mg/kg/day or equivalent. In some embodiments, disease control or complete remission is achieved and/or maintained at a corticosteroid dose of <0.05 mg/kg/day or equivalent.

In some embodiments, the method further comprises administering to the subject a prior therapy on a 28-day cycle, comprising:
a) administering about 1,800 mg daratumumab and about 30,000 U rHuPH20 recombinant hyaluronidase subcutaneously on days 1, 8, 15 and 22; and
b) administering about 20 mg pre-dose corticosteroid (e.g., dexamethasone) intravenously on day 1, of the 28-day cycle.

In some embodiments, the method further comprises administering to the subject a prior therapy on a 28-day cycle, comprising:

administering about 1,800 mg daratumumab and about 30,000 U rHuPH20 recombinant hyaluronidase subcutaneously on days 1, 8, 15 and 22;

administering about 100 mg pre-dose corticosteroid (e.g., MP) orally or intravenously on day 1;

administering about 20 mg post-dose corticosteroid (e.g., MP) orally on days 1 and 2;

administering about 60 mg pre-dose corticosteroid (e.g., MP) orally or intravenously on day 8; and

administering about 20 mg post-dose corticosteroid (e.g., MP) orally on day 8, of the 28-day cycle.

In some embodiments, the method comprises administering to the subject a prior therapy on a 28-day cycle, comprising:

administering about 1,800 mg daratumumab and about 30,000 U rHuPH20 recombinant hyaluronidase subcutaneously on days 1, 8, 15 and 22;

administering about 100 mg pre-dose corticosteroid (e.g., MP) orally or intravenously on day 1;

administering about 20 mg post-dose corticosteroid (e.g., MP) orally on days 1 and 2;

administering about 60 mg pre-dose corticosteroid (e.g., MP) orally or intravenously on day 8;

administering about 20 mg post-dose corticosteroid (e.g., MP) orally on day 8;

administering about 30 mg pre-dose corticosteroid (e.g., MP) orally or intravenously on day 15; and

administering about 20 mg post-dose corticosteroid (e.g., MP) orally on day 15, of the 28-day cycle.

In some embodiments, the hematologic malignancy is a CD38-positive hematologic malignancy. In certain embodiments, the CD38-positive hematologic malignancy is multiple myeloma. In particular embodiments, the multiple myeloma is relapsed or refractory multiple myeloma.

In some embodiments, the anti-CD38 antibody comprises:
a) a heavy chain complementarity determining region 1 (HCDR1), HCDR2 and HCDR3 amino acid sequences of SEQ ID NOs:6, 7 and 8, respectively; and/or
b) a light chain complementarity determining region 1 (LCDR1), LCDR2 and LCDR3 amino acid sequences of SEQ ID NOs:9, 10 and 11, respectively.

In certain embodiments, the anti-CD38 antibody comprises a heavy chain variable region (VH) sequence of SEQ ID NO:4, a light chain variable region (VL) sequence of SEQ ID NO:5, or both. In particular embodiments, the anti-CD38 antibody comprises a heavy chain sequence of SEQ ID NO:12, a light chain sequence of SEQ ID NO:13, or both.

In some embodiments, the anti-CD38 antibody is of the IgG1, IgG2, IgG3 or IgG4 subtype. In certain embodiments, the anti-CD38 antibody is of the IgG1 subtype. In particular embodiments, the anti-CD38 antibody is of the IgG1/κ subtype. In some embodiments, the anti-CD38 antibody is daratumumab.

In certain embodiments, the pharmaceutical composition further comprises a hyaluronidase. In particular embodiments, the hyaluronidase is rHuPH20 recombinant hyaluronidase.

In some embodiments, the anti-CD38 antibody is administered in a pharmaceutical composition comprising from about 1,200 mg to about 5,000 mg of the anti-CD38 antibody. In some embodiments, the pharmaceutical composition comprises about 1,800 mg of the anti-CD38 antibody.

In some embodiments, the pharmaceutical composition further comprises a hyaluronidase. In some embodiments, the hyaluronidase is rHuPH20 recombinant hyaluronidase. In some embodiments, the pharmaceutical composition comprises from about 750 U to about 75,000 U of the hyaluronidase. In some embodiments, the pharmaceutical composition comprises about 30,000 U of the hyaluronidase.

In some embodiments, the anti-CD38 antibody and the hyaluronidase are administered in a co-formulation.

In some embodiments, the anti-CD38 antibody is administered in a pharmaceutical composition comprising about 1,800 mg of the anti-CD38 antibody and about 30,000 U of the hyaluronidase.

In some embodiments, the pharmaceutical composition further comprises:

about 4.9 mg L-histidine;

about 18.4 mg L-histidine hydrochloride monohydrate;

about 13.5 mg L-methionine;

about 6 mg polysorbate 20 (PS-20); and

about 735.1 mg sorbitol.

In some embodiments, the pharmaceutical composition has a pH of about pH 5.5. In other embodiments, the pharmaceutical composition has a pH of about pH 5.6.

In some embodiments, the pharmaceutical composition has a total volume of about 15 mL.

In some embodiments, the anti-CD38 antibody is administered subcutaneously.

In some embodiments, the subject is 18 years of age or older.

In some embodiments, the subject is naïve to anti-CD38 therapy.

In some embodiments, the subject has received at least two prior lines of anti-myeloma therapy. In certain embodiments, the at least two prior lines of anti-myeloma therapy comprise a proteasome inhibitor (PI), administering an immunomodulatory drug (IMiD), hematopoietic stem cell transplantation (HSCT), a maintenance therapy, or a combination thereof. In particular embodiments, the IMid is lenalidomide. In some embodiments, the PI is bortezomib, carfilzomib, or ixazomib. In certain embodiments, the HSCT is an autologous HSCT. the two lines of therapy comprise am IMid and a PI.

In some embodiments, the subject is refractory to at least one line of therapy.

In some embodiments, the method elicits at least a partial response in the subject. In certain embodiments, a partial response in the subject.

In some embodiments, the method elicits at least a very good partial response in the subject. In certain embodiments, elicits a complete response in the subject. In particular embodiments, the method elicits a stringent complete response in the subject.

In some embodiments, the method improves one or more outcome measurements of the subject. In some embodiments, the one or more outcome measurements comprise progression-free survival, duration of response, or at least partial response, or any combination thereof. In some embodiments, the one or more outcome measures comprise a partial response, a very good partial response, a complete response, or a stringent complete response.

In some embodiments, the subject experiences an improvement in one or more outcome measures consistent with a subject receiving anti-CD38 antibody administration and continuous corticosteroid administration. In other words, the difference in improvement in the subject treated with a method recited herein and a subject treated without a reduction or elimination of corticosteroid administration is not (statistically) significant.

In some embodiments, the subject experiences an increased improvement in one or more outcome measures compared with a subject receiving anti-CD38 antibody administration and continuous corticosteroid administration. In other words, there is an improvement in the subject treated with a method recited herein and a subject treated without a reduction or elimination of corticosteroid administration.

In some embodiments, the method further comprises administering to the subject one or more additional therapeutic agents. In certain embodiment, the one or more additional therapeutic agents comprise a T cell expressing chimeric antigen receptor (CAR) (CAR-T cell), a natural killer cell expressing CAR (CAR-NK cell), a macrophage expressing CAR (CAR-M cell), a chemotherapeutic agent, a bispecific antibody, an immune checkpoint inhibitor, or a combination thereof.

In some embodiments, the CAR-T cell (CART cell), the CAR-NK cell, or the CAR-M cell is allogeneic. In some embodiments, the CAR comprises an extracellular antigen-binding domain, a transmembrane domain and an intracellular signaling domain, and wherein the intracellular signaling domain comprises a T-cell surface glycoprotein CD3 zeta chain component.

In some embodiments, the extracellular antigen-binding domain binds an G-protein coupled receptor family C group 5 member D (GPRC5D) antigen. In certain embodiments, the extracellular antigen-binding domain binds GPRC5D and CD3. In particular embodiments, the one or more additional therapeutic agents comprise an anti-GPRC5D CAR-T and an anti-GPRC5D CAR-NK.

In some embodiments, the extracellular antigen-binding domain binds an B cell maturation antigen (BCMA) antigen. In certain embodiments, the extracellular antigen-binding domain binds BCMA and CD3. In particular embodiments, the one or more additional therapeutic agents comprise an anti-BCMA CAR-T and an anti-BCMA CAR-NK.

In particular embodiments, the immune checkpoint inhibitor comprises an anti-PD-1 antibody, an anti-PD-L1 antibody, an anti-PD-L2 antibody, an anti-LAG3 antibody, an anti-TIM3 antibody, an anti-CTLA-4 antibody, or a combination thereof.

In some embodiments, the T-cell redirector comprises a soluble bispecific antibody (bsAb) or a membrane-anchored chimeric antigen receptor, or a combination thereof. In some embodiments, the bispecific antibody binds GPRC5D. In certain embodiments, the bispecific antibody binds GPRC5D and CD3. In some embodiments, the bispecific antibody binds BCMA. In certain embodiments, the bispecific antibody binds BCMA and CD3.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 depicts the PAVO Part 3 study design. Patients received either a 3-week tapering schedule, a 2-week tapering schedule, or a 1-week tapering schedule. RRMM, relapsed or refractory multiple myeloma; DARA SC, daratumumab subcutaneous; rHuPH20, recombinant human hyaluronidase PH20; IV, intravenous; ORR, overall response rate; CR, complete response. Pre-/post-administration medication included acetaminophen, diphenhydramine, montelukast, and methylprednisolone. Weekly in Cycles 1 and 2, every 2 weeks in Cycles 3-6, and every 4 weeks thereafter.

FIGS. 2A-2C depict the corticosteroid-tapering schedules for each cohort. Patients in the 3-week tapering cohort were corticosteroid-free by Cycle 1 Day 22 (FIG. 2A), patients in the 2-week tapering cohort were corticosteroid-free by Cycle 1 Day 15 (FIG. 2B), and patients in the 1-week tapering group were corticosteroid-free by Cycle 1 Day 8 (FIG. 2C). C, cycle; CS, corticosteroid; D, day; DEX, dexamethasone; DLT, dose-limiting toxicity; IV, intravenous; MP, methylprednisolone; PO, orally.

FIG. 3 shows serum daratumumab concentrations (μg/mL) over time (from baseline to Cycle 1 Day 22). Box plot of serum daratumumab concentrations over time in the pharmacokinetic evaluable population.

FIG. 4 shows serum daratumumab concentrations (μg/mL) over time (from Cycle 2 Day 1 onwards). Box plot of serum daratumumab concentrations over time in the pharmacokinetic-evaluable population.

FIG. 5 shows the response rates in the total all-treated patient population. PR, partial response; VGPR, very good partial response; CR, complete response; ORR, overall response rate.

### DETAILED DESCRIPTION

A description of example embodiments follows.

While example embodiments have been particularly shown and described, it will be understood by those skilled in the art that various changes in form and details may be made therein without departing from the scope of the embodiments encompassed by the appended claims.

Various publications, articles and patents are cited or described in the background and throughout the specification; each of these references is herein incorporated by reference in its entirety. Discussion of documents, acts, materials, devices, articles or the like, which has been included in the present specification, is for the purpose of providing context to the present invention. Such discussion is not an admission that any or all of these matters form part of the prior art with respect to any inventions disclosed or claimed.

The disclosure is based on, at least in part, the discovery that daratumumab treatment permits quick steroid tapering in relapsed or refractory multiple myeloma patients.

“Relapse” refers to progression of disease after an initial response to previous treatment, more than 60 days after cessation of treatment. “Refractory disease” refers to less than (<) 25 percent (%) reduction in M-protein or progression of disease during treatment or within 60 days after cessation of treatment.

In one aspect, the disclosure provides a method of treating a hematologic malignancy, comprising administering to a subject in need thereof a therapeutically effective amount of an anti-CD38 antibody and a corticosteroid for a time sufficient to treat the hematologic malignancy, wherein the dosing regimen includes a reduction, elimination, or reduction followed by elimination, of corticosteroid administration to the patient.

In another aspect, the disclosure provides a method of treating a hematologic malignancy, wherein the method comprises administering to a subject in need thereof a therapeutically effective amount of an anti-CD38 antibody for a time sufficient to treat the hematologic malignancy, wherein the dosing regimen results in a reduction, elimination or reduction and elimination of corticosteroid use by the patient.

In another aspect, the disclosure provides a method of treating a hematologic malignancy, wherein the method comprises administering to a subject in need thereof a therapeutically effective amount of an anti-CD38 antibody and a corticosteroid dose of <2 mg/kg/day or equivalent for a time sufficient to treat the hematologic malignancy.

In another aspect, the disclosure provides a method of treating a hematologic malignancy, wherein the method comprises administering to a subject in need thereof a therapeutically effective amount of an anti-CD38 antibody without co-administering a corticosteroid for a time sufficient to treat the hematologic malignancy.

In another aspect, the disclosure provides a method of treating a hematologic malignancy, wherein the method comprises administering to a subject in need thereof a therapeutically effective amount of an anti-CD38 antibody for a time sufficient to treat the hematologic malignancy, wherein disease control or complete remission is achieved and/or maintained at a corticosteroid dose of <2 mg/kg/day or equivalent.

### Anti-CD38 Antibodies

The term “CD38” refers to the CD38 protein (synonyms include: ADP-ribosyl cyclase 1, cADPr hydrolase 1, cyclic ADP-ribose hydrolase 1). In some embodiments, CD38 is human CD38 (SEQ ID NO:1). Human CD38 is a single-pass type II membrane protein with amino acid residues 1-21 representing the cytosolic domain, amino acid residues 22-42 representing the transmembrane domain, and amino acid residues 43-300 representing the extracellular domain. Human CD38 has an amino acid sequence shown in GenBank accession number NP_001766. The amino acid sequence of SEQ ID NOs:1-40 are provided in Table 1.

TABLE 1
Amino Acid Sequences

| SEQ ID | |
|---|---|
| NO: | Amino Acid Sequences |
| 1 | MANCEFSPVSGDKPCCRLSRRAQLCLGVSILVLILVVVLAVVVPRWRQQWSGPGTTKRFPET |
| | VLARCVKYTEIHPEMRHVDCQSVWDAFKGAFISKHPCNITEEDYQPLMKLGTQTVPCNKILL |
| | WSRIKDLAHQFTQVQRDMFTLEDTLLGYLADDLTWCGEFNTSKINYQSCPDWRKDCSNNPVS |
| | VFWKTVSRRFAEAACDVVHVMLNGSRSKIFDKNSTFGSVEVHNLQPEKVQTLEAWVIHGGRE |
| | DSRDLCQDPTIKELESIISKRNIQFSCKNIYRPDKFLQCVKNPEDSSCTSEI |
| 2 | SKRNIQFSCKNIYR |
| 3 | EKVQTLEAWVIHGG |
| 4 | EVQLLESGGGLVQPGGSLRLSCAVSGFTFNSFAMSWVRQAPGKGLEWVSAISGSGGGTYYAD |
| | SVKGRFTISRDNSKNTLYLQMNSLRAEDTAVYFCAKDKILWFGEPVFDYWGQGTLVTVSS |
| 5 | EIVLTQSPATLSLSPGERATLSCRASQSVSSYLAWYQQKPGQAPRLLIYDASNRATGIPARF |
| | SGSGSGTDFTLTISSLEPEDFAVYYCQQRSNWPPTFGQGTKVEIK |
| 6 | SFAMS |
| 7 | AISGSGGGTYYADSVKG |
| 8 | DKILWFGEPVFDY |
| 9 | RASQSVSSYLA |
| 10 | DASNRAT |
| 11 | QQRSNWPPTF |
| 12 | EVQLLESGGGLVQPGGSLRLSCAVSGFTFNSFAMSWVRQAPGKGLEWVSAISGSGGGTYYAD |
| | SVKGRFTISRDNSKNTLYLQMNSLRAEDTAVYFCAKDKILWFGEPVFDYWGQGTLVTVSSAS |
| | TKGPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCPAPELLGGPSVFLF |
| | PPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGNVFSCSVMH |
| | EALHNHYTQKSLSLSPGK |
| 13 | EIVLTQSPATLSLSPGERATLSCRASQSVSSYLAWYQQKPGQAPRLLIYDASNRATGIPARF |
| | SGSGSGTDFTLTISSLEPEDFAVYYCQQRSNWPPTFGQGTKVEIKRTVAAPSVFIFPPSDEQ |
| | LKSGTASVVCLLNNFYPREAKVQWKVDNALQSGNSQESVTEQDSKDSTYSLSSTLTLSKADY |
| | EKHKVYACEVTHQGLSSPVTKSFNRGEC |
| 14 | QVQLVQSGAEVKKPGSSVKVSCKASGGTFSSYAFSWVRQAPGQGLEWMGRVIPFLGIANSAQ |
| | KFQGRVTITADKSTSTAYMDLSSLRSEDTAVYYCARDDIAALGPFDYWGQGTLVTVSSAS |
| 15 | DIQMTQSPSSLSASVGDRVTITCRASQGISSWLAWYQQKPEKAPKSLIYAASSLQSGVPSRF |
| | SGSGSGTDFTLTISSLQPEDFATYYCQQYNSYPRTFGQGTKVEIK |
| 16 | EVQLVQSGAEVKKPGESLKISCKGSGYSFSNYWIGWVRQMPGKGLEWMGIIYPHDSDARYSP |
| | SFQGQVTFSADKSISTAYLQWSSLKASDTAMYYCARHVGWGSRYWYFDLWGRGTLVTVSS |
| 17 | EIVLTQSPATLSLSPGERATLSCRASQSVSSYLAWYQQKPGQAPGLLIYDASNRASGIPARF |
| | SGSGSGTDFTLTISSLEPEDFAVYYCQQRSNWPLTFGGGTKVEIK |
| 18 | QVQLVESGGGLVQPGGSLRLSCAASGFTFSSYYMNWVRQAPGKGLEWVSGISGDPSNTYYAD |
| | SVKGRFTISRDNSKNTLYLQMNSLRAEDTAVYYCARDLPLVYTGFAYWGQGTLVTVSS |
| 19 | DIELTQPPSVSVAPGQTARISCSGDNLRHYYVYWYQQKPGQAPVLVIYGDSKRPSGIPERFS |
| | GSNSGNTATLTISGTQAEDEADYYCQTYTGGASLVFGGGTKLTVLGQ |
| 20 | QVQLVQSGAEVAKPGTSVKLSCKASGYTFTDYWMQWVKQRPGQGLEWIGTIYPGDGDTGYAQ |
| | KFQGKATLTADKSSKTVYMHLSSLASEDSAVYYCARGDYYGSNSLDYWGQGTSVTVSS |
| 21 | DIVMTQSHLSMSTSLGDPVSITCKASQDVSTVVAWYQQKPGQSPRRLIYSASYRYIGVPDRF |
| | TGSGAGTDFTFTISSVQAEDLAVYYCQQHYSPPYTFGGGTKLEIK |
| 22 | LNFRAPPVIPNVPFLWAWNAPSEFCLGKFDEPLDMSLFSFIGSPRINATGQGVTIFYVDRLG |
| | YYPYIDSITGVTVNGGIPQKISLQDHLDKAKKDITFYMPVDNLGMAVIDWEEWRPTWARNWK |
| | PKDVYKNRSIELVQQQNVQLSLTEATEKAKQEFEKAGKDFLVETIKLGKLLRPNHLWGYYLF |
| | PDCYNHHYKKPGYNGSCFNVEIKRNDDLSWLWNESTALYPSIYLNTQQSPVAATLYVRNRVR |
| | EAIRVSKIPDAKSPLPVFAYTRIVFTDQVLKFLSQDELVYTFGETVALGASGIVIWGTLSIM |
| | RSMKSCLLLDNYMETILNPYIINVTLAAKMCSQVLCQEQGVCIRKNWNSSDYLHLNPDNFAI |
| | QLEKGGKFTVRGKPTLEDLEQFSEKFYCSCYSTLSCKEKADVKDTDAVDVCIADGVCIDAFL |
| | KPPMETEEPQIFY |
| 23 | QVQLVQSGVEVKKPGASVKVSCKASGYTFTNYYMYWVRQAPGQGLEWMGGINPSNGGTNFNE |
| | KFKNRVTLTTDSSTTTAYMELKSLQFDDTAVYYCARRDYRFDMGFDYWGQGTTVTVSS |
| 24 | EIVLTQSPATLSLSPGERATLSCRASKGVSTSGYSYLHWYQQKPGQAPRLLIYLASYLESGV |
| | PARFSGSGSGTDFTLTISSLEPEDFAVYYCQHSRDLPLTFGGGTKVEIK |
| 25 | QVQLVESGGGVVQPGRSLRLDCKASGITFSNSGMHWVRQAPGKGLEWVAVIWYDGSKRYYAD |
| | SVKGRFTISRDNSKNTLFLQMNSLRAEDTAVYYCATNDDYWGQGTLVTVSS |
| 26 | EIVLTQSPATLSLSPGERATLSCRASQSVSSYLAWYQQKPGQAPRLLIYDASNRATGIPARF |
| | SGSGSGTDFTLTISSLEPEDFAVYYCQQSSNWPRTFGQGTKVEIK |
| 27 | EVQLVESGGGLVQPGGSLRLSCAASGFTFSRYWMSWVRQAPGKGLEWVANIKQDGSEKYYVD |
| | SVKGRFTISRDNAKNSLYLQMNSLRAEDTAVYYCAREGGWFGELAFDYWGQGTLVTVSS |
| 28 | EIVLTQSPGTLSLSPGERATLSCRASQRVSSSYLAWYQQKPGQAPRLLIYDASSRATGIPDR |
| | FSGSGSGTDFTLTISRLEPEDFAVYYCQQYGSLPWTFGQGTKVEIK |
| 29 | EVQLVESGGGLVQPGGSLRLSCAASGFTFSDSWIHWVRQAPGKGLEWVAWISPYGGSTYYAD |
| | SVKGRFTISADTSKNTAYLQMNSLRAEDTAVYYCARRHWPGGFDYWGQGTLVTVSS |
| 30 | DIQMTQSPSSLSASVGDRVTITCRASQDVSTAVAWYQQKPGKAPKLLIYSASFLYSGVPSRF |
| | SGSGSGTDFTLTISSLQPEDFATYYCQQYLYHPATFGQGTKVEIK |
| 31 | EVQLLESGGGLVQPGGSLRLSCAASGFTFSSYIMMWVRQAPGKGLEWVSSIYPSGGITFYAD |
| | TVKGRFTISRDNSKNTLYLQMNSLRAEDTAVYYCARIKLGTVTTVDYWGQGTLVTVSS |
| 32 | QSALTQPASVSGSPGQSITISCTGTSSDVGGYNYVSWYQQHPGKAPKLMIYDVSNRPSGVSN |
| | RFSGSKSGNTASLTISGLQAEDEADYYCSSYTSSSTRVFGTGTKVTVL |
| 33 | QVQLVQSGAEVKKPGSSVKVSCKASGGTFSSYAISWVRQAPGQGLEWMGGIIPIFDTANYAQ |
| | KFQGRVTITADESTSTAYMELSSLRSEDTAVYYCARPGLAAAYDTGSLDYWGQGTLVTVSS |
| 34 | EIVLTQSPATLSLSPGERATLSCRASQSVRSYLAWYQQKPGQAPRLLIYDASNRATGIPARF |
| | SGSGSGTDFTLTISSLEPEDFAVYYCQQRNYWPLTFGQGTKVEIK |
| 35 | EVQLVESGGGLVQPGGSLRLSCAASGFAFSRYDMSWVRQAPGKGLESVAYISGGGANTYYLD |
| | NVKGRFTISRDNAKNSLYLQMNSLRAEDTAVYYCASPYLSYFDVWGQGTLVTVSS |
| 36 | EIVMTQSPATLSVSPGERATLSCRASQSLSDYLHWYQQKPGQAPRLLIKSASQSISGIPARF |
| | SGSGSGTEFTLTISSLQSEDFAVYYCQNGHSFPYTFGQGTKLEIK |
| 37 | EVQLLESGGGLVQPGGSLRLSCAASGFTFSSYAMSWVRQAPGKGLEWVSAISGSGGSTYYAD |
| | SVKGRFTISRDNSKNTLYLQMNSLRAEDTAVYYCAKSPYAPLDYWGQGTLVTVSS |
| 38 | EIVLTQSPATLSLSPGERATLSCRASQSVNDYLAWYQQKPGQAPRLLIYDASNRATGIPARF |
| | SGSGSGTDFTLTISSLEPEDFAVYYCQQGGHAPITFGQGTKVEIK |
| 39 | EVQLVQSGAEVKKPGESLKISCKGSGYSFTSYWMQWVRQMPGKGLEWMGAIYPGDGDIRYTQ |
| | NFKGQVTISADKSISTAYLQWSSLKASDTAMYYCARWEKSTTVVQRNYFDYWGQGTTVTVSS |
| 40 | DIQMTQSPSSLSASVGDRVTITCKASENVGTFVSWYQQKPGKAPKLLIYGASNRYTGVPSRF |
| | SGSGSGTDFTLTISSLQPEDFATYYCGQSYSYPTFGQGTKLEIK |

In some embodiments, an anti-CD38 antibody of the present disclosure binds human CD38 (SEQ ID NO:1). In some embodiments, an anti-CD38 antibody is specific for a human CD38 epitope. “Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come into close proximity in a three-dimensional space through the folding of the protein molecule. In certain embodiments, the anti-CD38 antibody binds at least to the region SKRNIQFSCKNIYR (SEQ ID NO:2) and the region EKVQTLEAWVIHGG (SEQ ID NO:3) of human CD38 (SEQ ID NO:1). Antibodies binding to the region having the sequence of SKRNIQFSCKNIYR (SEQ ID NO: 2) and the region having the sequence of EKVQTLEAWVIHGG (SEQ ID NO: 3) of human CD38 (SEQ ID NO: 1) may be generated, for example, by immunizing mice with peptides having the amino acid sequences shown in SEQ ID NOs: 2 and 3 using standard methods and those described herein, and characterizing the obtained antibodies for binding to the peptides using for example ELISA or mutagenesis studies.

As used herein, the term “anti-CD38 antibody” refers to an immunoglobulin molecule capable of specific binding to CD38 through at least one antigen recognition site, located in the variable region of the immunoglobulin molecule. Typically, the antibody binds to CD38 with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with a K_{D} that is at least one hundred-fold less than its K_{D} for binding to a non-specific antigen (e.g., BSA, casein). The K_{D} may be measured using standard procedures. Antibodies that specifically bind CD38 may, however, have cross-reactivity to other related antigens, for example to the same antigen from other species (homologs), such as monkey, for example Macaca fascicularis (cynomolgus, cyno), Pan troglodytes (chimpanzee, chimp) or Callithrix jacchus (common marmoset, marmoset). In one embodiment, the anti-CD38 antibody binds to an epitope on human CD38 that includes amino acid residues 233-246 and 267-280 of CD38.

As used herein, the term “antibody” refers to a full-length antibody or an antigen-binding fragment of a full-length antibody.

A full-length antibody comprises two heavy (H) chains and two light (L) chains inter-connected by disulfide bonds or multimers thereof (e.g., IgM). Each heavy chain comprises a heavy chain variable region (V_{H}) and a heavy chain constant region (comprising domains CH1, hinge CH2 and CH3). Each light chain comprises a light chain variable region (V_{L}) and a light chain constant region (CL). The V_{H} and the V_{L} regions may be further subdivided into regions of hypervariability, termed complementarity determining regions (CDRs), interspersed within framework regions (FR). V_{H} and V_{L} each comprises three CDRs and four FR segments, arranged from the amino-terminus to the carboxy-terminus in the following order: FR1, CDR1, FR2, CDR2, FR3, CDR3, and FR4.

“Complementarity determining regions (CDRs)” are “antigen binding sites” in an antibody. “CDR” encompasses any CDR defined by an art-recognized method for identifying the CDR residues of an antibody, for example, by Kabat (HCDR1, HCDR2, HCDR3, LCDR1, LCDR2 and LCDR3, based on sequence variability, Wu & Kabat, J Exp Med 132:211-50 (1970), Kabat et al., Sequences of Proteins of Immunological Interest, 5^{th} edition, U.S. Department of Health and Human Services, NIH Publication No. 91-3242 (1991)), by Chothia (“Hypervariable regions” (HVR or HV) H1, H2, H3, L1, L2 and L3, Chothia & Lesk, Mol Biol 196:901-17 (1987), Chothia et al., (1989) Nature 342:877)), by the International ImMunoGeneTics (IMGT) database (a standardized numbering and definition of antigen-binding sites, www_imgt_org), by the AbM definition, or by the contact definition. The correspondence between CDRs, HVs and IMGT delineations is described in Lefranc et al., Dev. Comparat. Immunol. 27:55-77 (2003). Also see Al-lazikani et al., J Molec Biol 273:927-48 (1997), Almagro, J Mol Recognit 17:132-43 (2004), and hgmp.mrc.ac.uk and bioinf.org.uk/abs. Publicly and/or commercially available tools for identifying framework and/or CDR regions include, IgBlast (www_ncbi_nlm_nih_gov/igblast/), Scaligner (drugdesigntech at www_scaligner_com/), IMGT rules and/or tools (www_imgt_org/IMGTScientificChartNomenclature/IMGT-FRCDRdefinition.html), Chothia canonical assignment (www_bioinf_org_uk/abs/Chothia_html), Antigen receptor Numbering And Receptor Calssificatilon (ANARCI, opig.stats.ox.ac.uk/webapps/newsabdab/sabpred/anarci/), or the Paratome web server (www_ofranlab_org/paratome/, see Kunik et al, Nucleic Acids Research 40(W1):W521-W524 (2012)). Unless explicitly stated otherwise, the term “CDR”, “HCDR1”, “HCDR2”, “HCDR3”, “LCDR1”, “LCDR2” and “LCDR3” as used herein includes CDRs defined by any of the methods described supra, e.g., by Kabat, Chothia & Lesk, or IMGT. Two antibodies are determined to have the same CDR as one another with respect to a HCDR1, HCDR2, HCDR3, LCDR1, LCDR2 and/or LCDR3, when the identity of that CDR is determined for both antibodies using the same method.

In some embodiments, the anti-CD38 antibody comprises:
a) a heavy chain complementarity determining region 1 (HCDR1), HCDR2 and HCDR3 amino acid sequences of SEQ ID NOs:6, 7 and 8, respectively;
b) a light chain complementarity determining region 1 (LCDR1), LCDR2 and LCDR3 amino acid sequences of SEQ ID NOs:9, 10 and 11, respectively; or
c) both a) and b).

In some embodiments, the anti-CD38 antibody comprises:
a) a HCDR1, HCDR2 and HCDR3 amino acid sequences of SEQ ID NOs:6, 7 and 8, respectively; and
b) a LCDR1, LCDR2 and LCDR3 amino acid sequences of SEQ ID NOs:9, 10 and 11, respectively.

In some embodiments, the anti-CD38 antibody comprises a heavy chain variable region (VH) amino acid sequence of SEQ ID NO:4. In some embodiments, the anti-CD38 antibody comprises a VH amino acid sequence that is at least 90% identical, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:4. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%.

As used herein, the term “identical” or “has sequence identity,” refers to the extent to which two amino acid sequences have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same amino acid upon alignment of the reference and test sequences to achieve a maximal level of identity. As an example, two sequences are considered to have 70% sequence identity when, upon alignment to achieve a maximal level of identity, the test sequence has the same amino acid residue at 70% of the same positions over the entire length of the reference sequence.

In some embodiments, the anti-CD38 antibody comprises a light chain variable region (VL) amino acid sequence of SEQ ID NO:5. In some embodiments, the anti-CD38 antibody comprises a VL amino acid sequence that is at least 90% identical, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:5. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%.

In some embodiments, the anti-CD38 antibody comprises:
a) a VH amino acid sequence that is at least 95% identical to SEQ ID NO:4;
b) a VL amino acid sequence that is at least 95% identical to SEQ ID NO:5; or
c) both a) and b).

In some embodiments, the anti-CD38 antibody comprises:
a) a VH amino acid sequence that is at least 95% identical to SEQ ID NO:4; and
b) a VL amino acid sequence that is at least 95% identical to SEQ ID NO:5.

In certain embodiments, the anti-CD38 antibody comprises:
a) a VH amino acid sequence of SEQ ID NO:4;
b) a VL amino acid sequence of SEQ ID NO:5; or
c) both a) and b).

In particular embodiments, the anti-CD38 antibody comprises:
a) a VH amino acid sequence of SEQ ID NO:4; and
b) a VL amino acid sequence of SEQ ID NO:5.

In some embodiments, the anti-CD38 antibody comprises a heavy chain amino acid sequence that is at least 80% identical, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:12. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the anti-CD38 antibody comprises a heavy chain amino acid sequence of SEQ ID NO:12.

In some embodiments, the anti-CD38 antibody comprises a light chain amino acid sequence that is at least 80% identical, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:13. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the anti-CD38 antibody comprises a light chain amino acid sequence of SEQ ID NO:13.

In some embodiments, the anti-CD38 antibody comprises:
a) a heavy chain amino acid sequence that is at least 95% identical to SEQ ID NO:12;
b) a light chain amino acid sequence that is at least 95% identical to SEQ ID NO:13; or
c) both a) and b).

In some embodiments, the anti-CD38 antibody comprises:
a) a heavy chain amino acid sequence that is at least 95% identical to SEQ ID NO:12; and
b) a light chain amino acid sequence that is at least 95% identical to SEQ ID NO:13.

In some embodiments, the anti-CD38 antibody comprises:
a) a heavy chain amino acid sequence of SEQ ID NO:12;
b) a light chain amino acid sequence of SEQ ID NO:13; or
c) both a) and b).

In some embodiments, the anti-CD38 antibody comprises:
a) a heavy chain amino acid sequence of SEQ ID NO:12; and
b) a light chain amino acid sequence of SEQ ID NO:13.

In some embodiments, the anti-CD38 antibody comprises the HCDR1, HCDR2, HCDR3, LCDR1, LCDR2, and LCDR3 amino acid sequences of:
a) the VH of SEQ ID NO:14 and the VL of SEQ ID NO:15;
b) the VH of SEQ ID NO:16 and the VL of SEQ ID NO:17;
c) the VH of SEQ ID NO:18 and the VL of SEQ ID NO:19; or
d) the VH of SEQ ID NO:20 and the VL of SEQ ID NO:21.

In some embodiments, the anti-CD38 antibody comprises the VH and VL amino acid sequences of:
a) SEQ ID NOs:14 and 15, respectively;
b) SEQ ID NOs:16 and 17, respectively;
c) SEQ ID NOs:18 and 19, respectively; or
d) SEQ ID NOs:20 and 21, respectively.

Immunoglobulins may be assigned to five major classes: IgA, IgD, IgE, IgG and IgM, depending on the heavy chain constant domain amino acid sequence. IgA is further sub-classified as the isotypes IgA1, IgA2. IgG is further sub-classified as IgG1, IgG2, IgG3 and IgG4. Antibody light chains of any vertebrate species can be assigned to one of two clearly distinct types, namely kappa (κ) and lambda (λ), based on the amino acid sequences of their constant domains.

In some embodiments, the anti-CD38 antibody is of IgG1, IgG2, IgG3 or IgG4 subtype. In some embodiments, the anti-CD38 antibody is of IgG1 subtype. Some variation exists within the IgG1 constant domain (e.g., well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The anti-CD38 antibody may be of any IgG1 allotype, such as G1m17, G1m3, G1m1, G1m2, G1m27 or G1m28. In some embodiments, the anti-CD38 antibody is of κ subtype. In some embodiments, the anti-CD38 antibody is of IgG1/κ subtype.

The antibody can be of any species, such as a murine antibody, a human antibody, a chimeric antibody (e.g., humanized antibody). In some embodiments, the anti-CD38 antibody is a human antibody.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region is also derived from sequences of human origin. Antibodies in which antigen binding sites are derived from a non-human species are not included in the definition of “human antibody.”

A human antibody comprises heavy or light chain variable regions that are derived from sequences of human origin if the variable regions of the antibody are obtained from a system that uses human germline immunoglobulin or rearranged immunoglobulin genes. Non-limiting example systems include human immunoglobulin gene libraries displayed on phage, and transgenic non-human animals such as mice or rats carrying human immunoglobulin loci. A human antibody typically contains amino acid differences when compared to the human germline or rearranged immunoglobulin sequences due to, for example, naturally occurring somatic mutations, intentional substitutions in the framework or antigen binding site, and substitutions introduced during cloning or VDJ recombination in non-human animals. Typically, a human antibody is at least 80% identical in amino acid sequence to an amino acid sequence encoded by a human germline or rearranged immunoglobulin gene. For example, about: 80%, 81%, 82%, 83%, 84%, 85%, 86%, 87%, 88%, 89%, 90%, 91%, 92%, 93%, 94%, 95%, 96%, 97%, 98%, 99% or 100% identical. In some cases, a human antibody may contain consensus framework sequences derived from human framework sequence analyses (see, e.g., Knappik et al., J. Mol. Biol. 296:57-86 (2000)), or synthetic HCDR3 incorporated into human immune-globulin gene libraries displayed on phage (see, e.g., Shi et al., J. Mol. Biol. 397:385-96 (2010) and Int. Pat. Publ. No. WO2009/085462).

In some embodiments, the anti-CD38 antibody is daratumumab. Daratumumab is of IgG1/κ subtype and is described in U.S. Pat. No. 7,829,673. Daratumumab comprises a HCDR1, HCDR2 and HCDR3 amino acid sequences of SEQ ID NOs:6, 7 and 8, respectively; and a LCDR1, LCDR2 and LCDR3 amino acid sequences of SEQ ID NOs:9, 10 and 11, respectively. Daratumumab comprises a VH amino acid sequence of SEQ ID NO:4, and a VL amino acid sequence of SEQ ID NO:5. Daratumumab comprises a heavy chain amino acid sequence of SEQ ID NO:12, and a light chain amino acid sequence of SEQ ID NO:13.

In some embodiments, daratumumab is DARZALEX® brand of daratumumab or a biosimilar of DARZALEX® brand of daratumumab. Daratumumab can be prepared by any method known in the art for preparing monoclonal antibodies including, but not limited to, hybridoma production. For example, daratumumab can be produced in a mammalian cell line (e.g., CHO cell line) using recombinant DNA technology. Daratumumab and methods of producing daratumumab are further described in, e.g., WO2006099875, U.S. Pat. No. 7,829,673, US2015246123, and de Weers et al. J. Immunol. 186: 1840-48 (2011), the contents of which are incorporated herein by reference.

In some embodiments, the anti-CD38 antibody comprises a mutation in at least one amino acid residue selected from those corresponding to E345, E430, S440, Q386, P247, 1253, S254, Q311, D/E356, T359, E382, Y436, and K447 in the Fc-region of a human IgG1 heavy chain, to increase an effector function. Non-limiting examples of the effector functions include antibody-dependent cell-mediated cytotoxicity (ADCC), antibody-dependent cellular phagocytosis (ADCP), binding to complement receptor of an opsonized antibody mediated by the antibody, C1q-binding, complement activation, complement-dependent cellular cytotoxicity (CDCC), complement-dependent cytotoxicity (CDC), complement-enhanced cytotoxicity, downmodulation, Fc-gamma receptor-binding, FcRn-binding, induction of apoptosis, internalization, oligomer (e.g., hexamer) formation, oligomer (e.g., hexamer) stability, opsonization, Protein A-binding and Protein G-binding. Non-limiting examples of mutations, e.g., ones that increases hexamer formation, hexamer stability or both can be found in Int. Pat. Publ. Nos. WO 13/004842 and WO 20/012036, incorporated by reference in their entirety. In some embodiments, the anti-CD38 antibody is HexaBody-CD38 (GEN3014).

Other non-limiting examples of anti-CD38 antibodies that may be used in the methods of the invention include mAb003, mAb024, MOR-202 (MOR-03087), Isatuximab, and anti-CD38 antibodies described in Int. Pat. Publ. Nos. WO05/103083, WO06/125640, WO07/042309, WO08/047242 and WO14/178820, etc. MAb003, comprising the VH and the VL amino acid sequences of SEQ ID NOs:14 and 15, respectively, is described in U.S. Pat. No. 7,829,673. MAb024, comprising the VH and the VL amino acid sequences of SEQ ID NOs:16 and 17, respectively, is described in U.S. Pat. No. 7,829,673. MOR-202 (MOR-03087), comprising the VH and the VL amino acid sequences of SEQ ID NOs:18 and 19, respectively, is described in U.S. Pat. No. 8,088,896. Isatuximab, comprising the VH and the VL amino acid sequences of SEQ ID NOs:20 and 21, respectively, is described in U.S. Pat. No. 8,153,765. The VH and the VL of mAb003, mAb024, MOR-202 or Isatuximab, or a combination thereof, may be expressed as IgG1/κ.

Anti-CD38 antibodies used in the methods of the invention may also be selected de novo from, e.g., a phage display library, where the phage is engineered to express human immunoglobulins or portions thereof such as Fabs, single chain antibodies (scFv), or unpaired or paired antibody variable regions (Knappik et al., J. Mol. Biol. 296:57-86 (2000); Krebs et al., J. Immunol. Meth. 254:67-84 (2001); Vaughan et al., Nature Biotechnology 14:309-14 (1996); Sheets et al., PITAS (USA) 95:6157-62 (1998); Hoogenboom & Winter, J. Mol. Biol. 227:381 (1991); Marks et al., J. Mol. Biol. 222:581 (1991)). CD38 binding variable domains may be isolated from e.g., phage display libraries expressing antibody heavy and light chain variable regions as fusion proteins with bacteriophage pIX coat protein as described in Shi et al., J. Mol. Biol. 397:385-96 (2010) and Intl. Pat. Publ. No. WO09/085462. The antibody libraries may be screened for binding to human CD38 extracellular domain; obtained positive clones further characterized; Fabs isolated from the clone lysates, and subsequently cloned as full-length antibodies. Such phage display methods for isolating human antibodies are established in the art. See for example: U.S. Pat. Nos. 5,223,409, 5,403,484, 5,427,908, 5,571,698, 5,580,717, 5,885,793, 5,969,108, 6,172,197, 6,521,404, 6,544,731, 6,555,313, 6,582,915 and 6,593,081.

In some embodiments, the anti-CD38 antibody binds human CD38 with a dissociation constant (KD) of less than about: 1×10⁻⁷, 1×10⁻⁸, 1×10⁻⁹, 1×10⁻¹⁰, 1×10⁻¹¹, 1×10⁻¹², 1×10⁻¹³, 1×10⁻¹⁴ or 1×10⁻¹⁵ M, as determined by surface plasmon resonance or the KinExA method, as practiced by those of skill in the art. In some embodiments, the antibody binds human CD38 with a K_{D} of less than about 1×10⁻⁸ M. In some embodiments, the antibody binds human CD38 with a K_{D} of less than about 1×10⁻⁹ M.

KinExA instrumentation, ELISA or competitive binding assays are known to those skilled in the art. The measured affinity of a particular antibody/CD38 interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer. Those skilled in the art will appreciate that the internal error for affinity measurements, for example, using Biacore 3000 or ProteOn (measured as standard deviation, SD) may typically be within 5-33% for measurements within the typical limits of detection. Therefore, the term “about” in the context of K_{D} reflects the typical standard deviation in the assay. For example, the typical SD for a K_{D} of 1×10⁻⁹ M is up to ±0.33×10⁻⁹ M.

The term “antigen-binding fragment” refers to a portion of an immunoglobulin molecule (e.g., an antibody) that retains the antigen binding properties of the parental full-length antibody. Non-limiting examples of antigen-binding fragments include HCDR1, 2 and/or 3, LCDR1, 2 and/or 3, a V_{H} region, a V_{L} region, an Fab fragment, an F(ab′)₂ fragment, an Fd fragment, an Fv fragment, and a domain antibody (dAb) consisting of one V_{H} domain or one V_{L} domain. V_{H} and V_{L} domains may be linked together via a synthetic linker to form various types of single-chain antibody designs in which the V_{H}/V_{L} domains pair intramolecularly, or intermolecularly in those cases when the V_{H} and V_{L} domains are expressed by separate chains, to form a monovalent antigen binding site, such as single chain Fv (scFv) or diabody. See, for example, Int. Pat. Publ. Nos. WO1998/44001, WO1988/01649, WO1994/13804 and WO1992/01047.

In some embodiments, the anti-CD38 antibody is a human monoclonal antibody (mAb) or an antigen binding fragment thereof.

The term “anti-CD38 antibody” is meant in a broad sense and includes multiparatopic antibodies; monospecific and multispecific (e.g., bispecific) antibodies; monoclonal antibodies (including murine, human, humanized and chimeric antibodies); dimeric, tetrameric and multimeric antibodies; single chain antibodies; domain antibodies and any other modified or engineered configuration of the immunoglobulin molecule that comprises an antigen binding site of the required specificity.

“Monoclonal antibody” refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. A monoclonal antibody may be monovalent, bivalent or multivalent. A monoclonal antibody may be monospecific or multispecific (e.g., bispecific). Monospecific antibodies bind one antigenic epitope. A multispecific antibody, such as a bispecific antibody or a trispecific antibody is included in the term monoclonal antibody.

“Multispecific” refers to an antibody that specifically binds at least two distinct antigens or at least two distinct epitopes within the antigens, for example three, four or five distinct antigens or epitopes. “Bispecific” refers to an antibody that specifically binds two distinct antigens or two distinct epitopes within the same antigen.

“Isolated antibody” refers to an antibody or an antigen-binding fragment thereof that is substantially free of other antibodies having different antigenic specificities (e.g., an isolated anti-CD38 antibody is substantially free of antibodies that specifically bind antigens other than human CD38). In the case of a bispecific antibody, the bispecific antibody specifically binds two antigens of interest, and is substantially free of antibodies that specifically bind antigens other than the two antigens of interest. In some embodiments, the anti-CD38 antibody is at least 80% pure, e.g., about: 80%, 81%, 82%, 83%, 84%, 85%, 86%, 87%, 88%, 89%, 90%, 91%, 92%, 93%, 94%, 95%, 96%, 97%, 98%, 99% or 100% pure.

“Recombinant” includes antibodies and other proteins that are prepared, expressed, created or isolated by recombinant means.

“Variant” refers to a polypeptide or a polynucleotide that differs from a reference polypeptide or a reference polynucleotide by one or more modifications, for example, substitutions, insertions, deletions or a combination thereof.

### Pharmaceutical Compositions

### Anti-CD38 Antibody

In methods of the disclosure, the anti-CD38 antibody may be provided in a suitable pharmaceutical composition.

In some embodiments, the pharmaceutical composition comprises from about 1,200 mg to about 5,000 mg of the anti-CD38 antibody, for example, about: 1,200-4,000, 1,300-4,000, 1,300-3,500, 1,400-3,500, 1,400-3,000, 1,500-3,000, 1,500-2,500, 1,600-2,500, 1,600-2,000, 1,700-2,000, 1,700-1,900 or 1,800-1,900 mg of the anti-CD38 antibody. In certain embodiments, the pharmaceutical composition comprises about: 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 3,000, 3,500, 4,000, 4,500 or 5,000 mg of the anti-CD38 antibody. In some embodiments, the pharmaceutical composition comprises about 1,200, 1,500, 1,800 or 2,000 mg of the anti-CD38 antibody. In particular embodiments, the pharmaceutical composition comprises about 1,800 mg of the anti-CD38 antibody.

The concentration of the anti-CD38 antibody included in the pharmaceutical compositions can vary. In some embodiments, the pharmaceutical composition comprises from about 1 mg/mL to about 180 mg/mL of the anti-CD38 antibody, for example, about: 2-180, 2-175, 5-175, 5-170, 10-180, 10-170, 10-165, 20-165, 20-160, 20-140, 20-120, 40-160, 40-155, 40-120, 60-155, 60-150, 60-120, 80-150, 80-145, 80-120, 100-145, 100-140, 100-120, 110-140, 110-135, 115-135, 115-130, 120-130 mg/mL of the anti-CD38 antibody.

In some embodiments, the pharmaceutical composition comprises about: 1, 2, 5, 10, 15, 20, 30, 40, 50, 60, 70, 80, 90, 100, 110, 120, 130, 140, 150, 160, 170 or 180 mg/mL of the anti-CD38 antibody. In certain embodiments, the pharmaceutical composition comprises about 100 mg/mL of the anti-CD38 antibody. In some embodiments, the pharmaceutical composition comprises about 140 mg/mL of the anti-CD38 antibody. In particular embodiments, the pharmaceutical composition comprises about 120 mg/mL of the anti-CD38 antibody.

The anti-CD38 antibody may be lyophilized for storage and reconstituted in a suitable carrier prior to use. This technique has been shown to be effective with conventional protein preparations and well known lyophilization and reconstitution techniques can be employed.

### Hyaluronidase

In some embodiments, a pharmaceutical composition suitable for use in a method of the disclosure is formulated for subcutaneous administration. Non-limiting examples of formulations suitable for subcutaneous administration include solutions, suspensions, emulsions, and dry products that can be dissolved or suspended in a pharmaceutically acceptable carrier for injection.

For subcutaneous administration of larger volumes, as typically needed for antibody solutions and compositions, the extracellular matrix of the subcutaneous tissue presents a problem. The space outside adipocytes in the hypodermis is not a fluid, but rather a solid extracellular matrix of collagenous fibrils embedded within a glycosaminoglycan-rich viscoelastic gel that buffers convective forces. The extracellular matrix limits the volume of drug that can be injected at a single site, as well as the rate and amount that reach the vascular compartment. Hyaluronidase is an enzyme that degrades hyaluronic acid (EC 3.2.1.35) and lowers the viscosity of hyaluronan in the extracellular matrix, thereby increasing tissue permeability. Thus, co-formulation or co-administration of an antibody with recombinant human hyaluronidase, such as rHuPH20, has allowed for increased injection volumes and bioavailability from subcutaneous injection.

In some embodiments, the pharmaceutical composition further comprises a hyaluronidase. In particular embodiments, the hyaluronidase is rHuPH20 recombinant hyaluronidase. rHuPH20 is a recombinant hyaluronidase (HYLENEX® recombinant) and is described in Int. Pat. Publ. No. WO2004/078140. In some embodiments, the hyaluronidase is rHuPH20 having the amino acid sequence of SEQ ID NO: 22.

Enzymatic activity of hyaluronidase, including rHuPH20 can be defined by units per mL (U/mL) or by total enzyme activity in a particular formulation (U). The standard definition for one unit (U) of enzyme activity is the amount of enzyme that catalyzes the reaction of 1 nmol of substrate per minute.

In some embodiments, the pharmaceutical composition comprises from about 750 U to about 75,000 U of the hyaluronidase. In certain embodiments, the pharmaceutical composition comprises from about 7,500 U to about 45,000 U of the hyaluronidase. In particular embodiments, the pharmaceutical composition comprises from about 30,000 U to about 45,000 U of the hyaluronidase.

In some embodiments, the pharmaceutical composition comprises about: 7,500, 8,000, 8,500, 9,000, 10,000, 15,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 55,000, 60,000, 65,000, 70,000 or 75,000 U of the hyaluronidase. In particular embodiments, the pharmaceutical composition comprises about 30,000 U of the hyaluronidase.

In some embodiments, the pharmaceutical composition comprises from about 50 U/mL to about 5,000 U/mL of the hyaluronidase, for example, about: 50-2,000, 500-5,000, 500-4,000, 500-2,000, 800-4,000, 800-3,500, 1,000-5,000, 1,000-3,500, 1,000-3,000, 1,200-3,000, 1,200-2,500, 1,500-2,500, 1,500-2,200, 1,800-2,200, 1,800-2,000, 2,000-5,000 U/mL.

In some embodiments, the pharmaceutical composition comprises about: 500 U/mL, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900 or 5,000 U/mL of the hyaluronidase.

In some embodiments, the pharmaceutical composition comprises about 50 U/mL of the hyaluronidase. In certain embodiments, the pharmaceutical composition comprises about 500 U/mL of the hyaluronidase. In other embodiments, the pharmaceutical composition comprises about 5,000 U/mL of the hyaluronidase. In particular embodiments, the pharmaceutical composition comprises about 2,000 U/mL of the hyaluronidase.

In some embodiments, the pharmaceutical composition comprises about: 1,200, 1,400, 1,600, 1,800, 2,000, 2,200, 2,400, 2,600, 2,800, 3,000 or 5,000 mg of the anti-CD38 antibody and about 30,000 U of the hyaluronidase. In certain embodiments, the pharmaceutical composition comprises about: 1,200, 1,400, 1,600, 1,800, 2,000, 2,200, 2,400, 2,600, 2,800, 3,000, or 5,000 mg of the anti-CD38 antibody and about 45,000 U of the hyaluronidase. In particular embodiments, the pharmaceutical composition comprises about 1,800 mg of the anti-CD38 antibody and about 30,000 U of the hyaluronidase.

Additional information regarding daratumumab and hyaluronidase can be found, for example, in the prescribing information product insert for DARZALEX FASPRO® (www_janssenlabels_com/package-insert/product-monograph/prescribing-information/DARZALEX+Faspro-pi.pdf), which is incorporated herein by reference.

Pharmaceutical composition referring to a product that results from combining an anti-CD38 antibody and a hyaluronidase includes both fixed and non-fixed combinations.

“Fixed combination” refers to a single pharmaceutical composition comprising two or more compounds, for example, the anti-CD38 antibody and the hyaluronidase administered simultaneously in the form of a single entity or dosage. In some embodiments, pharmaceutical composition comprising the anti-CD38 antibody and the hyaluronidase is a fixed combination.

“Non-fixed combination” refers to separate pharmaceutical compositions, wherein each comprises one or more compounds, for example, the anti-CD38 antibody and the hyaluronidase or unit dosage forms administered as separate entities either simultaneously, concurrently or sequentially with no specific intervening time limits, wherein such administration provides effective levels of the two compounds in the body of the subject. In some embodiments, pharmaceutical composition comprising the anti-CD38 antibody and the hyaluronidase is a non-fixed combination.

### Pharmaceutically Acceptable Carrier

In some embodiments, the pharmaceutical composition is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. The carrier may be diluent, adjuvant, excipient, or vehicle with which the anti-CD38 antibody is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the anti-CD38 antibody in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, Pa. 2006, Part 5, Pharmaceutical Manufacturing: 691-1092 (e.g., pages 958-89).

In some embodiments, a pharmaceutical composition suitable for use in methods of the disclosure further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing, and particularly antibody drug manufacturing. The carrier may be diluent, adjuvant, excipient, or vehicle with which the anti-CD38 antibody is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the anti-CD38 antibody in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, the pharmaceutical composition comprises histidine at a concentration of from about 1 mM to about 50 mM, e.g., about: 2-50, 2-40, 5-50, 5-40, 5-30, 5-20, 5-15, 5-10, 10-30 or 10-20 mM. In some embodiments, the pharmaceutical composition comprises histidine at a concentration of about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mM. In certain embodiments, the pharmaceutical composition comprises histidine at a concentration of about 10 mM. In particular embodiments, the pharmaceutical composition comprises L-histidine at a concentration of about 2.1 mM and L-histidine hydrochloride monohydrate at a concentration of about 5.9 (e.g., 5.8-5.9) mM.

In some embodiments, the pharmaceutical composition comprises methionine at a concentration of from about 0.1 mg/mL to about 5 mg/mL, e.g., about: 0.1-4.5, 0.1-4.0, 0.1-2.5, 0.2-5.0, 0.2-4.0, 0.2-3.5, 0.3-3.5, 0.3-3.0, 0.4-3.0, 0.4-2.5, 0.5-2.5, 0.5-4.0, 0.5-3.0, 0.5-2.0, 0.6-2.0, 0.6-1.5, 0.7-1.5, 0.7-1.2, 0.8-1.2, 0.8-1.0, 1.0-3.0 or 1.0-2.0 mg/mL. In certain embodiments, the pharmaceutical composition comprises methionine at a concentration of about: 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 mg/mL.

In some embodiments, the pharmaceutical composition comprises polysorbate.

In some embodiments, the pharmaceutical composition comprises polysorbate-20 (PS-20). In certain embodiments, the pharmaceutical composition comprises PS-20 at a concentration of from about 0.01% (w/v) to about 0.1% (w/v), e.g., about: 0.01-0.08%, 0.01-0.06%, 0.01-0.05%, 0.01-0.04%, 0.02-0.1%, 0.02-0.08%, 0.02-0.06%, 0.03-0.06%, 0.03-0.05%, 0.04-0.08% or 0.04-0.05% (w/v). In particular embodiments, the pharmaceutical composition comprises PS-20 at a concentration of from about 0.04% (w/v).

In some embodiments, the pharmaceutical composition comprises polysorbate-80 (PS-80). In certain embodiments, the pharmaceutical composition comprises PS-80 at a concentration of from about 0.01% (w/v) to about 0.08% (w/v), e.g., about: 0.01-0.04%, 0.02-0.1%, 0.02-0.08% or 0.04-0.08% (w/v). In some embodiments, the pharmaceutical composition comprises PS-80 at a concentration of about: 0.01% (w/v), 0.02% (w/v), 0.03% (w/v), 0.04% (w/v), 0.05% (w/v), 0.06% (w/v), 0.07% (w/v), 0.08% (w/v), 0.09% (w/v) or 0.1% (w/v).

In some embodiments, the pharmaceutical composition comprises PS-20 and PS-80. In certain embodiments, the pharmaceutical composition comprises PS-20 and PS-80 at a concentration of from about 0.01% (w/v) to about 0.08% (w/v), e.g., about: 0.01-0.04%, 0.02-0.1%, 0.02-0.08% or 0.04-0.08% (w/v). In some embodiments, the pharmaceutical composition comprises PS-20 and PS-80 at a concentration of about: 0.01% (w/v), 0.02% (w/v), 0.03% (w/v), 0.04% (w/v), 0.05% (w/v), 0.06% (w/v), 0.07% (w/v), 0.08% (w/v), 0.09% (w/v) or 0.1% (w/v).

In some embodiments, the pharmaceutical composition comprises saccharide.

In some embodiments, the pharmaceutical composition comprises saccharide at a concentration of from about 50 mM to about 500 mM, e.g., about: 50-450, 50-400, 50-350, 60-500, 60-450, 70-450, 70-400, 80-400, 80-350, 90-350, 90-300, 100-450, 100-400, 100-350, 100-300, 150-400, 150-350, 200-350, 200-325, 225-325, 225-300, 250-300 or 250-275 mM. In certain embodiments, the pharmaceutical composition comprises saccharide at a concentration of about: 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 mM.

In some embodiments, saccharide is sorbitol.

In some embodiments, the pharmaceutical composition comprises sorbitol at a concentration of from about 50 mM to about 500 mM, e.g., about: 50-450, 50-400, 50-350, 100-450, 100-400, 100-350, 100-300, 150-400, 150-350, 200-350, 200-325, 225-325, 225-300, 250-300 or 250-275 mM. In certain embodiments, the pharmaceutical composition comprises sorbitol ata concentration of about: 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 mM. In particular embodiments, the pharmaceutical composition comprises sorbitol at a concentration of about 270 mM.

In some embodiments, saccharide is sucrose.

In certain embodiments, the pharmaceutical composition comprises sucrose at a concentration of from about 50 mM to about 500 mM, e.g., about: 50-450, 50-400, 50-350, 100-350 or 100-200 mM. In some embodiments, the pharmaceutical composition comprises sucrose ata concentration of about: 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 mM.

In some embodiments, the pharmaceutical composition comprises mannitol.

In certain embodiments, the pharmaceutical composition comprises mannitol at a concentration of from about 100 mM to about 180 mM, e.g., about: 105-180, 105-175, 110-175, 110-170, 115-170, 115-165, 120-165, 120-160, 125-160, 125-155, 130-155, 130-150 or 140-180 mM. In some embodiments, the pharmaceutical composition comprises mannitol at a concentration of about: 100, 105, 110, 115, 120, 125, 130, 135, 140, 145, 150, 155, 160, 165, 170, 175 or 180 mM. In some embodiments, the pharmaceutical composition comprises mannitol at a concentration of about 140 mM.

In some embodiments, the pharmaceutical composition comprises acetic acid.

In certain embodiments, the pharmaceutical composition comprises acetic acid at a concentration of from about 1 mM to about 50 mM, e.g., about: 2-50, 2-45, 5-45, 5-40, 10-40, 10-35, 15-35, 15-30 or 20-30 mM. In some embodiments, the pharmaceutical composition comprises acetic acid at a concentration of about: 10, 15, 20, 25, 30, 35, 40, 45 or 50 mM. In particular embodiments, the pharmaceutical composition comprises acetic acid at a concentration of about 25 mM.

In some embodiments, the pharmaceutical composition comprises NaCl.

In some embodiments, the pharmaceutical composition comprises NaCl at a concentration of from about 20 mM to about 100 mM, e.g., about: 20-90, 30-90, 30-80, 40-80, 40-70 or 50-70 mM. In certain embodiments, the pharmaceutical composition comprises NaCl at a concentration of about: 20, 25, 30, 35, 40, 45, 50, 55, 60, 65, 70, 75, 80, 85, 90, 95 or 100 mM. In particular embodiments, the pharmaceutical composition comprises NaCl at a concentration of about 60 mM.

In some embodiments, the pharmaceutical composition comprises sodium acetate.

In some embodiments, the pharmaceutical composition comprises sodium acetate at a concentration of from about 10 mM to about 50 mM, e.g., about: 15-50, 15-45, 20-45, 20-40, 25-40 or 25-35 mM. In certain embodiments, the pharmaceutical composition comprises sodium acetate at a concentration of about: 10, 15, 20, 25, 30, 35, 40, 45 or 50 mM. In particular embodiments, the pharmaceutical composition comprises sodium acetate at a concentration of about 30 mM.

In some embodiments, the pharmaceutical composition is at from about pH 5.0 to about pH 7.0, e.g., from about pH 5.0 to about pH 6.0, from about pH 5.3 to about pH 5.8. In certain embodiments, the pharmaceutical composition is at about pH 5.5. In other embodiments, the pharmaceutical composition is at about pH 5.6.

In some embodiments, the pharmaceutically acceptable carrier comprises histidine, methionine, mannitol, sorbitol, polysorbate 20, polysorbate 80, or a combination thereof, and one or more salts (e.g., sodium chloride (NaCl), sodium acetate, etc.), wherein the pharmaceutical composition has a pH of 5 to 7.

In some embodiments, the pharmaceutical composition comprises:

about 1,800 mg of daratumumab,

about 30,000 units of rHuPH20 recombinant hyaluronidase,

about 4.9 mg L-histidine,

about 18.4 mg L-histidine hydrochloride monohydrate,

about 13.5 mg L-methionine,

about 6 mg polysorbate 20, and

about 735.1 mg sorbitol,

wherein the pharmaceutical composition has a pH of about 5.5.

In some embodiments, the pharmaceutical composition comprises:

about 120 mg/ml of daratumumab,

about 2,000 U/mL rHuPH20 recombinant hyaluronidase,

about 2.1 mM L-histidine,

about 5.8 mM L-histidine hydrochloride monohydrate,

about 6.0 mM L-methionine,

about 0.04% w/v polysorbate 20, and

about 270 mM sorbitol,

wherein the pharmaceutical composition has a pH of about 5.5.

In some embodiments, the pharmaceutical composition comprises:

about 1,800 mg of daratumumab,

about 30,000 units of rHuPH20 recombinant hyaluronidase,

about 4.9 mg L-histidine,

about 18.4 mg L-histidine hydrochloride monohydrate,

about 13.5 mg L-methionine,

about 6 mg polysorbate 20, and

about 735.1 mg sorbitol,

wherein the pharmaceutical composition has a pH of about 5.6.

In some embodiments, the pharmaceutical composition comprises:

about 120 mg/ml of daratumumab,

about 2,000 U/mL rHuPH20 recombinant hyaluronidase,

about 2.1 mM L-histidine,

about 5.8 mM L-histidine hydrochloride monohydrate,

about 6.0 mM L-methionine,

about 0.04% w/v polysorbate 20, and

about 270 mM sorbitol,

wherein the pharmaceutical composition has a pH of about 5.6.

In some embodiments, the pharmaceutical composition comprises:
a) from about 60 mg/mL to about 180 mg/mL (e.g., about 120 mg/mL) of the anti-CD38 antibody in about 25 mM acetic acid, about 60 mM sodium chloride, about 140 mM mannitol and about 0.04% w/v PS-20, at pH about 5.5; and
b) about from 30,000 U to about 45,000 U of the hyaluronidase in 10 mM L-histidine, 130 mM NaCl, 10 mM L-methionine, 0.02% w/v PS-80, at pH about 6.5.

In some embodiments, the pharmaceutical composition comprises:
a) from about 60 mg/mL to about 180 mg/mL (e.g., about 120 mg/mL) of the anti-CD38 antibody in about 25 mM acetic acid, about 60 mM sodium chloride, about 140 mM mannitol and about 0.04% w/v PS-20, at pH about 5.5; and
b) about 30,000 U of the hyaluronidase in 10 mM L-histidine, 130 mM NaCl, 10 mM L-methionine, 0.02% w/v PS-80, at pH about 6.5.

In some embodiments, the pharmaceutical composition comprises:
a) from about 60 mg/mL to about 180 mg/mL (e.g., about 120 mg/mL) of the anti-CD38 antibody in about 25 mM acetic acid, about 60 mM sodium chloride, about 140 mM mannitol and about 0.04% w/v PS-20, at pH about 5.5; and
b) about 45,000 U of the hyaluronidase in 10 mM L-histidine, 130 mM NaCl, 10 mM L-methionine, 0.02% w/v PS-80, at pH about 6.5.

In some embodiments, the pharmaceutical composition comprises:
a) from about 1 mg/mL to about 180 mg/mL of the anti-CD38 antibody;
b) from about 50 U/ml to about 5,000 U/ml of the hyaluronidase;
c) from about 5 mM to about 50 mM histidine; and
d) from about 50 mM to about 400 mM sorbitol,
optionally, the pharmaceutical composition further comprises:
e) from about 0.01% w/v to about 0.1% w/v PS-20; and/or
f) from about 0.1 mg/mL to about 2.5 mg/mL methionine.

In some embodiments, the pharmaceutical composition comprises:
a) from about 100 mg/mL to about 140 mg/mL (e.g., about 120 mg/mL) of the anti-CD38 antibody;
b) from about 50 U/ml to about 5,000 U/ml of the hyaluronidase;
c) about 10 mM histidine; and
d) from about 100 mM to about 300 mM sorbitol,
optionally, the pharmaceutical composition further comprises:
e) from about 0.01% w/v to about 0.04% w/v PS-20; and
f) from about 1 mg/mL to about 2 mg/mL methionine.

In some embodiments, the pharmaceutical composition comprises:
a) about 120 mg/mL of the anti-CD38 antibody;
b) about 2,000 U/ml of rHuPH20;
c) about 10 mM histidine;
d) about 300 mM sorbitol;
e) about 0.04% w/v PS-20; and
f) about 1 mg/mL methionine; pH about 5.5.

In some embodiments, the pharmaceutical composition comprises:
a) about 120 mg/mL of the anti-CD38 antibody;
b) about 2,000 U/ml of rHuPH20;
c) about 10 mM histidine;
d) about 300 mM sorbitol;
e) about 0.04% w/v PS-20; and
f) about 2 mg/mL methionine; pH about 5.5.

In some embodiments, the pharmaceutical composition comprises:
a) about 120 mg/mL of the anti-CD38 antibody;
b) about 2,000 U/ml of rHuPH20;
c) about 10 mM histidine;
d) about 300 mM sorbitol;
e) about 0.01% w/v PS-20; and
f) about 2 mg/mL methionine; pH about 5.5.

In some embodiments, the pharmaceutical composition comprises:
a) about 120 mg/mL of the anti-CD38 antibody;
b) about 2,000 U/ml of rHuPH20;
c) about 10 mM histidine;
d) about 300 mM sorbitol;
e) about 0.02% w/v PS-20; and
f) about 2 mg/mL methionine; pH about 5.5.

In some embodiments, the pharmaceutical composition comprises:
a) about 120 mg/mL of the anti-CD38 antibody;
b) about 2,000 U/ml of rHuPH20;
c) about 10 mM histidine;
d) about 300 mM sorbitol;
e) about 0.06% w/v PS-20; and
f) about 2 mg/mL methionine; pH about 5.5.

Pharmaceutical compositions comprising the anti-CD38 antibody can be prepared by any method known in the art in view of the present disclosure. For example, the anti-CD38 antibody can be mixed with one or more pharmaceutically acceptable excipients to obtain a solution. The solution can be stored as a liquid at a temperature of about 2° C. to 8° C. and under protection from light exposure in an appropriate vial until administered to the subject.

In some embodiments, the pharmaceutical composition is prepared by mixing about 20 mg/ml of the anti-CD38 antibody with about 1.0 mg/mL rHuPH20 (75-150 kU/mL) prior to administration of the mixture to the subject, wherein the anti-CD38 antibody is in about 25 mM sodium acetate, about 60 mM sodium chloride, about 140 mM D-mannitol, about 0.04% polysorbate 20, at about pH 5.5, and rHuPH20 is in about 10 mM L-histidine, about 130 mM NaCl, about 10 mM L-methionine, and about 0.02% polysorbate 80, at about pH 6.5.

### Administration

“Treat,” “treating” or “treatment” refers to therapeutic treatment wherein the object is to slow down (lessen) an undesired physiological change or disease, such as the development or spread of tumor or tumor cells, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, lack of metastasis, amelioration or palliation of the disease state, and remission (whether partial or total), whether detectable or undetectable. “Treatment” may also mean prolonging survival as compared to expected survival if a subject was not receiving treatment. Those in need of treatment include those subjects already with the undesired physiological change or disease well as those subjects prone to have the physiological change or disease.

Daratumumab is indicated for the treatment of adult patients with multiple myeloma. For example, as monotherapy, in patients who have received at least three prior lines of therapy including a proteasome inhibitor (PI) and an immunomodulatory agent or who are double-refractory to a PI and an immunomodulatory agent. Additional information regarding daratumumab can be found, for example, in the prescribing information product insert for DARZALEX® (www_janssenlabels_com/package-insert/product-monograph/prescribing-information/DARZALEX-pi.pdf), which is incorporated herein by reference.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics include, for example, improved well-being of the patient, reduction in a tumor burden, arrested or slowed growth of a tumor, and/or absence of metastasis of cancer cells to other locations in the body.

“Inhibits growth” (e.g., referring to tumor cells) refers to a measurable decrease in the tumor cell growth or tumor tissue in vitro or in vivo when contacted with a therapeutic or a combination of therapeutics or drugs, when compared to the growth of the same tumor cells or tumor tissue in the absence of the therapeutic or the combination of therapeutic drugs. Inhibition of growth of a tumor cell or tumor tissue in vitro or in vivo may be at least about 10%, 20%, 30%, 40%, 50%, 60%, 70%, 80%, 90%, 99%, or 100%.

The anti-CD38 antibody may also be administered prophylactically to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The mode of administration of the anti-CD38 antibody may be any suitable parenteral administration. Non-limiting examples of administration include intradermal, intramuscular, intraperitoneal, intravenous, subcutaneous, pulmonary, transmucosal (oral, intranasal, intravaginal, rectal), etc.

In some embodiments, the anti-CD38 antibody is administered subcutaneously. “Subcutaneous administration” refers to administration under the skin, in which a drug or therapeutic is injected into the tissue layer between the skin and muscle. Medication administered via subcutaneous administration is usually absorbed more slowly than if injected into a vein.

“Dosage” refers to the information of the amount of the therapeutic or the drug to be taken by the subject and the frequency of the number of times the therapeutic is to be taken by the subject. “Dose” refers to the amount or quantity of the therapeutic or the drug to be taken each time.

In some embodiments, a dose of the anti-CD38 antibody is from about 10 mg to about 2,400 mg per administration, for example, about: 10-2,400, 10-2,000, 20-2,000, 20-1,500, 50-1,500, 50-1,000, 100-1,000, 100-500 or 200-500 mg per administration. In certain embodiments, a dose of the anti-CD38 antibody is about: 700, 800, 900, 1,000, 1,100, 1,200, 1,300, 1,400, 1,500, 1,600, 1,700, 1,800, 1,900, 2,000, 2,100, 2,200, 2,300 or 2,400 mg, per administration. In particular embodiments, a dose of the anti-CD38 antibody is about 1,800 mg per administration.

In some embodiments, the anti-CD38 antibody is subcutaneously administered without recombinant human hyaluronidase. In other embodiments, the anti-CD38 antibody is subcutaneously administered with recombinant human hyaluronidase.

The pharmaceutical compositions to be administered may comprise about 1,800 mg of the anti-CD38 antibody and about 30,000 U of hyaluronidase. In some embodiments, the concentration of the anti-CD38 antibody in the pharmaceutical composition is about 120 mg/ml. The pharmaceutical composition comprising the anti-CD38 antibody and the hyaluronidase may be administered subcutaneously to the abdominal region.

The pharmaceutical compositions of the invention may be administered as a non-fixed combination. The pharmaceutical compositions of the invention may also be administered as a fixed combination, e.g., as a unit dosage form (or dosage unit form). Fixed combinations may be advantageous for ease of administration and uniformity of dosage.

In some embodiments, the pharmaceutical composition (e.g., comprising the anti-CD38 antibody and the hyaluronidase) is administered in a total volume of about: 10, 11, 12, 13, 14, 15, 16, 17, 18, 19, 20, 25, 30, 35, 40, 45, 50, 55, 60, 65, 70, 75, 80, 85, 90, 95, 100, 105, 110, 115 or 120 mL. In certain embodiments, the pharmaceutical composition is administered in a total volume of about: 80, 90, 100, 110 or 120 mL. In other embodiments, the pharmaceutical composition is administered in a total volume of about 10-20 mL, e.g., about: 10, 11, 12, 13, 14, 15, 16, 17, 18, 19 or 20 mL. In particular embodiments, the pharmaceutical composition has a total volume of about 15 mL. The total volume of administration is typically smaller for the fixed combinations when compared to the non-fixed combinations.

The pharmaceutical compositions of the disclosure may also be administered as a fixed combination, e.g., as a unit dosage form.

In some embodiments, the unit dosage form comprises:
the anti-CD38 antibody in an amount of about 1,200 mg to about 4,000 mg, and
optionally, rHuPH20 in an amount of about 30,000 U to about 75,000 U.

In some embodiments, the unit dosage form comprises:
the anti-CD38 antibody in an amount of about 1,200 mg to about 2,400 mg, and
optionally, rHuPH20 in an amount of about 30,000 U to about 45,000 U.

In some embodiments, the unit dosage form comprises:
the anti-CD38 antibody in an amount of about 1,200 mg to about 1,800 mg, and
optionally, rHuPH20 in an amount of about 30,000 U to about 45,000 U.

In some embodiments, the unit dosage form comprises:
the anti-CD38 antibody in an amount of about 1,200 mg to about 5,000 mg (e.g., about 1,800 mg),
histidine at a concentration of about 5 mM to about 15 mM (e.g., about 7.9 mM),
sorbitol at a concentration of about 100 mM to about 300 mM (e.g., about 270 mM),
PS-20 at a concentration of about 0.01% (w/v) to about 0.05% (w/v) (e.g., about 0.04% (w/v)),
methionine at a concentration of about 0.8 mg/mL to about 2 mg/mL (e.g., about 0.9 mg/mL), and
optionally, rHuPH20 in an amount of about 30,000 U to about 75,000 U (e.g., about 30,000 U),
wherein the pharmaceutical composition has a pH of about 5.5 or about 5.6.

In some embodiments, the unit dosage form comprises:
the anti-CD38 antibody in an amount of about 1,200 mg to about 2,400 mg (e.g., about 1,800 mg),
histidine at a concentration of about 5 mM to about 10 mM (e.g., about 7.9 mM),
sorbitol at a concentration of about 250 mM to about 300 mM (e.g., about 270 mM),
PS-20 at a concentration of about 0.03-0.05% (w/v) (e.g., about 0.04% (w/v)),
methionine at a concentration of from about 0.8-1 mg/mL (e.g., about 0.9 mg/mL), and
optionally, rHuPH20 in an amount of about 30,000 U to about 45,000 U (e.g., about 30,000 U),
wherein the pharmaceutical composition has a pH of about 5.5 or about 5.6.

In some embodiments, the unit dosage form comprises:
the anti-CD38 antibody in an amount of about 1,800 mg,
histidine at a concentration of about 7.9 mM,
sorbitol at a concentration of about 270 mM,
PS-20 at a concentration of about 0.04% (w/v)
methionine at a concentration of about 0.9 mg/mL, and
optionally, rHuPH20 in an amount of about 30,000 U,
wherein the pharmaceutical composition has a pH of about 5.5 or about 5.6.

In some embodiments, the unit dosage form comprises:
the anti-CD38 antibody in an amount of about 1,800 mg,
histidine at a concentration of about 7.9 mM,
sorbitol at a concentration of about 270 mM,
PS-20 at a concentration of about 0.04% (w/v),
methionine at a concentration of about 0.9 mg/mL, and
optionally, rHuPH20 in an amount of about 45,000 U,
wherein the pharmaceutical composition has a pH of about 5.5 or about 5.6.

In some embodiments, the unit dosage form of the pharmaceutical composition is stored in a container selected from a vial, a cartridge, a syringe, a prefilled syringe or a disposable pen.

In some embodiments, the total dosage of the anti-CD38 antibody can be administered to the subject in a single subcutaneous injection, or in multiple subcutaneous injections, such as 1, 2, 3, 4, 5, or more subcutaneous injections.

In some embodiments, each subcutaneous injection lasts about 1 minute to about 60 minutes, e.g., about: 10-55, 15-55, 15-50, 20-50, 20-45, 25-45, 25-40, 30-40, 1-10, 1-9, 1-8, 1-7, 1-6, 1-5, 2-10, 2-9, 2-8, 2-7, 2-6, 2-5, 3-10, 3-9, 3-8, 3-7, 3-6 or 3-5 minutes. In certain embodiments, each subcutaneous injection lasts about: 1, 2, 3, 4, 5, 6, 7, 8, 9, 10, 15, 20, 25, 30, 35, 40, 45, 50, 55 or 60 minutes. In particular embodiments, each subcutaneous injection lasts about 3 minutes to about 5 minutes.

In some embodiments, the pharmaceutical composition is administered once per day, once per week, once every 2 weeks, once per month, once every 2 months, once every 3 months, once every 4 months, once every 6 months, once every 9 months, for a period of one day, one week, 2 weeks, 3 weeks, one month, 2 months, 3 months, 4 months, 5 months, 6 months, 9 months, 1 year, 18 months, or 2 years or longer.

The administration of the pharmaceutical composition (e.g., comprising the anti-CD38 antibody and the hyaluronidase) may be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical composition comprising the anti-CD38 antibody and the hyaluronidase may be administered once weekly for eight weeks, followed by once in two weeks for 16 weeks, followed by once in four weeks.

In some embodiments, the anti-CD38 antibody is administered once weekly, every 2 weeks, or every 4 weeks during a 28-day cycle. In some embodiments, the anti-CD38 antibody is administered once weekly during Cycle 1, every 2 weeks during Cycles 2-5, and every 4 weeks thereafter.

In some embodiments, the total dosage of the pharmaceutical composition is administered to the subject in a single subcutaneous injection per administration. In particular embodiments, the pharmaceutical composition is administered in a total volume of about 15 mL.

In some embodiments, the total dosage of the pharmaceutical composition is administered to the subject in multiple subcutaneous injections per administration, such as 2, 3, 4, or 5 subcutaneous injections.

In some embodiments, the anti-CD38 antibody is administered intravenously (e.g., by intravenous infusion).

In some embodiments, a total dosage of the anti-CD38 antibody is from about 10 mg to about 600 mg per administration, for example, about: 10-550, 15-550, 15-500, 25-500, 25-450, 40-450, 40-400, 60-400, 60-350, 100-350, 100-300, 150-300, 150-250 or 200-250 mg per administration. In certain embodiments, a total dosage of the anti-CD38 antibody is about: 10, 20, 25, 30, 40, 50, 60, 70, 75, 100, 125, 150, 175, 200, 225, 250, 275, 300, 325, 350, 375, 400, 425, 450, 475, 500, 525, 550, 575 or 600 mg, per administration.

The administration of the anti-CD38 antibody may be repeated. For example, after 1, 2, 3, 4, 5 or 6 days, 1, 2, 3, 4, 5, 6 or 7 weeks, or 1, 2, 3, 4, 5 or 6 months, or longer. Repeated courses of treatment are also possible, as is chronic administration. The repeated administration may be at the same dose or at a different dose. For example, the anti-CD38 antibody may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

In some embodiments, the anti-CD38 antibody is administered at 16 mg/kg once a week for 8 weeks, followed by administration at 16 mg/kg once every two weeks for 16 weeks, followed by administration at 16 mg/kg once every four weeks until discontinuation.

In some embodiments, the anti-CD38 antibody is administered at 8 mg/kg once a week for 8 weeks, followed by administration at 8 mg/kg once every two weeks for 16 weeks, followed by administration at 8 mg/kg once every four weeks until discontinuation.

In some embodiments, the anti-CD38 antibody is administered at 16 mg/kg once a week for 4 weeks, followed by administration at 16 mg/kg once every two weeks for 16 weeks, followed by administration at 16 mg/kg once every four weeks until discontinuation.

In some embodiments, the anti-CD38 antibody is administered at 8 mg/kg once a week for 4 weeks, followed by administration at 8 mg/kg once every two weeks for 16 weeks, followed by administration at 8 mg/kg once every four weeks until discontinuation.

In some embodiments, the intravenous infusion is given over 15, 30, 45 or 60 minutes. In some embodiments, the intravenous infusion is given over 1.5, 2, 3, 4, 5, 6, 7, 8, 9, 10, 11 or 12 hours.

The dose of the anti-CD38 antibody given to a patient is sufficient to alleviate or at least partially arrest the disease being treated (“therapeutically effective amount”). Non-limiting examples of therapeutically effective amounts include about 0.005 mg to about 100 mg/kg, e.g. about: 0.05-30, 5-25, 1, 2, 3, 4, 5, 6, 7, 8, 9, 10, 15, 16, 17, 18, 19, 20, 21, 22, 23, 24, 25, 30, 40, 50, 60, 70, 80, 90 or 100 mg/kg.

For example, the anti-CD38 antibody may be provided as a daily dosage in an amount of about 0.1-100 mg/kg, such as about 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

A fixed unit dose may also be given, for example, 50, 100, 200, 500 or 1000 mg. In some embodiments, the dose is based on the patient's surface area, e.g., 500, 400, 300, 250, 200, or 100 mg/m². The dosage may also depend on the disease. Usually between 1 and 8 doses, e.g., 1, 2, 3, 4, 5, 6, 7 or 8, may be administered to treat MM. In some embodiments, 9, 10, 11, 12, 13, 14, 15, 16, 17, 18, 19, 20 or more doses may be administered.

The anti-CD38 antibody may be administered as maintenance therapy, such as, e.g., once a week for a period of 6 months or more.

In certain embodiments, a pharmaceutical composition comprising about 1,800 mg of the anti-CD38 antibody (e.g., daratumumab) and about 30,000 U of the hyaluronidase (e.g., rHuPH20 recombinant hyaluronidase) is administered on 28-day cycles, once weekly during Cycle 1, every 2 weeks during Cycles 2-5, and every 4 weeks thereafter.

In particular embodiments, a pharmaceutical composition comprising about 1,800 mg of the anti-CD38 antibody (e.g., daratumumab) and about 30,000 U of the hyaluronidase (e.g., rHuPH20 recombinant hyaluronidase) is administered on 28-day cycles, once weekly during Cycle 1 (e.g., on Days 1, 8, 15 and 22), every 2 weeks during Cycles 2-5 (e.g., on Days 1 and 15), and every 4 weeks (e.g., on Day 1) thereafter.

In some embodiments, the dosing regimen of the method results in a reduction, elimination or reduction and elimination of corticosteroid use by the subject.

### Corticosteroid Tapering

In one aspect, the disclosure provides a method of treating a hematologic malignancy (e.g., a CD38-positive hematologic malignancy such as MM), comprising administering to a subject in need thereof a therapeutically effective amount of an anti-CD38 antibody and a corticosteroid for a time sufficient to treat the hematologic malignancy, wherein the dosing regimen includes a reduction, elimination, or reduction followed by elimination, of corticosteroid administration to the subject.

The anti-CD38 antibody can be any one of the anti-CD38 antibodies described herein.

Non-limiting examples of corticosteroid include bethamethasone, cortisol, cortisone, dexamethasone, glucocorticoid, hydrocortisone, methylprednisolone (MP), prednisolone, prednisone and triamcinolone. In some embodiments, corticosteroid refers to a class of steroid hormones that are produced in the adrenal cortex or produced synthetically. In some embodiments, corticosteroid comprises, consists essentially of, or consists of dexamethasone, methylprednisolone, prednisolone, prednisone.

In some embodiments, the corticosteroid is administered concomitantly with administration (e.g., subcutaneous administration) of the anti-CD38 antibody.

The corticosteroid can be administered by any suitable method known in the art.

In some embodiments, the corticosteroid is administered orally.

In some embodiments, the corticosteroid is administered parenterally (e.g., intravenously).

In some embodiments, the corticosteroid is re-administered concomitantly with the administration of the anti-CD38 antibody.

In some embodiments, the corticosteroid is re-administered subsequent to (e.g., just after) the administration of the anti-CD38 antibody.

In some embodiments, the corticosteroid is re-administered about 1 minute to about 15 minutes subsequent to the administration of the anti-CD38 antibody.

In some embodiments, the corticosteroid is re-administered about 0.5 hour to about 10 hours subsequent to the administration of the anti-CD38 antibody, e.g., about: 1-10, 2-10, 4-10, 6-10 or 7-9 hours subsequent to the administration of the anti-CD38 antibody.

In some embodiments, the corticosteroid is re-administered about: 0.5, 1, 2, 3, 4, 5, 6, 7, 8, 9 or 10 hours subsequent to the administration of the anti-CD38 antibody.

In some embodiments, the corticosteroid administered prior to administration of the anti-CD38 antibody and the corticosteroid re-administered subsequent to administration of the anti-CD38 antibody are the same.

In some embodiments, the corticosteroid administered prior to administration of the anti-CD38 antibody and the corticosteroid re-administered subsequent to administration of the anti-CD38 antibody are different.

In some embodiments, prednisone is orally administered about 1 hour to about 2 hours prior to subcutaneous administration of the anti-CD38 antibody, and is orally re-administered about 7 to 9 hours post-administration of the anti-CD38 antibody.

In some embodiments, the anti-CD38 antibody is administered once weekly, every 2 weeks, or every 4 weeks during a 28-day cycle. In certain embodiments, the anti-CD38 antibody is administered once weekly during Cycle 1, every 2 weeks during Cycles 2-5, and every 4 weeks thereafter.

In some embodiments, the corticosteroid administered to the subject is reduced by at least about 20% (e.g., during a 28-day treatment cycle), for example, reduced by at least about: 25%, 30%, 35%, 40%, 45%, 50%, 55%, 60%, 65%, 70%, 75%, 80%, 85%, 90% or 95% (e.g., during a 28-day treatment cycle). In some embodiments, the corticosteroid administered to the subject is reduced by about 20-95% (e.g., during a 28-day treatment cycle), for example, reduced by about: 25-95%, 25-90%, 30-90%, 30-85%, 35-85%, 35-80%, 40-80%, 40-75%, 45-75%, 45-70%, 50-70%, 55-65% or 55-60% (e.g., during a 28-day treatment cycle). In some embodiments, the corticosteroid administered to the subject is reduced by about 60%. In certain embodiments, the corticosteroid is eliminated. In particular embodiments, the corticosteroid administered to the subject is reduced by about 60% and then eliminated during a 28-day treatment cycle.

In some embodiments, the corticosteroid administered to the subject is administered at least once, twice, three times, four times, five times or six times, and then eliminated during a 28-day treatment cycle. In some embodiments, the corticosteroid administered to the subject is administered once and then eliminated during a 28-day treatment cycle.

In some embodiments, the method further comprises administering to the subject a therapy on a 28-day cycle comprising:
a) administering about 1,800 mg anti-CD38 antibody and about 30,000 U hyaluronidase subcutaneously on days 1, 8, 15 and 22; and
b) administering about 20 mg pre-dose corticosteroid intravenously on day 1, of the 28-day cycle.

In some embodiments, the method further comprises administering to the subject a therapy on a 28-day cycle comprising:

administering about 1,800 mg anti-CD38 antibody and about 30,000 U rHuPH20 hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8; and

administering about 20 mg post-dose corticosteroid on day 8, of the 28-day cycle.

In some embodiments, the method further comprises administering to the subject a therapy on a 28-day cycle comprising:

administering about 1,800 mg anti-CD38 antibody and about 30,000 U hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8;

administering about 20 mg post-dose corticosteroid on day 8;

administering about 30 mg pre-dose corticosteroid on day 15; and

administering about 20 mg post-dose corticosteroid on day 15, of the 28-day cycle.

In another aspect, the disclosure provides a method of treating hematologic malignancy in a subject in need thereof, the method comprising administering to the subject a therapy on a 28-day cycle, wherein the therapy comprises:
a) administering about 1,800 mg of the anti-CD38 antibody and about 30,000 U on days 1, 8, 15 and 22; and
b) administering about 20 mg (or equivalent) pre-dose corticosteroid on day 1, of the 28-day cycle.

In another aspect, the disclosure provides a method of treating hematologic malignancy in a subject in need thereof, the method comprising administering to the subject a therapy on a 28-day cycle, wherein the therapy comprises:

administering about 1,800 mg anti-CD38 antibody and about 30,000 U rHuPH20 hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8; and

administering about 20 mg post-dose corticosteroid on day 8, of the 28-day cycle.

In another aspect, the disclosure provides a method of treating hematologic malignancy in a subject in need thereof, the method comprising administering to the subject a therapy on a 28-day cycle, wherein the therapy comprises:

administering about 1,800 mg anti-CD38 antibody and about 30,000 U hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8;

administering about 20 mg post-dose corticosteroid on day 8;

administering about 30 mg pre-dose corticosteroid on day 15; and

administering about 20 mg post-dose corticosteroid on day 15, of the 28-day cycle.

### Dexamethasone

In some embodiments, the corticosteroid comprises, consists essentially of or consists of dexamethasone. Dexamethasone is marketed under the trade name DECARON®.

Daratumumab in combination with dexamethasone is indicated for the treatment of adult patients with multiple myeloma or light chain amyloidosis. “In combination with” means that two or more therapeutics are administered to a subject together in a mixture, concurrently as single agents or sequentially as single agents in any order. For example, in combination with lenalidomide and dexamethasone in newly diagnosed multiple myeloma patients who are ineligible for autologous stem cell transplant and in patients with relapsed or refractory multiple myeloma who have received at least one prior therapy; in combination with bortezomib, thalidomide, and dexamethasone in newly diagnosed multiple myeloma patients who are eligible for autologous stem cell transplant; in combination with bortezomib and dexamethasone in multiple myeloma patients who have received at least one prior therapy; in combination with carfilzomib and dexamethasone in multiple myeloma patients who have received one to three prior lines of therapy; in combination with pomalidomide and dexamethasone in multiple myeloma patients who have received at least two prior therapies including lenalidomide and a proteasome inhibitor; and in newly diagnosed light chain amyloidosis patients in combination with bortezomib, cyclophosphamide and dexamethasone. Additional information regarding daratumumab in combination with dexamethasone can be found, for example, in the prescribing information product insert for DARZALEX® (www_janssenlabels_com/package-insert/product-monograph/prescribing-information/DARZALEX-pi.pdf), which is incorporated herein by reference.

In some embodiments, the dexamethasone administered (e.g., orally or intravenously) to the subject is reduced by at least about 20% (e.g., during a 28-day treatment cycle), for example, reduced by at least about: 25%, 30%, 35%, 40%, 45%, 50%, 55%, 60%, 65%, 70%, 75%, 80%, 85%, 90% or 95% (e.g., during a 28-day treatment cycle). In some embodiments, the dexamethasone administered to the subject is reduced by about 20-95% (e.g., during a 28-day treatment cycle), for example, reduced by about: 25-95%, 25-90%, 30-90%, 30-85%, 35-85%, 35-80%, 40-80%, 40-75%, 45-75%, 45-70%, 50-70%, 55-65% or 55-60% (e.g., during a 28-day treatment cycle). In some embodiments, the dexamethasone administered to the subject is reduced by at least about 60%. In certain embodiments, the dexamethasone is eliminated. In particular embodiments, the dexamethasone administered to the subject is reduced by at least about 60% and then eliminated during a 28-day treatment cycle.

In some embodiments, the dexamethasone administration prior to the anti-CD38 antibody administration (e.g., DARZALEX FASPRO® administration) is reduced.

In some embodiments, the dexamethasone administered to the subject is less than about 20 mg (or equivalent) (e.g., during a 28-day treatment cycle), for example, less than about: 19, 18, 17, 16, 15, 14, 13, 12, 11, 10, 9, 8, 7, 6, 5, 4, 3, 2 or 1 mg (or equivalent) (e.g., during a 28-day treatment cycle). In some embodiments, the dexamethasone administered to the subject is about 0-20 mg (or equivalent) (e.g., during a 28-day treatment cycle), for example, about: 0-19, 1-19, 1-18, 2-18, 2-17, 3-17, 3-16, 4-16, 4-15, 5-15, 5-14, 6-14, 6-13, 7-13, 7-12, 8-12, 8-11, 9-11 or 9-10 mg (or equivalent) (e.g., during a 28-day treatment cycle). In some embodiments, the dexamethasone administered to the subject is less than about 8 mg (or equivalent) (e.g., during a 28-day treatment cycle). In certain embodiments, the dexamethasone is eliminated. In particular embodiments, the dexamethasone administered to the subject is less than about 8 mg (or equivalent) and then eliminated during a 28-day treatment cycle.

In some embodiments, the dexamethasone administration subsequent to the anti-CD38 antibody administration (e.g., DARZALEX FASPRO® administration) is reduced.

In some embodiments, the method comprises administering to the subject a therapy on a 28-day cycle, wherein the therapy comprises:
a) administering about 1,800 mg of daratumumab and about 30,000 U rHuPH20 recombinant hyaluronidase subcutaneously on days 1, 8, 15 and 22; and
b) administering about 20 mg (or equivalent) pre-dose dexamethasone intravenously on day 1, of the 28-day cycle.

In another aspect, the disclosure provides a method of treating hematologic malignancy in a subject in need thereof, the method comprising administering to the subject a therapy on a 28-day cycle, wherein the therapy comprises:
a) administering about 1,800 mg of the anti-CD38 antibody and about 30,000 U on days 1, 8, 15 and 22; and
b) administering about 20 mg (or equivalent) pre-dose dexamethasone on day 1, of the 28-day cycle.

### Methylprednisolone (MP)

In some embodiments, the corticosteroid is methylprednisolone (MP). For administration of MP, as a pre- or post-medication, see, e.g., the prescribing information product insert for DARZALEX®.

In some embodiments, the MP administered (e.g., orally or intravenously) to the subject is reduced by at least about 20% (e.g., during a 28-day treatment cycle), for example, reduced by at least about: 25%, 30%, 35%, 40%, 45%, 50%, 55%, 60%, 65%, 70%, 75%, 80%, 85%, 90% or 95% (e.g., during a 28-day treatment cycle). In some embodiments, the MP administered to the subject is reduced by about 20-95% (e.g., during a 28-day treatment cycle), for example, reduced by about: 25-95%, 25-90%, 30-90%, 30-85%, 35-85%, 35-80%, 40-80%, 40-75%, 45-75%, 45-70%, 50-70%, 55-65% or 55-60% (e.g., during a 28-day treatment cycle). In some embodiments, the MP administered to the subject is reduced by at least about 60%. In certain embodiments, the MP is eliminated. In particular embodiments, the MP administered to the subject is reduced by at least about 60% and then eliminated during a 28-day treatment cycle.

In some embodiments, the MP administration prior to the anti-CD38 antibody administration (e.g., DARZALEX FASPRO® administration) is reduced.

In some embodiments, the MP administered to the subject prior to the anti-CD38 antibody administration is less than about 100 mg (or equivalent) (e.g., during a 28-day treatment cycle), for example, less than about: 90, 80, 70, 60, 50, 40, 35, 30, 25, 20, 15, 10, 5, 4, 3, 2 or 1 mg (or equivalent) (e.g., during a 28-day treatment cycle). In some embodiments, the MP administered to the subject prior to the anti-CD38 antibody administration is about 0-100 mg (or equivalent) (e.g., during a 28-day treatment cycle), for example, about: 0-90, 1-90, 1-80, 2-80, 2-70, 3-70, 3-60, 4-60, 4-50, 5-50, 5-40, 10-40, 10-35, 15-35, 15-30, 20-30 or 20-25 mg (or equivalent) (e.g., during a 28-day treatment cycle). In some embodiments, the MP administered to the subject prior to the anti-CD38 antibody administration is less than about 40 mg (or equivalent) (e.g., during a 28-day treatment cycle). In certain embodiments, the MP administered to the subject prior to the anti-CD38 antibody administration is eliminated. In particular embodiments, the MP administered to the subject prior to the anti-CD38 antibody administration is less than about 40 mg (or equivalent) and then eliminated during a 28-day treatment cycle.

In some embodiments, the MP administered to the subject prior to the anti-CD38 antibody administration is less than about 60 mg (or equivalent) (e.g., during a 28-day treatment cycle), for example, less than about: 55, 50, 45, 40, 35, 30, 25, 20, 15, 10, 5, 4, 3, 2 or 1 mg (or equivalent) (e.g., during a 28-day treatment cycle). In some embodiments, the MP administered to the subject prior to the anti-CD38 antibody administration is about 0-60 mg (or equivalent) (e.g., during a 28-day treatment cycle), for example, about: 0-55, 1-55, 1-50, 2-50, 2-45, 3-45, 3-40, 4-40, 4-35, 5-35, 5-30, 10-30, 10-25, 15-25 or 15-20 mg (or equivalent) (e.g., during a 28-day treatment cycle). In some embodiments, the MP administered to the subject prior to the anti-CD38 antibody administration is less than about 24 mg (or equivalent) (e.g., during a 28-day treatment cycle). In certain embodiments, the MP administered to the subject prior to the anti-CD38 antibody administration is eliminated. In particular embodiments, the MP administered to the subject prior to the anti-CD38 antibody administration is less than about 24 mg (or equivalent) and then eliminated during a 28-day treatment cycle.

In some embodiments, the MP administered to the subject prior to the anti-CD38 antibody administration is less than about 30 mg (or equivalent) (e.g., during a 28-day treatment cycle), for example, less than about: 25, 20, 18, 15, 10, 9, 8, 7, 6, 5, 4, 3, 2 or 1 mg (or equivalent) (e.g., during a 28-day treatment cycle). In some embodiments, the MP administered to the subject prior to the anti-CD38 antibody administration is about 0-30 mg (or equivalent) (e.g., during a 28-day treatment cycle), for example, about: 0-25, 1-25, 1-20, 2-20, 2-18, 3-18, 3-15, 4-15, 4-10, 5-10, 5-9, 6-9, 6-8 or 7-8 mg (or equivalent) (e.g., during a 28-day treatment cycle). In some embodiments, the MP administered to the subject prior to the anti-CD38 antibody administration is less than about 12 mg (or equivalent) (e.g., during a 28-day treatment cycle). In certain embodiments, the MP administered to the subject prior to the anti-CD38 antibody administration is eliminated. In particular embodiments, the MP administered to the subject prior to the anti-CD38 antibody administration is less than about 12 mg (or equivalent) and then eliminated during a 28-day treatment cycle.

In some embodiments, the MP administration subsequent to the anti-CD38 antibody administration (e.g., DARZALEX FASPRO® administration) is reduced.

In some embodiments, the MP administered to the subject subsequent to the anti-CD38 antibody administration is less than about 20 mg (or equivalent) (e.g., during a 28-day treatment cycle), for example, less than about: 19, 18, 17, 16, 15, 14, 13, 12, 11, 10, 9, 8, 7, 6, 5, 4, 3, 2 or 1 mg (or equivalent) (e.g., during a 28-day treatment cycle). In some embodiments, the MP administered to the subject subsequent to the anti-CD38 antibody administration is about 0-20 mg (or equivalent) (e.g., during a 28-day treatment cycle), for example, about: 0-19, 1-19, 1-18, 2-18, 2-17, 3-17, 3-16, 4-16, 4-15, 5-15, 5-14, 6-14, 6-13, 7-13, 7-12, 8-12, 8-11, 9-11 or 9-10 mg (or equivalent) (e.g., during a 28-day treatment cycle). In some embodiments, the MP administered to the subject subsequent to the anti-CD38 antibody administration is less than about 8 mg (or equivalent) (e.g., during a 28-day treatment cycle). In certain embodiments, the MP administered to the subject subsequent to the anti-CD38 antibody administration is eliminated. In particular embodiments, the MP administered to the subject subsequent to the anti-CD38 antibody administration is less than about 8 mg (or equivalent) and then eliminated during a 28-day treatment cycle.

In some embodiments, both the MP administration prior to the anti-CD38 antibody administration and the MP administration subsequent to the anti-CD38 antibody administration are reduced.

In some embodiments, the method comprises administering to the subject a therapy on a 28-day cycle, wherein the therapy comprises:

administering about 1,800 mg anti-CD38 antibody and about 30,000 U rHuPH20 hyaluronidase on days 1, 8, 15 and 22;

administering about 100 mg pre-dose MP on day 1;

administering about 20 mg post-dose MP on days 1 and 2;

administering about 60 mg pre-dose MP on day 8; and

administering about 20 mg post-dose MP on day 8, of the 28-day cycle.

In some embodiments, the method comprises administering to the subject a therapy on a 28-day cycle, wherein the therapy comprises:

administering about 1,800 mg daratumumab and about 30,000 U rHuPH20 recombinant hyaluronidase subcutaneously on days 1, 8, 15 and 22;

administering about 100 mg pre-dose MP orally or intravenously on day 1;

administering about 20 mg post-dose MP orally on days 1 and 2;

administering about 60 mg pre-dose MP orally or intravenously on day 8;

administering about 20 mg post-dose MP orally on day 8;

administering about 30 mg pre-dose MP orally or intravenously on day 15; and

administering about 20 mg post-dose MP orally on day 15, of the 28-day cycle.

### Prednisone

In some embodiments, the corticosteroid is prednisone.

Daratumumab in combination with prednisone is indicated for the treatment of adult patients with multiple myeloma. For example, in combination with bortezomib, melphalan and prednisone in newly diagnosed patients who are ineligible for autologous stem cell transplant. Additional information regarding daratumumab in combination with prednisone can be found, for example, in the prescribing information product insert for DARZALEX®.

In some embodiments, the prednisone administered (e.g., orally) to the subject is reduced by at least about 20% (e.g., during a 28-day treatment cycle), for example, reduced by at least about: 25%, 30%, 35%, 40%, 45%, 50%, 55%, 60%, 65%, 70%, 75%, 80%, 85%, 90% or 95% (e.g., during a 28-day treatment cycle). In some embodiments, the prednisone administered to the subject is reduced by about 20-95% (e.g., during a 28-day treatment cycle), for example, reduced by about: 25-95%, 25-90%, 30-90%, 30-85%, 35-85%, 35-80%, 40-80%, 40-75%, 45-75%, 45-70%, 50-70%, 55-65% or 55-60% (e.g., during a 28-day treatment cycle). In some embodiments, the prednisone administered to the subject is reduced by at least about 60%. In certain embodiments, the prednisone administered to the subject is eliminated. In particular embodiments, the prednisone administered to the subject is reduced by at least about 60% and then eliminated during a 28-day treatment cycle.

In some embodiments, the prednisone administration subsequent to the anti-CD38 antibody administration (e.g., DARZALEX FASPRO® administration) is reduced.

In some embodiments, the prednisone administered to the subject subsequent to the anti-CD38 antibody administration is less than about 60 mg/m² (or equivalent) (e.g., during a 28-day treatment cycle), for example, less than about: 55, 50, 45, 40, 35, 30, 25, 20, 15, 10, 5, 4, 3, 2 or 1 mg/m² (or equivalent) (e.g., during a 28-day treatment cycle). In some embodiments, the prednisone administered to the subject subsequent to the anti-CD38 antibody administration is about 0-60 mg/m² (or equivalent) (e.g., during a 28-day treatment cycle), for example, about: 0-55, 1-55, 1-50, 2-50, 2-45, 3-45, 3-40, 4-40, 4-35, 5-35, 5-30, 10-30, 10-25, 15-25 or 15-20 mg/m² (or equivalent) (e.g., during a 28-day treatment cycle). In some embodiments, the prednisone administered to the subject subsequent to the anti-CD38 antibody administration is less than about 24 mg/m² (or equivalent) (e.g., during a 28-day treatment cycle). In certain embodiments, the prednisone administered to the subject is eliminated. In particular embodiments, the prednisone administered to the subject subsequent to the anti-CD38 antibody administration is less than about 24 mg/m² (or equivalent) and then eliminated during a 28-day treatment cycle.

In some embodiments, the prednisone administration prior to the anti-CD38 antibody administration (e.g., DARZALEX FASPRO® administration) is reduced.

In some embodiments, the prednisone administered to the subject prior to the anti-CD38 antibody administration is less than about 60 mg/m² (or equivalent) (e.g., during a 28-day treatment cycle), for example, less than about: 55, 50, 45, 40, 35, 30, 25, 20, 15, 10, 5, 4, 3, 2 or 1 mg/m² (or equivalent) (e.g., during a 28-day treatment cycle). In some embodiments, the prednisone administered to the subject prior to the anti-CD38 antibody administration is about 0-60 mg/m² (or equivalent) (e.g., during a 28-day treatment cycle), for example, about: 0-55, 1-55, 1-50, 2-50, 2-45, 3-45, 3-40, 4-40, 4-35, 5-35, 5-30, 10-30, 10-25, 15-25 or 15-20 mg/m² (or equivalent) (e.g., during a 28-day treatment cycle). In some embodiments, the prednisone administered to the subject prior to the anti-CD38 antibody administration is less than about 24 mg/m² (or equivalent) (e.g., during a 28-day treatment cycle). In certain embodiments, the prednisone is eliminated. In particular embodiments, the prednisone administered to the subject prior to the anti-CD38 antibody administration is less than about 24 mg/m² (or equivalent) and then eliminated during a 28-day treatment cycle.

In some embodiments, both the prednisone administration prior to the anti-CD38 antibody administration and the prednisone administration subsequent to the anti-CD38 antibody administration are reduced.

### “Cortisone-Free” Treatment

In another aspect, the disclosure provides a method of treating a hematologic malignancy, wherein the method comprises administering to a subject in need thereof a therapeutically effective amount of an anti-CD38 antibody for a time sufficient to treat the hematologic malignancy, without co-administering a corticosteroid.

In another aspect, the disclosure provides a method of treating a hematologic malignancy, comprising administering to a subject in need thereof a therapeutically effective amount of an anti-CD38 antibody and a corticosteroid dose of <2.0 mg/kg/day or equivalent for a time sufficient to treat the hematologic malignancy.

In another aspect, the disclosure provides a method of treating a hematologic malignancy, comprising administering to a subject in need thereof a therapeutically effective amount of an anti-CD38 antibody for a time sufficient to treat the hematologic malignancy, wherein disease control or complete remission is achieved and/or maintained at a corticosteroid dose of <2.0 mg/kg/day or equivalent.

Non-limiting examples of corticosteroid include bethamethasone, cortisol, cortisone, dexamethasone, glucocorticoid, hydrocortisone, methylprednisolone (MP), prednisolone, prednisone and triamcinolone. In some embodiments, corticosteroid refers to a class of steroid hormones that are produced in the adrenal cortex or produced synthetically. In some embodiments, corticosteroid comprises, consists essentially of, or consists of dexamethasone, methylprednisolone, prednisolone, prednisone.

In some embodiments, the corticosteroid dose is of less than about: 2.0, 1.9, 1.8, 1.7, 1.6, 1.5, 1.4, 1.3, 1.2, 1.1, 1.0, 0.8, 0.5, 0.2, 0.1, 0.08, 0.05, 0.02, 0.01, 0.008, 0.005, 0.002 or 0.001 mg/kg/day or equivalent.

In some embodiments, the corticosteroid dose is of less than about: 2.0, 1.9, 1.8, 1.7, 1.6, 1.5, 1.4, 1.3, 1.2, 1.1, 1.0, 0.8, 0.5, 0.2, 0.1, 0.08, 0.05, 0.02, 0.01, 0.008, 0.005, 0.002 or 0.001 mg/m²/day or equivalent.

In some embodiments, the method further comprises administering to the subject a prior therapy. In some embodiments, the prior therapy has a 28-day cycle.

In some embodiments, the prior therapy has a 28-day cycle, comprising:
a) administering about 1,800 mg daratumumab and about 30,000 U rHuPH20 recombinant hyaluronidase subcutaneously on days 1, 8, 15 and 22; and
b) administering about 20 mg pre-dose dexamethasone intravenously on day 1, of the 28-day cycle.

In certain embodiments, the prior therapy has a 28-day cycle, comprising:

administering about 1,800 mg daratumumab and about 30,000 U rHuPH20 recombinant hyaluronidase subcutaneously on days 1, 8, 15 and 22;

administering about 100 mg pre-dose methylprednisolone (MP) orally or intravenously on day 1;

administering about 20 mg post-dose MP orally on days 1 and 2;

administering about 60 mg pre-dose MP orally or intravenously on day 8; and

administering about 20 mg post-dose MP orally on day 8, of the 28-day cycle.

In particular embodiments, the prior therapy has a 28-day cycle, comprising:

administering about 1,800 mg daratumumab and about 30,000 U rHuPH20 recombinant hyaluronidase subcutaneously on days 1, 8, 15 and 22;

administering about 100 mg pre-dose methylprednisolone (MP) orally or intravenously on day 1;

administering about 20 mg post-dose MP orally on days 1 and 2;

administering about 60 mg pre-dose MP orally or intravenously on day 8;

administering about 20 mg post-dose MP orally on day 8;

administering about 30 mg pre-dose MP orally or intravenously on day 15; and

administering about 20 mg post-dose MP orally on day 15, of the 28-day cycle.

In some embodiments, the corticosteroid is administered prior to (e.g., just prior to) administration (e.g., subcutaneous administration) of the anti-CD38 antibody.

In some embodiments, the corticosteroid is administered about 1 minute to about 5 hours prior to administration of the anti-CD38 antibody, e.g., about: 1-15 minutes, 5-15 minutes, 10-15 minutes, 0.5-5 hours, 0.5-4 hours, 1-4 hours or 1-2 hours prior to administration (e.g., subcutaneous administration) of the anti-CD38 antibody. In certain embodiments, the corticosteroid is administered about: 0.5, 1, 1.5, 2, 2.5, 3, 3.5, 4, 4.5 or 5 hours prior to administration (e.g., subcutaneous administration) of the anti-CD38 antibody.

In some embodiments, the corticosteroid comprises or consists of prednisone. In some embodiments, prednisone is administered the day after the administration (e.g., subcutaneous administration) of the anti-CD38 antibody in the prior therapy.

### Hematologic Malignancy

In some embodiments, the hematologic malignancy is a CD38-positive hematologic malignancy. “CD38-positive hematologic malignancy” refers to a hematologic malignancy characterized by the presence of tumor cells expressing CD38 including leukemias, lymphomas and myeloma. Non-limiting examples of such CD38-positive hematologic malignancies include precursor B-cell lymphoblastic leukemia/lymphoma and B-cell non-Hodgkin's lymphoma, acute promyelocytic leukemia, acute lymphoblastic leukemia and mature B-cell neoplasms, such as B-cell chronic lymphocytic leukemia(CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma, plasma cell leukemia, post-transplant lymphoproliferative disorder, light chain amyloidosis, Waldenstrom's macroglobulinemia, plasma cell leukemias, and anaplastic large-cell lymphoma (ALCL).

In some embodiments, the CD38-positive hematologic malignancy is a plasma cell disease. In some embodiments, the CD38-positive hematologic malignancy is multiple myeloma (MM), acute lymphoblastic leukemia, diffuse large B-cell lymphoma, Burkitt's lymphoma, follicular lymphoma, mantle-cell lymphoma, acute myeloid leukemia, chronic lymphocytic leukemia, a smoldering multiple myeloma (SMM), or a combination thereof. In certain embodiments, the plasma cell disease is light chain amyloidosis, MM or Waldenstrom's macroglobulinemia.

In particular embodiments, the plasma cell disease is MM. In some embodiments, the MM is light chain MM (LCMM), non-secretory MM (NSMM), solitary plasmacytoma (SP), extramedullary plasmacytoma (EMP), monoclonal gammopathy of undetermined significance (MGUS), smoldering MM (SMM), Immunoglobulin D MM (IgD MM) or Immunoglobulin E (IgE) MM, or a combination thereof. In some embodiments, the MM is newly diagnosed MM (NDMM). In some embodiments, the MM is relapsed or refractory MM (RRMM).

In some embodiments, the hematologic malignancy is a hematologic malignancy. The term “cancer” refers to a disease caused by an uncontrolled division of cells in a part of the body.

### Additional Therapeutic Agents

In some embodiments, the method further comprises administering to the subject one or more additional therapeutic agents.

In some embodiments, the anti-CD38 antibody and the one or more additional therapeutic agents are administered simultaneously. In other embodiments, the anti-CD38 antibody and the one or more additional therapeutic agents are administered separately (e.g., sequentially).

In some embodiments, the one or more additional therapeutic agents comprise a T cell expressing chimeric antigen receptor (CAR) (CAR-T cell), a natural killer cell expressing CAR (CAR-NK cell), a macrophage expressing CAR (CAR-M cell), a chemotherapeutic agent, a bispecific antibody, an immune checkpoint inhibitor, a T-cell redirector, radiation therapy, surgery, standard of care drug or a combination thereof.

### T Cells Expressing Chimeric Antigen Receptor (CAR) (CAR-T Cells)

In some embodiments, the one or more additional therapeutic agents comprise a T cell expressing chimeric antigen receptor (CAR) (CAR-T cell). CAR T cells are described in International Application No. PCT/IB2018/001619, the contents of which are incorporated herein by reference.

In some embodiments, the CAR comprises an extracellular antigen-binding domain, a transmembrane domain and an intracellular signaling domain.

In one embodiment, the intracellular signaling domain comprises a T-cell surface glycoprotein CD3 zeta chain component.

In some embodiments, the extracellular antigen-binding domain binds G-protein coupled receptor family C group 5 member D (GPRC5D). The term “G-protein coupled receptor family C group 5 member D” and “GPRC5D” specifically include the human GPRC5D protein, for example as described in GenBank Accession No. BC069341, NCBI Reference Sequence: NP 061124.1 and UniProtKB/Swiss-Prot Accession No. Q9NZD1 (see also Brauner-Osborne et al., Biochim Biophys Acta. 1518(3):237-48 (2001)). The term “GPRC5D” includes proteins comprising mutations, e.g., point mutations, fragments, insertions, deletions and splice variants of full length wild type human GPRC5D. The term “GPRC5D” also encompasses post-translational modifications of the GPRC5D amino acid sequence. Post-translational modifications include, but are not limited to, N- and O-linked glycosylation.

In some embodiments, the extracellular antigen-binding domain binds GPRC5D and CD3. In some embodiments, the one or more additional therapeutic agents comprise an anti-GPRC5D CAR-T and/or an anti-GPRC5D CAR-NK, or a combination thereof. Non-limiting examples of CARs targeting GPRC5D can be found in WO2016090312 and WO2020148677, the contents of which are incorporated herein by reference.

In some embodiments, the extracellular antigen-binding domain binds B cell maturation antigen (BCMA). The human and murine amino acid and nucleic acid sequences of BCMA can be found in a public database (e.g., GenBank, UniProt, or Swiss-Prot). See, e.g., UniProt/Swiss-Prot Accession Nos. Q02223 (human BCMA) and 088472 (murine BCMA).

In some embodiments, the extracellular antigen-binding domain binds BCMA and CD3. In some embodiments, the one or more additional therapeutic agents comprise an anti-BCMA CAR-T and/or an anti-BCMA CAR-NK, or a combination thereof. Non-limiting examples of CARs targeting BCMA can be found in WO2013154760, WO2016014789, WO2016094304, WO2017025038 and WO2018028647, the contents of which are incorporated herein by reference.

### T-Cell Redirector

In some embodiments, the T-cell redirector comprises a soluble bispecific antibody (bsAb) or a membrane-anchored chimeric antigen receptor, or a combination thereof.

In some embodiments, the soluble bispecific antibody binds GPRC5D and CD3. Non-limiting examples of bispecific antigen binding molecules that bind GPRC5D and CD3 can be found in WO2018017786, the contents of which are incorporated herein by reference.

In some embodiments, the soluble bispecific antibody binds BCMA and CD3. Non-limiting examples of bispecific antigen binding molecules that bind BCMA and CD3 can be found in WO2017031104, the contents of which are incorporated herein by reference.

### Immune Checkpoint Inhibitors

In some embodiments, the one or more additional therapeutic agents comprise an immune checkpoint inhibitor.

In some embodiments, the immune checkpoint inhibitor is an anti-PD-1 antibody, an anti-PD-L1 antibody, an anti-PD-L2 antibody, an anti-LAG3 antibody, an anti-TIM3 antibody, or an anti-CTLA-4 antibody.

In some embodiments, the immune checkpoint inhibitor is an anti-PD-1 antibody. In some embodiments, the anti-PD-1 antibody comprises a VH and VL amino acid sequences of:
a) SEQ ID NO: 23 and SEQ ID NO: 24, respectively;
b) SEQ ID NO: 25 and SEQ ID NO: 26, respectively;
c) SEQ ID NO: 33 and SEQ ID NO: 34, respectively; or
d) SEQ ID NO: 35 and SEQ ID NO:36, respectively.

In some embodiments, the immune checkpoint inhibitor is an anti-PD-L1 antibody. In some embodiments, the anti-PD-L1 antibody comprises a VH and VL amino acid sequences of:
a) SEQ ID NO: 27 and SEQ ID NO: 28, respectively;
b) SEQ ID NO: 29 and SEQ ID NO: 30, respectively; or
c) SEQ ID NO: 31 and SEQ ID NO: 32, respectively.

In some embodiments, the immune checkpoint inhibitor is an anti-PD-L2 antibody.

In some embodiments, the immune checkpoint inhibitor is an anti-LAG3 antibody. Non-limiting examples of anti-LAG-3 antibodies include those described in Int. Pat. Publ. No. WO2010/019570.

In some embodiments, the immune checkpoint inhibitor is an anti-TIM-3 antibody. In some embodiments, the anti-TIM-3 antibody comprises a VH and VL amino acid sequences of:
a) SEQ ID NO: 37 and SEQ ID NO: 38, respectively; or
b) SEQ ID NO: 39 and SEQ ID NO: 40, respectively.

In some embodiments, the immune checkpoint inhibitor is an anti-CTLA-4 antibody. A non-limiting example of anti-CTLA-4 antibodies is Ipilimumab.

The anti-PD-1, anti-PD-L1, anti-PD-L2, anti-LAG3, anti-TIM3 and anti-CTLA-4 antibodies may be generated de novo.

In some embodiments, the anti-CD38 antibody and the immune checkpoint inhibitor are administered simultaneously. In some embodiments, the anti-CD38 antibody and the immune checkpoint inhibitor are administered sequentially or separately.

### Hematopoietic Stem Cell Transplantation (HSCT)

In some embodiments, the one or more additional therapeutic agents comprise a hematopoietic stem cell transplantation (HSCT). “Hematopoietic stem cell transplantation” is the transplantation of blood stem cells derived from the bone marrow (in this case known as bone marrow transplantation), blood (such as peripheral blood and umbilical cord blood), or amniotic fluid. Undergoing hematopoietic stem cell transplantation” means that the patient did already receive, is receiving or will receive HSCT.

In some embodiments, the HSCT is allogeneic. In some embodiments, the HSCT is autologous or syngeneic (i.e., the donor is a twin). Autologous HSCT comprises the extraction of HSC from the subject and freezing of the harvested HSC. After myeloablation, the subject's stored HSC are transplanted into the subject. Allogeneic HSCT involves HSC obtained from an allogeneic HSC donor who has an HLA type that matches the subject.

In some embodiments, the subject has completed chemotherapy and/or radiation therapy prior to HSCT.

Patients may be treated with chemotherapy and/or radiation therapy prior to HSCT (so-called pre-transplant preparation) to eradicate some or all of the patient's hematopoietic cells prior to transplant. The patient may also be treated with immunosuppressants in case of allogeneic HSCT. An exemplary pre-transplant preparation therapy is high-dose melphalan (see, e.g., Skinner et al., Ann. Intern. Med. 140:85-93 (2004), Gertz et al., Bone Marrow Transplant 34:1025-31 (2004), Perfetti et al., Haematologica 91:1635-43 (2006)). The radiation therapy that may be employed in pre-transplant treatment may be carried out according to protocols commonly known in this field. Radiation therapy may be provided simultaneously, sequentially or separately with the anti-CD38 antibody.

### Radiation Therapy

In some embodiments, the method of the invention further comprises administering a form of radiation therapy, surgery or a combination thereof. Non-limiting examples of radiation therapies include external beam radiation, intensity modulated radiation therapy (IMRT), focused radiation, and any form of radiosurgery including Gamma Knife, Cyberknife, Linac, and interstitial radiation (e.g., implanted radioactive seeds, GliaSite balloon).

Focused radiation methods that may be used include stereotactic radiosurgery, fractionated stereotactic radiosurgery, and intensity-modulated radiation therapy (IMRT). It is apparent that stereotactic radiosurgery involves the precise delivery of radiation to a tumorous tissue, for example, a brain tumor, while avoiding the surrounding nontumorous, normal tissue. The dosage of radiation applied using stereotactic radiosurgery may vary, typically from 1 Gy to about 30 Gy, and may encompass intermediate ranges including, for example, from 1 to 5, 10, 15, 20, 25, up to 30 Gy in dose. Because of noninvasive fixation devices, stereotactic radiation need not be delivered in a single treatment. The treatment plan may be reliably duplicated day-to-day, thereby allowing multiple fractionated doses of radiation to be delivered. When used to treat a tumor over time, the radiosurgery is referred to as “fractionated stereotactic radiosurgery” or FSR. In contrast, stereotactic radiosurgery refers to a one-session treatment. Fractionated stereotactic radiosurgery may result in a high therapeutic ratio, i.e., a high rate of killing of tumor cells and a low effect on normal tissue. The tumor and the normal tissue respond differently to high single doses of radiation vs. multiple smaller doses of radiation. Single large doses of radiation may kill more normal tissue than several smaller doses of radiation may. Accordingly, multiple smaller doses of radiation can kill more tumor cells while sparing normal tissue. The dosage of radiation applied using fractionated stereotactic radiation may vary from range from 1 Gy to about 50 Gy, and may encompass intermediate ranges including, for example, from 1 to 5, 10, 15, 20, 25, 30, 40, up to 50 Gy in hypofractionated doses. Intensity-modulated radiation therapy (IMRT) may also be used. IMRT is an advanced mode of high-precision three-dimensional conformal radiation therapy (3DCRT), which uses computer-controlled linear accelerators to deliver precise radiation doses to a malignant tumor or specific areas within the tumor. In 3DCRT, the profile of each radiation beam is shaped to fit the profile of the target from a beam's eye view (BEV) using a multileaf collimator (MLC), thereby producing a number of beams. IMRT allows the radiation dose to conform more precisely to the three-dimensional (3-D) shape of the tumor by modulating the intensity of the radiation beam in multiple small volumes. Accordingly, IMRT allows higher radiation doses to be focused to regions within the tumor while minimizing the dose to surrounding normal critical structures. IMRT improves the ability to conform the treatment volume to concave tumor shapes, for example, when the tumor is wrapped around a vulnerable structure, such as the spinal cord or a major organ or blood vessel.

### Subject

The terms “subject” and “patient” can be used interchangeably herein. “Patient in need thereof” or “subject in need thereof” refers to a mammalian subject, preferably human, diagnosed with or suspected of having a disease, whom will be or has been administered an anti-CD38 antibody according to a method of the invention. “Patient in need thereof” or “subject in need thereof” includes those subjects already with the undesired physiological change or disease well as those subjects prone to have the physiological change or disease.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older. In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject has stage I (e.g., ISS stage I) multiple myeloma. In other embodiments, the subject has stage II (e.g., ISS stage II) multiple myeloma. In yet other embodiments, the subject has stage III (e.g., ISS stage III) multiple myeloma.

In some embodiments, the subject has IgG myeloma. In some embodiments, the subject has IgA myeloma. In some embodiments, the subject has light-chain only myeloma.

In some embodiments, the subject has been diagnosed with multiple myeloma for at least about 1 month, e.g., at least about: 2 months, 3 months, 4 months, 5 months, 6 months, 7 months, 8 months, 9 months, 10 months, 11 months, 1 year, 18 months, 2 years, 30 months, 3 years, 4 years, 5 years, 6 years, 7 years, 8 years, 9 years, 10 years, 11 years, 12 years, 13 years, 14 years, 15 years, 16 years, 17 years, 18 years, 19 years or 20 years.

In some embodiments, the subject is refractory to at least one line of therapy. In certain embodiments, the subject has received at least 1 prior line of anti-myeloma therapy, e.g., at least 2, 3, 4, 5, 6, 7 or 8 prior lines of anti-myeloma therapy. In certain embodiments, the subject has received at least two prior lines of anti-myeloma therapy. In particular embodiments, the at least two prior lines of anti-myeloma therapy comprise a hematopoietic stem cell transplantation (HSCT), a proteasome inhibitor (PI), an immunomodulatory drug (IMiD), a maintenance therapy, or a combination thereof. In some embodiments, the prior lines of anti-myeloma therapy comprise a proteasome inhibitor and an immunomodulatory drug.

In some embodiments, the hematopoietic stem cell transplantation is an allogeneic stem cell transplantation (ASCT).

In some embodiments, the proteasome inhibitor is bortezomib, carfilzomib, or ixazomib. In certain embodiments, the proteasome inhibitor is bortezomib.

In particular embodiments, the immunomodulatory drug is Lenalidomide.

In some embodiments, the cancer has a standard risk cytogenetic profile. In some embodiments, the subject has a high risk cytogenetic profile. Cytogenetic abnormalities can be determined based on suitable methods known to those skilled in the art, e.g., fluorescence in situ hybridization or karyotype testing.

In some embodiments, the subject is naïve to anti-CD38 therapy (i.e., the subject has never been administered an anti-CD38 therapy).

### Efficacy

According to embodiments of the disclosure, a variety of factors can be analyzed to determine whether a particular treatment regimen (e.g., pre-dose steroid tapering, post-dose steroid tapering, or both) is an efficacious approach in treating the hematologic malignancy (e.g., RRMM).

In some embodiments, the method results in:
a) ≥50% reduction of serum M-protein and reduction in 24-hour urinary Mprotein by ≥90%;
b) ≥50% reduction of serum M-protein and reduction in 24-hour urinary Mprotein to <200 mg/24 hours;
c) a decrease of ≥50% in the difference between involved and uninvolved FLC levels;
d) ≥50% reduction in bone marrow PCs;
e) ≥50% reduction in the size of soft tissue plasmacytomas, or
a combination thereof.

In some embodiments, the method elicits at least a partial response (PR) in the subject (e.g., according to the International Myeloma Working Group (IMWG) criteria). IMWG criteria for PR: greater than or equal to (>=) 50 percent (%) reduction of serum M-protein and reduction in 24-hour urinary M-protein by >=90% or to less than (<) 200 milligram (mg)/24 hours, If the serum and urine M-proteins are not measurable, a decrease of >=50% in the difference between involved and uninvolved free light chain (FLC) levels were required in place of the M-protein criteria, If serum and urine M-protein are not measurable, and serum free light assay was also not measurable, >=50% reduction in bone marrow plasma cells (PCs) was required in place of M-protein, provided baseline bone marrow plasma cell percentage was >=30%. In addition to the above criteria, if present at baseline, a >=50% reduction in the size of soft tissue plasmacytomas was also required. Also see, e.g., supplementary appendix to Lokhorst H M, et al. N Engl J Med. 2015; 373(13):1207-19.

In some embodiments, the method results in ≥90% reduction in serum M-protein plus urine M-protein <100 mg/24 hours. In certain embodiments, serum and urine M-component are detectable by immunofixation but not on electrophoresis.

In certain embodiment, the method elicits at least a very good partial response (VGPR) in the subject (e.g., according to the IMWG criteria).

In some embodiments, the method results in:
a) negative immunofixation on the serum and urine;
b) disappearance of any soft tissue plasmacytomas;
c) <5% PCs in bone marrow, or
a combination thereof.

In some embodiments, the method results in:
a) negative immunofixation on the serum and urine;
b) disappearance of any soft tissue plasmacytomas; and
c) <5% PCs in bone marrow.

In certain embodiments, the method elicits a complete response (CR) in the subject (e.g., according to the IMWG criteria).

In some embodiments, the method results in:
a) negative immunofixation on the serum and urine;
b) disappearance of any soft tissue plasmacytomas;
c) <5% PCs in bone marrow;
d) normal FLC ratio; and
e) absence of clonal PCs by immunohistochemistry, immunofluorescencea or 2- to 4-color flow cytometry.

In particular embodiments, the method elicits a stringent complete response (sCR) in the subject (e.g., according to the IMWG criteria).

In some embodiments, the method results in stable disease (SD) according to the IMWG criteria.

In some embodiments, the method is used to treat a patient population.

In some embodiments, the patient population achieves an overall response rate (ORR) of at least about 25.0%, e.g., at least about: 30.0%, 35.0%, 40.0%, 45.0%, 50.0% or 55.0%. “ORR” refers to the percentage of subjects who achieve complete response or partial response according to the IMWG criteria, during or after study treatment. In particular embodiments, the ORR is at least about 35.0% or 40.0%. In some embodiments, the ORR is about 25.0-55.0%, e.g., about: 30.0-55.0%, 30.0-50.0%, 35.0-50.0%, 35.0-45.0% or 40.0-45.0%. In particular embodiments, the ORR is at least about 40.0-45.0%. In some embodiments, the ORR is about: 25.0%, 30.0%, 35.0%, 40.0%, 45.0%, 50.0% or 55.0%.

In some embodiments, the patient population achieves a rate of very good partial response (VGPR) or better of at least about 5.0%, during or after treatment, e.g., at least about: 10%, 11.0%, 12.0%, 13.0%, 14.0%, 15.0%, 16.0%, 17.0%, 18.0%, 19.0%, 20.0%, 21.0%, 22.0%, 23.0%, 24.0%, 25.0%, 26.0%, 27.0%, 28.0%, 29.0%, 30%, 31.0%, 32.0%, 33.0%, 34.0%, 35.0%, 36.0%, 37.0%, 38.0%, 39.0% or 40%, during or after treatment. In particular embodiments, the VGPR or better rate is at least about 10%, 15.0%, 20.0%, 25.0% or 30.0%. In some embodiments, the VGPR or better rate is about 5.0-50.0%, e.g., about: 10.0-50.0%, 10.0-45.0%, 15.0-45.0%, 15.0-40.0% 15.0-37.5.0%, 20.0-37.5.0%, 20.0-35.0.0%, 25.0-35.0.0%, 25.0-30.0.0% or 30.0-35.0%.

In some embodiments, the method results in a duration of response (DR) of at least about 9 months, e.g., at least about: 12, 18, 24, 30, 36, 42, 48, 54 or 60 months. “Duration of Response” or “DR” refers to the time from date of initial documentation of response (PR or better) to date of first documented progressive disease (PD), as defined by IMWG criteria. IMWG criteria for PD: Increase of 25% from lowest response value in any one of the following: Serum M component (absolute increase must be >=0.5 grams per deciliter (g/dL), Urine M-component (absolute increase must be >=200 mg/24 hours), Participants without measurable serum and urine Mprotein levels: difference between involved and uninvolved free light chain (FLC) levels (absolute increase must be >10 milligrams per deciliter (mg/dL), participants without measurable serum and urine M-protein levels and without measurable disease by FLC levels, bone marrow PC% (absolute percentage must be >=10%), definite development of new bone lesions or soft tissue plasmacytomas or increase in size of bone lesions or tissue plasmacytomas and development of hypercalcemia (serum calcium >11.5 mg/dL) that can be attributed solely to PC proliferative disorder. Also see, e.g., supplementary appendix to Lokhorst H M, et al. N Engl J Med. 2015; 373(13):1207-19.

In some embodiments, the method results in progression free survival (PFS) of at least about 9 months, e.g., at least about: 12, 18, 24, 30, 36, 42, 48, 54 or 60 months. “progression free survival” or “PFS” refers to the time from date of randomization to either progression of disease (PD), death due to any cause, whichever occurs first.

In some embodiments, the method results in a time to partial response (PR) or better of about 12 months or less, e.g., about: 11, 10, 9, 8, 7, 6, 5, 4, 3, 2 or 1 month or less. “Time to partial response (PR) or better” refers to the time from the date of first dose of study treatment to the date of the first documentation of observed response (CR or PR or better than PR).

In some embodiments, the method improves one or more outcome measurements of the subject. In some embodiments, the one or more outcome measurements comprise progression-free survival, duration of response, or at least partial response, or any combination thereof. In some embodiments, the one or more outcome measures comprise a partial response, a very good partial response, a complete response, or a stringent complete response.

In some embodiments, the subject experiences an improvement in one or more outcome measures consistent with a subject receiving anti-CD38 antibody administration and continuous corticosteroid administration. In other words, the difference in improvement in the subject treated with a method recited herein and a subject treated without a reduction or elimination of corticosteroid administration is not (statistically) significant.

In some embodiments, the subject experiences an increased improvement in one or more outcome measures compared with a subject receiving anti-CD38 antibody administration and continuous corticosteroid administration. In other words, there is an improvement in the subject treated with a method recited herein and a subject treated without a reduction or elimination of corticosteroid administration.

### Safety

According to embodiments of the disclosure, a variety of factors can be analyzed to determine whether a particular treatment regimen (e.g., pre-dose steroid tapering, post-dose steroid tapering, or both) is a safe approach in treating the hematologic malignancy (e.g., RRMM).

As used herein, the term “safe” as it relates to a composition, dose, dosage regimen, treatment or method with a therapeutic or a drug comprising an anti-CD38 antibody (such as daratumumab), refers to a favorable benefit:risk ratio with an acceptable frequency and/or acceptable severity of adverse events (AEs) and/or treatment-emergent adverse events (TEAEs) compared to the standard of care or to another comparator.

“A method of providing safe treatment” or “a method of providing safe administration” refers to a method of administration that is effective to provide the benefits of a therapeutic or pharmaceutical composition, without causing unacceptable adverse events, when administered to a subject.

“Adverse event” or “AE” refers to any untoward medical occurrence in a subject administered an antibody that specifically binds CD38, such as daratumumab. An AE does not necessarily have a causal relationship with the treatment. An AE can therefore be any unfavorable and unintended sign (including an abnormal finding), symptom, or disease temporally associated with the use of a medicinal (investigational or non investigational) product, whether or not related to the antibody that specifically binds CD38, such as daratumumab.

“Treatment emergent adverse events” (TEAE) as used herein takes its customary meaning as will be understood by a person skilled in the art of designing, conducting, or reviewing clinical trials and refers to an AE considered associated with the use of an antibody that specifically binds CD38 if the attribution is possible, probable, or very likely.

In some embodiments, the subject lacks any grade TEAE during or after treatment. Non-limiting examples of any grade TEAE include anemia, arthralgia, asthenia, cough, diarrhea, dizziness, erythema, fatigue, headache, muscle spasms, nasopharyngitis, nausea, peripheral edema, pain in extremity, pyrexia and upper respiratory tract infection.

In some embodiments, the subject lacks grade 3/4 TEAE during or after treatment. Non-limiting examples of grade 3/4 TEAE include anemia, bone pain, lymphopenia and neutropenia.

“Unacceptable adverse events” and “unacceptable adverse reaction” shall mean all harm or undesired outcomes associated with or caused by administration of a pharmaceutical composition or therapeutic, and the harm or undesired outcome reaches such a level of severity that a regulatory agency deems the pharmaceutical composition or therapeutic unacceptable for the proposed use. Examples of unacceptable adverse events or reactions when used in the context of subcutaneous administration of an anti-CD38 antibody include, but are not limited to, thrombocytopenia, neutropenia, severe systemic injection related reactions, and depletion of CD38⁺ cells to below certain specified levels.

Safety of a certain dosage of subcutaneously administered anti-CD38 antibody can be assessed, for example, by immunogenicity studies (e.g., measuring the production of anti-daratumumab antibodies); evaluating changes in CD38 expression levels; assessing the degree and duration of depletion of CD38 expressing cell counts (e.g., plasma cells, natural killer (NK) cells, percent total of lymphocytes); and determining the effects on blood biomarkers, such as serum proteins (e.g., cytokines, chemokines, and inflammatory proteins) by protein profiling. The safety of subcutaneously administered anti-CD38 antibody can also be monitored by physical examination of the subject; observation of local injection site reactions, systemic injection related reactions and other allergic reactions; electrocardiograms; clinical laboratory tests; vital signs; concomitant medications; and monitoring of other adverse events.

### Pharmacokinetics and Immunogenicity

In some embodiments, the method further comprises measuring a production of antibodies specific for the anti-CD38 antibody in the subject after subcutaneous administration of the anti-CD38 antibody.

In some embodiments, the method further comprises measuring a change in CD38 expression level in the subject after subcutaneous administration of the anti-CD38 antibody.

In some embodiments, the method further comprises measuring a degree of depletion of CD38 expressing cells in the subject after subcutaneous administration of the anti-CD38 antibody.

In some embodiments, the method further comprises measuring serum concentrations of the anti-CD38 antibody, e.g., on Cycle 3 Day 1 (pre-dose). In certain embodiments, the serum concentrations of the anti-CD38 antibody (e.g., daratumumab) is between about 500 μg/mL and about 800 μg/mL, for example, about: 525-800, 525-775, 550-775, 550-750, 575-750, 575-725, 600-725 or 600-700 μg/mL. In particular embodiments, the serum concentrations of the anti-CD38 antibody (e.g., daratumumab) is about: 500, 525, 550, 575, 600, 625, 650, 675, 700, 725, 750, 775 or 800 μg/mL.

In some embodiments, the method further comprises measuring a duration of depletion of CD38 expressing cells in the subject after subcutaneous administration of the anti-CD38 antibody. In certain embodiments, the CD38 expressing cells comprise plasma cells, NK cells, lymphocytes, or a combination thereof.

In some embodiments, the method further comprises profiling biomarkers in the subject after subcutaneous administration of the anti-CD38 antibody. In certain embodiments, the biomarkers comprise blood biomarkers. In particular embodiments, the biomarkers comprise serum proteins (e.g., cytokines, chemokines, and inflammatory proteins).

In some embodiments, the method further comprises physically examining the subject after subcutaneous administration of the anti-CD38 antibody.

In some embodiments, the method further comprises detecting an allergic reaction (e.g., a local injection site reaction or a systemic injection related reaction) in the subject.

In some embodiments, the method further comprises performing an electrocardiogram in the subject after subcutaneous administration of the anti-CD38 antibody.

In some embodiments, “safe treatment” and “safe administration” when used with respect to subcutaneous administration of daratumumab, mean reduced adverse events including, but not limited to, reduced depletion of CD38⁺ cells, such as plasma cells, NK cells, T-cells, B-cells, etc., particularly NK cells and/or plasma cells. In a particular embodiment, “safe treatment” and “safe administration” mean that subcutaneous administration of an anti-CD38 antibody (such as daratumumab) results in less than 80% depletion of CD38⁺ cells (e.g., plasma cells, NK cells, T-cells, B-cells, etc.), preferably for at least four (4) weeks after administration of daratumumab. NK cells are a type of lymphocyte (white blood cell) and a component of the innate immune system. NK cells are cytotoxic, and play a role in, e.g., host-rejection of tumors and virally infected cells.

NK cells are a type of cytotoxic lymphocyte important for the innate immune system, are one of the key effector cells for ADCC-mediated depletion of CD38⁺ cells. NK cells are known to express CD38, thus the number of NK cells in circulation may decline following anti-CD38 antibody treatment. Additionally, plasma cells express CD38 and thus will be susceptible to the anti-CD38 antibody mediated cell lysis. Plasma cells are white blood cells that secrete antibody molecules, which recognize and bind foreign substances, and initiate neutralization or destruction of the substance. Depletion of NK cells and plasma cells is measured relative to the amount of NK cells and plasma cells in the subject prior to administration of the anti-CD38 antibody. Any method known in the art in view of the present disclosure can be used to determine the depletion of NK cells and plasma cells, including, but not limited to, flow cytometry.

In some embodiments, the subject has less than about 80% depletion of NK cells about four (4) weeks after subcutaneous administration of the anti-CD38 antibody, e.g., less than about: 70%, 60%, 50%, 40%, 30%, 20% or 10% depletion of NK cells about four (4) weeks after subcutaneous administration of the anti-CD38 antibody.

In some embodiments, the subject has less than about 80% depletion of NK cells about two (2) weeks after subcutaneous administration of the anti-CD38 antibody, e.g., less than about: 70%, 60%, 50%, 40%, 30%, 20% or 10% depletion of NK cells about two (2) weeks after subcutaneous administration of the anti-CD38 antibody.

In some embodiments, the subject has less than about 80% depletion of plasma cells about four (4) weeks after subcutaneous administration of the anti-CD38 antibody, e.g., less than about: 70%, 60%, 50%, 40%, 30%, 20% or 10% depletion of plasma cells about four (4) weeks after subcutaneous administration of the anti-CD38 antibody.

In some embodiments, the subject has less than about 80% depletion of plasma cells about two (2) weeks after subcutaneous administration of the anti-CD38 antibody, e.g., less than about: 70%, 60%, 50%, 40%, 30%, 20% or 10% depletion of plasma cells about two (2) weeks after subcutaneous administration of the anti-CD38 antibody.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

Throughout this specification and the claims which follow, unless the context requires otherwise, the word “comprise,” and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

### Exemplification

Daratumumab is a human IgGκ monoclonal antibody targeting CD38 with a direct on-tumor (de Weers et al., J Immunol 186(3):1840-48 (2011); Lammerts et al., Blood 124(21):3474 (2014); Overdijk et al., J Immunol 197(3):807-13 (2016); Overdijk et al., MAbs 7(2):311-21 (2015)) and immunomodulatory (Adams et al., Cytometry A 95(3):279-89 (2019); Casneuf et al., Leukemia 35:573-84 (2020); Krejcik et al., Blood 128(3):384-94 (2016)) mechanism of action. Daratumumab is approved as monotherapy or in combination with standard-of-care regimens for the treatment of relapsed or refractory multiple myeloma (RRMM). Daratumumab 16 mg/kg is approved for intravenous (IV) infusion as monotherapy or in combination regimens for the treatment of relapsed or refractory multiple myeloma (RRMM) or newly diagnosed multiple myeloma (NDMM) (DARZALEX® (daratumumab) injection, for intravenous use (www_janssenlabels_com/package-insert/product-monograph/prescribing-information/DARZALEX-pi.pdf) (2022)); (DARZALEX FASPRO® (daratumumab and hyaluronidase-fihj) injection, for subcutaneous use (www_janssenlabels_com/package-insert/product-monograph/prescribing-information/DARZALEX+Faspro-pi.pdf) (2022)).

In clinical studies, the median durations of the first, second, and subsequent daratumumab IV infusions were approximately 7, 4, and 3 hours, respectively (DARZALEX® (daratumumab) injection, for intravenous use (www_janssenlabels_com/package-insert/product-monograph/prescribing-information/DARZALEX-pi.pdf) (2022)). In addition, infusion-related reactions (IRRs) have been reported with daratumumab, which occur primarily with the first infusion, are generally mild to moderate in severity, and are manageable (DARZALEX® (daratumumab) injection, for intravenous use (www_janssenlabels_com/package-insert/product-monograph/prescribing-information/DARZALEX-pi.pdf) (2022); Chari et al., Blood 130(8):974-81 (2017); Dimopoulos et al., N Engl J Med 375(14):1319-31 (2016); Lokhorst et al., N Engl J Med 373(13):1207-19 (2015); Lonial et al., Lancet 387(10027):1551-60 (2016); Mateos et al., N Engl J Med 378(6):518-28 (2018); Palumbo et al., N Engl J Med 375(8):754-66 (2016)).

Due to the longer infusion times required for IV administration of daratumumab and the incidence of treatment-related IRRs, a subcutaneous delivery method was developed with the goal to reduce the infusion duration without compromising the safety and efficacy of daratumumab treatment, thereby improving convenience for patients and healthcare providers (Chari et al., Blood 134(5):421-31 (2019); Mateos et al., Lancet Haematol 7(5):e370-e380 (2020); San-Miguel et al., Haematologica 106(6):1725-32 (2021); Usmani et al., Blood 134(8):668-77 (2019)).

The phase 1 PAVO clinical trial was the first study to evaluate the safety, pharmacokinetics, and efficacy of subcutaneous (SC) administration of daratumumab combined with recombinant human hyaluronidase PH2O (rHuPH20; ENHANZE® drug delivery technology, Halozyme, Inc., San Diego, CA) (DARA SC) in patients with RRMM. In Part 1 of PAVO, a mix-and-deliver formulation of daratumumab (20 mg/mL) and rHuPH20 (DARA MD) given by means of a syringe pump at doses of 1,2000 mg and 1,800 mg over 20-30 minutes showed that DARA SC administration was feasible in patients with RRMM. The safety, pharmacokinetics (PK), immunogenicity, and efficacy results with the DARA MD 1,800 mg dose were consistent with those associated with daratumumab IV and induced deep and durable responses (Usmani et al., Blood 134(8):668-77 (2019)). In Part 2 of the study, a concentrated, pre-mixed SC formulation of daratumumab (DARA SC; 1,800 mg daratumumab co-formulated with 30,000 U rHuPH20 (2,000 U/mL, 15 mL)) was administered to patients by manual subcutaneous injection into the abdomen over 3 to 5 minutes. The tolerability profile of DARA SC was consistent with that of daratumumab IV, with no new safety concerns observed. DARA SC reduced the administration time and demonstrated low IRR rates in patients with RRMM. DARA SC achieved maximum C_{trough} values that were similar to or greater than those reached with daratumumab IV, with a reduced administration time and no new safety concerns (San-Miguel et al., Haematologica 106(6):1725-32 (2020)). Importantly, the efficacy of DARA SC was similar to what was previously observed with daratumumab IV (Usmani et al., Blood 128(1):37-44 (2016)). After 14.2 months of follow-up in PAVO Part 2, the overall response rate (ORR) was 52%, and the median duration of response was 15.7 months (San-Miguel et al., Haematologica 106(6):1725-32 (2020)). After 7.5 months of follow-up in the phase 3 COLUMBA study, the overall response rate was 41% with DARA SC and 37% with DARA IV; the median duration of response was not reached for either group (Mateos et al., Lancet Haematol 7(5):e370-e380 (2020)). Based on the efficacy and safety data of DARA SC in patients with multiple myeloma (Mateos et al., Lancet Haematol 7(5):e370-e380 (2020)), DARA SC received approval in the United States, European Union, and other countries globally as monotherapy for RRMM and in combination regimens for the treatment of RRMM or NDMM (DARZALEX FASPRO® (daratumumab and hyaluronidase-fihj) injection, for subcutaneous use (www_janssenlabels_com/package-insert/product-monograph/prescribing-information/DARZALEX+Faspro-pi.pdf) (2022).

Corticosteroids serve as an important component of the treatment regimens for patients with multiple myeloma. However, long-term use of corticosteroids may lead to additive toxicities to treatment regimens, which are now including up to 4 individual drugs, and may subsequently negatively affect the quality of life for patients with multiple myeloma. Indeed, patients have indicated a preference for treatment regimens that include limited steroid use (Parsons et al., BMC Cancer 19(1):264 (2019)). In addition, the immunosuppressive effect of corticosteroids may reduce the efficacy of immunotherapies for cancer treatment such as checkpoint inhibitors, T-cell redirectors, or chimeric antigen receptor (CAR) T cell therapy (Arbour et al., J Clin Oncol 36(28):2872-78 (2018); Namuduri et al., Expert Rev Hematol. 9(6):511-13 (2016); Strati P, et al., Blood 137(23):3272-76 (2021); Kauer J, et al., J Immunother Cancer 8(1):e000621 (2020)). Therefore, while corticosteroids may be continued as part of combination therapy in patients with cancer, corticosteroid tapering, particularly with DARA SC, may achieve consistent tolerability without a loss of efficacy. Part 3 of the PAVO study was conducted to evaluate the safety of different schedules of pre- and post-dose corticosteroid tapering during DARA SC administration.

### EXAMPLE 1

### Methods

Study Design and Patient Population

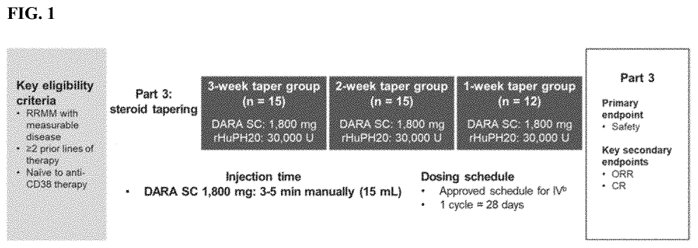

The PAVO (MMY1004) trial is an open-label, nonrandomized, multicenter, phase lb study consisting of 3 parts. Detailed eligibility criteria were previously published (San-Miguel et al., Haematologica 106(6):1725-32 (2021); Usmani et al., Blood 134(8):668-77 (2019)). Briefly, eligible patients aged ≥18 years had measurable RRMM, ≥2 prior lines of treatment, including a proteasome inhibitor (PI) and immunomodulatory drug (IMiD), Eastern Cooperative Oncology Group Performance Status (ECOG PS) score ≤2, and no previous treatment with daratumumab or other anti-CD38 therapies (FIG. 1). Safety was assessed, with an emphasis on IRRs, while corticosteroids were reduced and discontinued.

Treatment

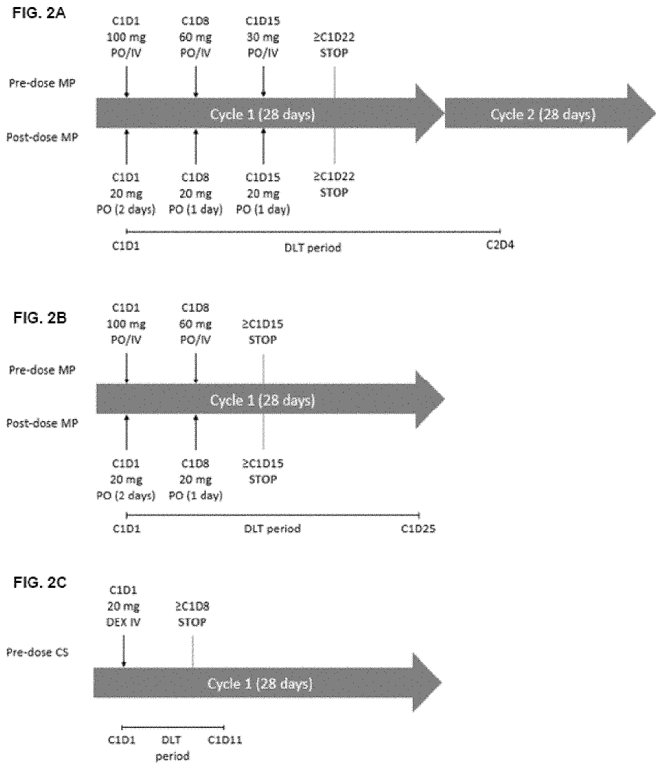

The purpose of Part 3 was to evaluate the safety of daratumumab 1,800 mg SC delivery without predose and postdose corticosteroids after a 3-week, 2-week, and 1-week tapering schedule to assess the safety of corticosteroid-free SC daratumumab administration. A total of 42 participants were treated. In Part 3, patients received DARA SC (DARA 1,800 mg+rHuPH20 30,000 U in 15 mL) by manual SC injection (over 3-5 minutes; alternating locations on the abdomen) per an approved IV monotherapy dosing schedule (FIG. 1): once weekly during Cycles 1 and 2; every 2 weeks during Cycles 3-6; and every 4 weeks thereafter. In conjunction, patients also received a 3-week, 2-week, or 1-week steroid tapering schedule (FIG. 2). The 3-week tapering schedule (corticosteroid-free by Cycle 1 Day 22) consisted of methylprednisolone (MP) given orally (PO)/IV pre-dose (Cycle 1 Day 1, 100 mg; Cycle 1 Day 8, 60 mg; Cycle 1 Day 15, 30 mg) and PO post-dose (Cycle 1 Day 1, 20 mg for 2 days; Cycle 1 Day 8, 20 mg for 1 day; Cycle 1 Day 15, 20 mg for 1 day). The 2-week tapering schedule (corticosteroid-free by Cycle 1 Day 15) consisted of MP given PO/IV pre-dose (Cycle 1 Day 1, 100 mg; Cycle 1 Day 8, 60 mg) and PO post-dose (Cycle 1 Day 1, 20 mg for 2 days; Cycle 1 Day 8, 20 mg for 1 day). The 1-week tapering schedule (corticosteroid-free by Cycle 1 Day 8) consisted of dexamethasone 20 mg administered IV pre-dose on Cycle 1 Day 1, without post-dose administration.

The 3-week, 2-week and 1-week steroid tapering schedules were assessed by “3+3” design, followed by cohort expansion to approximately 15 patients (3-week and 2-week tapering cohorts) or 12 patients (1-week tapering cohort). The dose-limiting toxicity (DLT) assessment period in the 3-week, 2-week and 1-week tapering cohorts were Cycle 1 Day 1 through Cycle 2 Day 4, Cycle 1 Day 1 through Cycle 1 Day 25, and Cycle 1 Day 1 through Cycle 1 Day 11, respectively. A DLT was defined as a grade 4 IRR within 72 hours of injection or a grade 3 IRR within 72 hours of injection that did not resolve with slowing or stopping the injection and included supportive care and symptomatic therapy. Grade 3 or 4 IRRs only qualified as DLTs if they occurred during corticosteroid reduction or discontinuation (not during Cycle 1 Day 1). For the 3-week and 2-week groups, IRRs reported at Cycle 1 Day 1 were not considered DLTs, as corticosteroids had not yet been reduced or discontinued. Up to approximately 12 patients could be enrolled in the 1-week tapering schedule to assess safety, including IRRs.

Endpoints and Assessments

The primary endpoint was safety of pre- and post-dose steroid tapering. Key secondary endpoints included the overall response rate (ORR) and the rate of complete response (CR).

Safety assessments included treatment-emergent adverse events (TEAEs), serious adverse events, and IRRs. All toxicities were graded according to the National Cancer Institute's Common Terminology for the Classification of Adverse Events (NCI-CTCAE) Version 4.03 (National Cancer Institute. Common Terminology Criteria for Adverse Events (CTCAE): Version 4.03.).

For pharmacokinetic analyses, DARA SC serum concentrations were evaluated from blood samples drawn pre-dose on Cycle 3 Day 1. For evaluation of immunogenicity, anti-daratumumab antibodies in serum and anti-rHuPH20 antibodies in plasma were assessed pre-dose on Cycle 1 Day 1, Cycle 1 Day 15, Cycle 2 Day 22, Cycle 4 Day 1 and 4- and 8-weeks post-treatment. The presence of anti-daratumumab and/or anti-rHuPH20 antibodies was assessed for classification as neutralizing antibodies.

For the evaluation of efficacy, responses were assessed according to International Myeloma Working Group (IMWG) consensus recommendations (Durie et al., Leukemia 20(9):1467-73 (2006); Rajkumar et al., Blood 117(18):4691-95 (2011)). Disease evaluations were performed by a central laboratory.

Statistical Analyses

In PAVO Part 3, no formal statistical hypothesis testing was performed. Data were summarized descriptively. Continuous variables were summarized using the number of observations, mean and standard deviation (SD), coefficient of variation, median and range as appropriate. Categorical variables were summarized using the number of observations and percentages as appropriate. Duration of response and PFS were estimated using Kaplan-Meier methods.

The safety and efficacy populations, defined as all patients who received ≥1 dose of study drug, was the primary population for analysis. The pharmacokinetics analysis set included all subjects who received at least 1 administration of study drug and had at least 1 pharmacokinetics sample concentration value after the first drug administration. The pharmacokinetic-evaluable population was defined as all patients who received ≥1 dose of study drug and provided ≥1 post-infusion pharmacokinetic sample. The immunogenicity population was defined as all patients who received ≥1 dose of study drug and provided ≥1 post-infusion immunogenicity sample.

### EXAMPLE 2

### Patient Disposition and Demographics

A total of 42 patients were enrolled in PAVO Part 3 (3-week tapering cohort, n=15; 2-week tapering cohort, n=15; 1-week tapering cohort, n=12). Overall, the median age was 69.5 years (range: 52-86), and median weight was 77.8 kg (range: 44.0-151.3). At baseline, 92.9% (39/42) of patients had an ECOG PS score <1. Median time from diagnosis was 5.9 years (range: 0.7-19.2) and the median number of prior lines of therapy was 3 (range: 2-7). A total of 19 (45.2%) patients were refractory to a PI and an IMiD. Of the 31 patients with available cytogenetic data, 8 (25.8%) patients had high cytogenetic risk abnormalities (Table 2).

Median (range) duration of follow-up was 8.3 months for the all-treated population: 9.2 (1.9-25.5) months for the 3-week tapering cohort, 11.1 (1.7-24.0) months for the 2-week tapering cohort, and 8.3 (0.4-13.1) months for the 1-week tapering cohort. The all-treated population included all patients who received ≥1 dose of study drug. The median treatment duration was 6.5 months in all steroid tapering groups. Patients in the 3-week tapering cohort received a median (range) of 18 (5-38) DARA SC doses. Patients in the 2-week tapering cohort received a median (range) of 17 (5-33) DARA SC doses. Patients in the 1-week tapering cohort received a median (range) of 18 (2-25) DARA SC doses. Thirteen (86.7%) patients each in the 3-week and 2-week tapering cohorts, and 7 (58.3%) patients in the 1-week tapering cohort discontinued treatment; most discontinuations were due to progressive disease (10 [66.7%], 12 [80.0%], and 5 [41.7%] patients, respectively). At the time of the analysis a total of 2 (13.3%), 2 (13.3%), and 5 (41.7%) patients in the 3-week, 2-week, and 1-week tapering cohorts, respectively, were still receiving treatment.

### EXAMPLE 3

### Safety, Pharmacokinetics, Immunogenicity and Efficacy

Treatment-Emergent Adverse Events

All patients, regardless of steroid-tapering cohort, experienced ≥1 TEAE (Tables 3-5). A total of 21 (50.0%) patients reported a grade >3 TEAE, a total of 18 (42.9%) patients reported a grade 3/4 TEAE, and 16 (38.1%) patients experienced a serious TEAE. In the 3-week tapering cohort, the most common any grade TEAE was nausea, which occurred in 8 (53.5%) patients, and the most common grade ≥3 TEAE was lymphopenia, which occurred in 2 (13.3%) patients (Table 3). In the 2-week tapering cohort, the most common TEAE was nasopharyngitis (5 [33.3%] patients) and the most common grade ≥3 TEAE was neutropenia (3 [20.0%] patients). In the 1-week tapering cohort, the most common TEAEs were anemia, diarrhea, asthenia, and peripheral edema (4 [33.3%] patients each) and the most common grade ≥3 TEAE was anemia, which occurred in 2 (16.7%) patients. A total of 6 patients died during the study. Grade 5 TEAEs occurred in 2 (16.7%) patients in the 1-week tapering cohort (staphylococcal pneumonia and pulmonary embolism) and occurred in 1 (6.7%) patient in the 2-week tapering group (general physical health deterioration). One patient in the 3-week tapering cohort died due to complications from diffuse large B-cell lymphoma (DLBCL). In addition, 3 patients (1 in each cohort) died due to progressive disease during the course of the study. TEAEs with an outcome of death were reported in 2 participants in the 1-week tapering cohort (pulmonary embolism and pneumonia staphylococcal); neither of the events were treatment-related. No deaths were reported in the 2-week and 3-week tapering cohorts.

TEAEs in the System Organ Class (SOC) of General disorders and administration site conditions were the most common for all three parts (71.1% for Part 1, 72.0% for Part 2, and 71.4% for Part 3) followed by the SOC of Infections and infestations (71.1%, 72.0%, and 64.3%, respectively). The most frequently reported serious TEAEs were in the SOC of Infections and infestations (17.8% in Part 1, 12.0% in Part 2, and 11.9% in Part 3), and General disorders and administration site conditions (6.7%, 8.0%, and 4.8%, respectively). The most frequently reported TEAEs of Grade >3 were lymphopenia (20.0% in Part 2), and anemia (15.6% in Part 1, and 9.5% in Part 3).

No new death occurrence within 30 days of last dose of study treatment was reported since the clinical cutoff (CCO) for the previous Part 1, Part 2, and Part 3 analyses. No deaths were COVID-19 related.

No additional IRRs have been reported since the CCOs of the previous analyses.

Nausea was more frequent in the 3-week tapering cohort (53% of participants), compared to 20% and 16.7% in the 2-week and 1-week tapering cohorts, respectively. Serious TEAEs occurred in 6 (40.0%) patients in the 3-week group, 6 (40.0%) patients in the 2-week group, and 4 (33.3%) patients in the 1-week group. Nervous system disorders were more frequent in the 3-week cohort (46.7%) compared with the 2-week cohort (26.7%) and 1-week cohort (16.7%). Further details on the most common TEAEs (>10%) are provided in Tables 3 and 4.

Grade ≥3 TEAEs were reported for 60% of participants in the 3-week tapering cohort, 53.3% of participants in the 2-week tapering cohort, and 33.3% of participants in the 1-week tapering cohort. In the 3-week tapering cohort, the most common any grade TEAE was nausea, which occurred in 8 (53.3%) patients, and the most common Grade ≥3 (Grade 3/4) TEAE was lymphopenia, which occurred in 2 (13.3%) patients (Tables 3 and 4). In the 2-week group, the most common TEAE was nasopharyngitis (5 [33.3%] patients) and the most common Grade ≥3 (Grade 3/4) TEAE was neutropenia (3 [20.0%] patients). In the 1-week group, the most common TEAEs were anemia, diarrhea, asthenia, and peripheral edema (4 [33.3%] patients each) and the most common Grade ≥3 (Grade 3/4) TEAE was anemia, which occurred in 2 (16.7%) patients. Refer to Tables 3-5 for more detail.

Infusion-Related Reactions

A total of 5 (11.9%) patients experienced an IRR, all of which occurred with the first administration of DARA SC. In the 3-week, 2-week, and 1-week tapering cohorts, IRRs were reported by 0 (0%), 3 (20.0%), and 2 (16.7%) patients, respectively. The most common IRRs (occurring in ≥5% of patients) were chills and pyrexia (3 [7.1%] patients each). IRRs of tachycardia, increased blood pressure, and oropharyngeal pain were reported by 1 (2.4%) patient each. All IRRs occurred on the first DARA SC administration (Cycle 1 Day 1 dosing), with a median onset time of 79 (range, 31-555) minutes, and resolved the same day, with none occurring after steroid tapering. IRRs were generally mild, with only 1 grade 3 IRR (increased blood pressure; 2-week tapering cohort, resolved on day of onset) and none qualified for dose-limiting toxicity (no grade 4 IRRs reported). None of the IRRs met the definition of a DLT or led to interruption or discontinuation of treatment among patients.

Pharmacokinetics

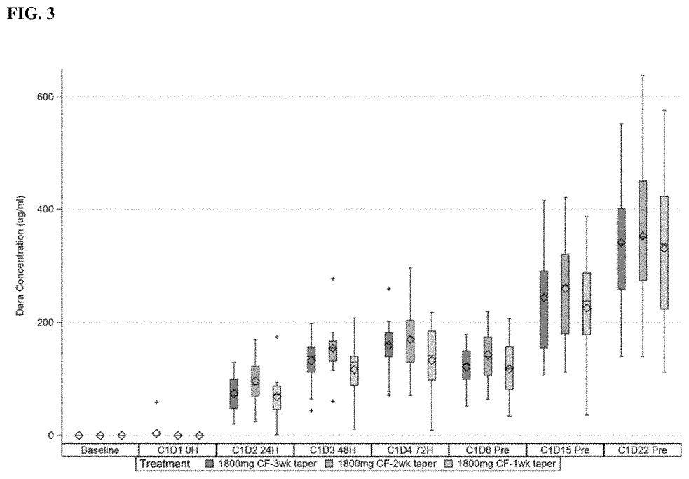

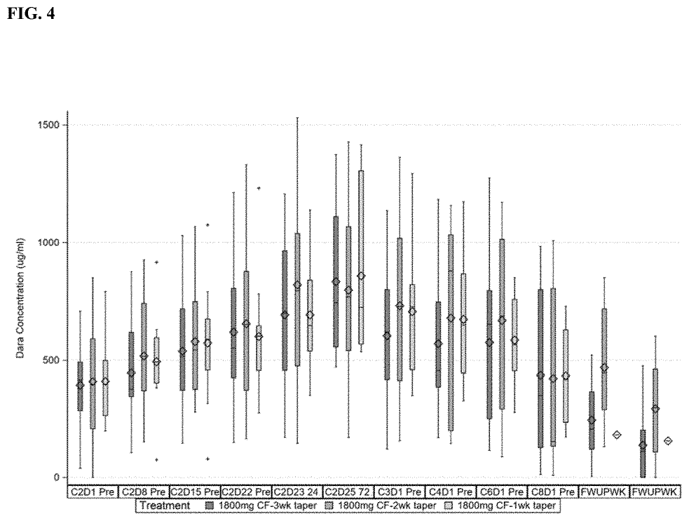

In the total pharmacokinetic-evaluable population (n=37), the mean (standard deviation (SD)) daratumumab serum concentration was 676 (314) μg/mL at Cycle 3 Day 1. In the 3-week, 2-week, and 1-week tapering cohorts, the mean (SD) serum concentration of daratumumab at Cycle 3 Day 1 was 604 (280), 731 (382), and 706 (270) μg/mL, respectively, following weekly dosing of DARA SC. Pharmacokinetic results following administration of 1,800 mg DARA SC were similar with 3-week, 2-week, and 1-week steroid tapering and were consistent with previous reports of DARA SC (Mateos et al., Lancet Haematol. 7(5):e370-e380 (2020)). Changes in serum concentrations of daratumumab during Cycle 1 are shown in FIG. 3, and changes in serum concentrations of daratumumab from Cycle 2 onwards are shown in FIG. 4.

Immunogenicity

Among patients in the daratumumab immunogenicity-evaluable population (n=41), no patient tested positive for the presence of anti-daratumumab antibodies. Among rHuPH20 immunogenicity evaluable patients in the 3-week, 2-week, and 1-week tapering cohorts, 6 (40.0%), 3 (20.0%), and 1 (9.1%) patients tested positive for treatment-emergent anti-rHuPH20 antibodies. None of the anti-rHuPH20 antibodies were neutralizing antibodies or correlated with injection-site reactions.

Efficacy

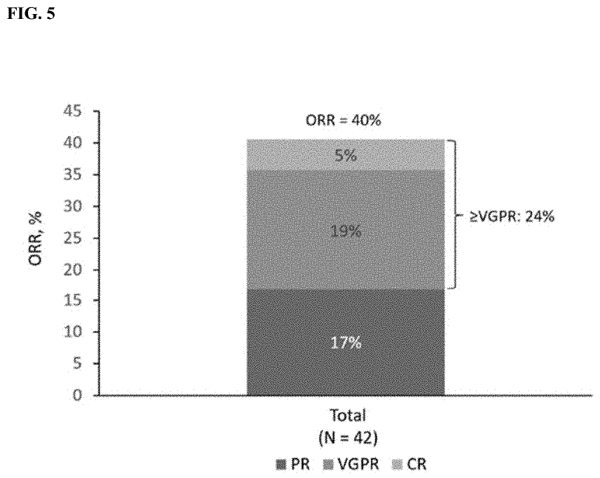

In the total all-treated population (n=42), with a median follow-up of 8.3 months, the ORR was 40.5% (95% CI: 25.6%, 56.7%), with 10 (23.8% (95% CI: 12.1%, 39.5%)) patients achieving ≥VGPR and 2 (4.8% (95% CI: 0.6%, 16.2%)) patients achieving ≥CR among all treated patients (FIG. 5). In the 3-week tapering cohort, the ORR was 40.0%, with 3 (20.0%) patients achieving ≥VGPR and 1 (6.7%) patient achieving ≥CR. In the 2-week tapering cohort, the ORR was 40.0%, with 5 (33.3%) patients achieving ≥VGPR and no patient achieving ≥CR. In the 1-week tapering cohort, the ORR was 41.7%, with 2 (16.7%) patients achieving ≥VGPR and 1 (8.3%) patient achieving ≥CR.

Among responders in the response-evaluable population (n=17), the median time to first response and best response were 1.0 months and 1.1 months, respectively. In the 3-week, 2-week, and 1-week tapering cohorts, median time to best response was 1.5, 1.9, and 1.0 months, respectively. Median duration of response was 16.7 months in the 2-week tapering cohort and was not reached in the 3-week and 1-week tapering cohorts at the time of clinical cut-off; 9-month duration of response rates were 83.3%, 83.3%, and 100% in the 3-week, 2-week, and 1-week tapering cohorts, respectively.

At the clinical cut-off, median PFS was 5.9 months, with an estimated 9-month PFS rate of 40.7% for all treated patients. At a median follow-up of 9.2, 11.1, and 8.3 months for the 3-week, 2-week, and 1-week tapering cohorts, the median PFS was 5.9, 4.7, and 7.4 months in the 3-week, 2-week, and 1-week tapering cohorts, respectively. Estimated 9-month PFS rates were 40.0%, 36.1%, and 46.7% for the 3-week, 2-week, and 1-week tapering cohorts, respectively.

The response rates (FIG. 5) were consistent with response rates observed at the primary analysis of the phase 3 COLUMBA trial (Mateos et al., Lancet Haematol. 7(5):e370-e380 (2020)). The overall response rate was 40.0% (95% CI, 16.3%-67.7%) for both the 3-week and 2-week groups and was 41.7% (95% CI, 15.2%-72.3%) for the 1-week group. Rates of very good partial response or better were 20.0% (95% CI, 4.3%-48.1%) for the 3-week group, 33.3% (95% CI, 11.8%-61.6%) for the 2-week group, and 16.7% (95% CI, 2.1%-48.4%) for the 1-week group, respectively. Among responders, the median duration of response was 16.7 months in the 2-week group; the median duration of response was not reached in either the 3-week or 1-week groups. For all treated patients in Part 3 (N=42), the median progression-free survival (PFS) was 5.9 months with an estimated 9-month PFS rate of 40.7%. At the primary analysis of COLUMBA, the median PFS was 5.6 months for DARA SC (Mateos et al., Lancet Haematol. 7(5):e370-e380 (2020)).

The results suggest that rapid corticosteroid tapering is tolerable in patients with RRMM receiving DARA SC and does not diminish the efficacy of DARA SC in these patients. These data will help guide treatment with future DARA SC combinations where limiting concurrent corticosteroids may be preferred (e.g., T-cell redirectors, CAR-T, or checkpoint inhibitors).

This study in 42 patients with RRMM receiving DARA SC showed that rapid corticosteroid tapering over 1 to 3 weeks was tolerable in patients with RRMM receiving DARA SC, with PK, immunogenicity, and safety results consistent with previous reports of DARA SC. Patients receiving DARA SC with corticosteroid tapering demonstrated similar efficacy to patients receiving DARA IV (Lokhorst H M, et al. N Engl J Med. 2015; 373(13):1207-19 and Lonial et al., Lancet 387(10027):1551-60 (2016)).

Corticosteroids, including dexamethasone and prednisone, have been widely used in the treatment of multiple myeloma for more than 50 years. However, despite their potent activity in multiple myeloma, it is well established that the long-term use of corticosteroids may lead to cumulative toxicities and other clinical sequelae (Burwick et al., Ann Hematol 98(1):19-28 (2019)). Therefore, the clinical benefit of corticosteroid use in multiple myeloma, must be evaluated against and in balance with the potential risk to patients in terms of toxicity and quality of life. Indeed, in a Canadian study evaluating treatment preferences among patients with RRMM, patients prioritized treatments that increased life expectancy as well as those that limited corticosteroid use, due to the negative effects of corticosteroid use including insomnia, cognitive impairment, and mood disturbances (Parsons et al., BMC Cancer 19(1):264 (2019)). Therefore, it is critical to identify effective treatment regimens that limit the use of corticosteroids for patients who experience associated toxicities.

The pharmacokinetic and immunogenicity results were consistent with those previously reported in Part 1 and Part 2 of the study. Subcutaneous administration of DARA SC with 3-week, 2-week, and 1-week corticosteroid tapering schedules appeared to result in similar mean serum daratumumab concentrations. In all three cohorts in Part 3, no patients evaluable for daratumumab immunogenicity were positive for anti-daratumumab antibodies, indicating a low risk for immunogenicity when daratumumab is administered subcutaneously.

Daratumumab-based regimens have consistently demonstrated efficacy in patients with NDMM and RRMM. However, daratumumab treatment is administered in conjunction with corticosteroids including dexamethasone and prednisone for mitigation of IRRs. Part 3 of the PAVO study aimed to determine if tapering off corticosteroids, with DARA SC treatment, maintains adequate tolerability in the context of the occurrence of IRRs without a loss of efficacy. The findings presented here demonstrate that tapering off corticosteroids is safe in patients with RRMM receiving DARA SC. Specifically, tolerability profiles in the 3 cohorts with different schedules of corticosteroid tapering were comparable with previous reports of daratumumab with no increase in IRR rates (Mateos et al., Lancet Haematol. 7(5):e370-e380 (2020); San-Miguel et al., Haematologica 106(6):1725-32 (2021); Usmani et al., Blood 134(8):668-77 (2019)).

Importantly, these results suggest that tapering off corticosteroids does not diminish the efficacy of DARA SC, as patients who received DARA SC while tapering off corticosteroids achieved similar efficacy in terms of response rates and duration of responses compared with patients who received daratumumab SC monotherapy in the presence of corticosteroids (Mateos et al., Lancet Haematol. 7(5):e370-e380 (2020); San-Miguel et al., Haematologica 106(6):1725-32 (2021); Usmani et al., Blood 134(8):668-77 (2019)). In the 3 cohorts combined, the ORR was 40.5% (95% CI: 25.6%, 56.7%), with 10 (23.8% (95% CI: 12.1%, 39.5%)) patients achieving ≥VGPR. For comparison, the ORR observed with DARA MD 1,800 mg in PAVO Part 1 (median follow-up of 8.3 months) was 42.2% (Usmani et al., Blood 134(8):668-77 (2019)). In PAVO Part 2 (median follow-up, 14.2 months), the ORR with DARA SC was 52% (San-Miguel et al., Haematologica 106(6):1725-32 (2021)). In the COLUMBA study, with a median follow-up of 7.5 months, the ORR was 41% with DARA SC and 37% with DARA IV (Mateos et al., Lancet Haematol. 7(5):e370-e380 (2020)).

The newer immunotherapeutic treatment strategies including bispecific T-cell redirecting antibodies and chimeric antigen receptor (CAR) T-cell therapy have demonstrated efficacy in patients with RRMM (Zhou et al., Front Immunol. 11:620312 (2020); Usmani et al., Lancet 398(10301):665-74 (2021); Verkleij et al., Blood Adv. 5(8):2196-2215 (2021)). However, in patients who experience toxicities to treatment including cytokine release syndrome, corticosteroids may be administered to help manage the adverse effects. Unfortunately, corticosteroids can interfere with the anti-tumor effects of novel immunotherapeutic drugs. For example, corticosteroids are contraindicated with CAR T-cell infusion as they may suppress the activity of CAR T cells (Zhou et al., Front Immunol. 11:620312 (2020); Yakoub-Agha et al., Haematologica 105(2):297-316 (2020)). Furthermore, a retrospective study demonstrated that the use of a higher cumulative dose of corticosteroids was associated with shorter PFS and OS outcomes in patients with relapsed or refractory large B-cell lymphoma treated with CAR T-cell therapy (Strati et al., Blood. 137(23):3272-76 (2021)). Additionally, a separate retrospective study demonstrated that baseline corticosteroid use was associated with poorer outcomes in patients with non-small cell lung cancer who were treated with PDL1 blockade (Arbour et al., J Clin Oncol 36(28):2872-78 (2018)). While additional studies are warranted, the findings presented here will help guide the future use of DARA SC combination regimens, including CAR T-cell therapy, bispecific antibodies, and checkpoint inhibitors, where no or limited concurrent corticosteroids may be preferred.

This study in 42 patients with RRMM receiving DARA SC showed that rapid corticosteroid tapering over 1 to 3 weeks was safe, without an increased risk of IRRs and with pharmacokinetic, immunogenicity, and safety results consistent with previous reports of DARA SC. In conclusion, these findings demonstrate that tapering off corticosteroids over 1 to 3 weeks is safe and does not compromise efficacy in patients with relapsed/refractory multiple myeloma who are receiving DARA SC.

Daratumumab is approved for the treatment of patients with multiple myeloma (MM). The safety, pharmacokinetics, and efficacy from Part 3 of the phase 1b PAVO study investigating 3 pre- and post-dose corticosteroid tapering schedules during subcutaneous daratumumab (DARA SC) therapy is reported. MM patients with ≥2 prior treatment lines received DARA SC (daratumumab 1,800 mg+rHuPH20 30,000 U in 15 mL) QW in Cycles 1-2, Q2W in Cycles 3-6, and Q4W thereafter. Patients also received a 3-week tapering schedule (corticosteroid-free by Cycle 1 Day 22) with methylprednisolone (PO/IV pre-dose; PO post-dose), 2-week tapering schedule (corticosteroid-free by Cycle 1 Day 15) with methylprednisolone (PO/IV pre-dose; PO post-dose), or 1-week tapering schedule (corticosteroid-free by Cycle 1 Day 8) with dexamethasone (IV pre-dose). The primary endpoint was safety. Patients (3-week: n=15; 2-week: n=15; 1-week: n=12) received a median of 3 prior treatment lines. No new safety concerns or increased IRR rates were observed with rapid corticosteroid tapering. IRRs were reported in 5 (11.9%) patients, which were generally mild and occurred with the first DARA SC administration. No IRRs occurred with subsequent DARA SC administrations. Mean serum DARA concentrations (m/mL) at Cycle 3 Day 1 (pre-dose) were 604, 731, and 706 μg/mL in the 3-week, 2-week, and 1-week groups, respectively. In total, with a median follow-up of 8.3 months, the overall response rate was 40.5%. At a median 9.2-, 11.1-, and 8.3-months follow-up in the 3-week, 2-week, and 1-week groups, respectively, overall response rates were 40.0%, 40.0%, 41.7%. Rapid corticosteroid tapering over 3 weeks is safe in relapsed/refractory MM patients receiving DARA SC. These data will help guide future DARA SC treatment regimens, where limiting concurrent corticosteroids is preferred.

TABLE 2
Baseline Demographics and Disease Characteristics.

| | DARA SC 1,800 mg | | | |
|---|---|---|---|---|
| | 3-week group | 2-week group | 1-week group | Total |
| | (n = 15) | (n = 15) | (n = 12) | (N = 42) |
| Median age (range), years | 66.0 (59-81) | 69.0 (52-86) | 72.5 (58-84) | 69.5 (52-86) |
| Age category, n (%) | | | | |
| 18-<65 y | 4 (26.7) | 6 (40.0) | 2 (16.7) | 12 (28.6) |
| 65-<75 y | 9 (60.0) | 7 (46.7) | 5 (41.7) | 21 (50.0) |
| ≥75 y | 2 (13.3) | 2 (13.3) | 5 (41.7) | 9 (21.4) |
| Median (range) weight, kg | 77.0 (56.0-151.3) | 81.0 (50.0-100.0) | 76.1 (44.0-103.0) | 77.8 (44.0-151.3) |
| ECOG PS score, n (%) | | | | |
| 0 | 5 (33.3) | 8 (53.3) | 4 (33.3) | 17 (40.5) |
| 1 | 9 (60.0) | 7 (46.7) | 6 (50.0) | 22 (52.4) |
| 2 | 1 (6.7) | 0 | 2 (16.7) | 3 (7.1) |
| ISS disease stage,^{a} n (%) | | | | |
| I | 9 (60.0) | 8 (53.3) | 5 (41.7) | 22 (52.4) |
| II | 4 (26.7) | 2 (13.3) | 6 (50.0) | 12 (28.6) |
| III | 2 (13.3) | 5 (33.3) | 1 (8.3) | 8 (19.0) |
| Type of multiple myeloma,^{b} n (%) | | | | |
| IgG | 9 (60.0) | 8 (53.3) | 7 (58.3) | 24 (57.1) |
| IgA | 1 (6.7) | 3 (20.0) | 3 (25.0) | 7 (16.7) |
| Light chain | 5 (33.3) | 4 (26.7) | 2 (16.7) | 11 (26.2) |
| Median (range) time from | 6.3 (2.3-19.2) | 5.6 (0.7-14.3) | 5.8 (2.0-17.2) | 5.9 (0.7-19.2) |
| diagnosis, y | | | | |
| Median (range) previous lines of | 2 (2-7) | 2 (2-4) | 4 (2-6) | 3 (2-7) |
| therapy | | | | |
| Prior lines of therapy, n (%) | | | | |
| ≤3 | 11 (73.3) | 14 (93.3) | 6 (50.0) | 31 (73.8) |
| >3 | 4 (26.7) | 1 (6.7) | 6 (50.0) | 11 (26.2) |
| Prior ASCT, n (%) | 14 (93.3) | 12 (80.0) | 3 (25.0) | 29 (69.0) |
| Prior PI, n (%) | | | | |
| Bortezomib | 15 (100.0) | 15 (100.0) | 12 (100.0) | 42 (100.0) |
| Prior IMiD, n (%) | | | | |
| Lenalidomide | 15 (100.0) | 14 (93.3) | 10 (83.3) | 39 (92.9) |
| Refractory to, n (%) | | | | |
| Bortezomib | 6 (40.0) | 4 (26.7) | 6 (50.0) | 16 (38.1) |
| Lenalidomide | 7 (46.7) | 8 (53.3) | 9 (75.0) | 24 (57.1) |
| PI and IMiD | 4 (26.7) | 7 (46.7) | 8 (66.7) | 19 (45.2) |
| Last line of therapy | 6 (40.0) | 10 (66.7) | 9 (75.0) | 25 (59.5) |
| Cytogenetic profile,^{c} n (%) | n = 12 | n = 12 | n = 7 | n = 31 |
| Standard risk | 9 (75.0) | 9 (75.0) | 5 (71.4) | 23 (74.2) |
| High risk | 3 (25.0) | 3 (25.0) | 2 (28.6) | 8 (25.8) |

ECOG PS, Eastern Cooperative Oncology Group performance status; ISS, International Staging System; ASCT, autologous stem cell transplant; PI, proteasome inhibitor; IMiD, immunomodulatory drug.
^{a}ISS staging is derived based on the combination of serum β2-microglobulin and albumin.
^{b}By immunofixation.
^{c}Cytogenetic abnormalities are based on fluorescence in situ hybridization or karyotype testing.
Percentages were calculated with the number of patients in each treatment group as the denominator.

TABLE 3
Most Common TEAEs by Preferred Term

| | 3-week group | 2-week group | 1-week group | Total |
|---|---|---|---|---|
| | (n = 15) | (n = 15) | (n = 12) | (n = 42) |
| Any TEAE, n (%) | 15 (100) | 15 (100) | 12 (100) | 42 (100) |
| Any grade ≥3 TEAE, n (%) | 9 (60.0) | 8 (53.3) | 4 (33.3) | 21 (50.0) |
| Most common (≥25%) TEAEs (any | | | | |
| grade), n (%) | | | | |
| Hematologic | | | | |
| Anemia | 1 (6.7) | 2 (13.3) | 4 (33.3) | 7 (16.7) |
| Nonhematologic | | | | |
| Nausea | 8 (53.3) | 3 (20.0) | 2 (16.7) | 13 (31.0) |
| Upper respiratory tract infection | 6 (40.0) | 3 (20.0) | 1 (8.3) | 10 (23.8) |
| Nasopharyngitis | 5 (33.3) | 5 (33.3) | 1 (8.3) | 11 (26.2) |
| Headache | 5 (33.3) | 1 (6.7) | 1 (8.3) | 7 (16.7) |
| Fatigue | 4 (26.7) | 4 (26.7) | 1 (8.3) | 9 (21.4) |
| Diarrhea | 4 (26.7) | 3 (20.0) | 4 (33.3) | 11 (26.2) |
| Pyrexia | 4 (26.7) | 2 (13.3) | 3 (25.0) | 9 (21.4) |
| Pain in extremity | 4 (26.7) | 1 (6.7) | 3 (25.0) | 8 (19.0) |
| Dizziness | 4 (26.7) | 1 (6.7) | 0 | 5 (11.9) |
| Arthralgia | 3 (20.0) | 4 (26.7) | 3 (25.0) | 10 (23.8) |
| Cough | 3 (20.0) | 4 (26.7) | 0 | 7 (16.7) |
| Erythema | 2 (13.3) | 4 (26.7) | 0 | 6 (14.3) |
| Asthenia | 1 (6.7) | 2 (13.3) | 4 (33.3) | 7 (16.7) |
| Peripheral edema | 1 (6.7) | 0 | 4 (33.3) | 5 (11.9) |
| Muscle spasms | 1 (6.7) | 0 | 3 (25.0) | 4 (9.5) |
| Most common (≥5%) grade ≥3 | | | | |
| TEAEs, n (%) | | | | |
| Hematologic | | | | |
| Anemia | 1 (6.7) | 1 (6.7) | 2 (16.7) | 4 (9.5) |
| Lymphopenia | 2 (13.3) | 0 | 1 (8.3) | 3 (7.1) |
| Neutropenia | 0 | 3 (20.0) | 0 | 3 (7.1) |
| Nonhematologic | | | | |
| Bone pain | 1 (6.7) | 1 (6.7) | 1 (8.3) | 3 (7.1) |

TEAE, treatment-emergent adverse event.

TABLE 4
Most Common (At Least 10%) Treatment-emergent Adverse Events by
System Organ Class and Preferred Term; All Treated Analysis Set

| | Part 3 (1800 mg CF) | | | |
|---|---|---|---|---|
| | 3-wk taper | 2-wk taper | 1-wk taper | Total |
| Analysis set: all treated | 15 | 15 | 12 | 42 |
| Total number of subjects with TEAE | 15 (100.0%) | 15 (100.0%) | 12 (100.0%) | 42 (100.0%) |
| MedDRA system organ class/Preferred term | | | | |
| General disorders and administration site | 11 (73.3%) | 10 (66.7%) | 9 (75.0%) | 30 (71.4%) |
| conditions | | | | |
| Fatigue | 4 (26.7%) | 4 (26.7%) | 1 (8.3%) | 9 (21.4%) |
| Pyrexia | 4 (26.7%) | 2 (13.3%) | 3 (25.0%) | 9 (21.4%) |
| Asthenia | 1 (6.7%) | 2 (13.3%) | 4 (33.3%) | 7 (16.7%) |
| Chills | 2 (13.3%) | 1 (6.7%) | 2 (16.7%) | 5 (11.9%) |
| Edema peripheral | 1 (6.7%) | 0 | 4 (33.3%) | 5 (11.9%) |
| Infections and infestations | 12 (80.0%) | 9 (60.0%) | 6 (50.0%) | 27 (64.3%) |
| Nasopharyngitis | 5 (33.3%) | 5 (33.3%) | 1 (8.3%) | 11 (26.2%) |
| Upper respiratory tract infection | 6 (40.0%) | 3 (20.0%) | 1 (8.3%) | 10 (23.8%) |
| Gastrointestinal disorders | 9 (60.0%) | 7 (46.7%) | 8 (66.7%) | 24 (57.1%) |
| Nausea | 8 (53.3%) | 3 (20.0%) | 2 (16.7%) | 13 (31.0%) |
| Diarrhea | 4 (26.7%) | 3 (20.0%) | 4 (33.3%) | 11 (26.2%) |
| Constipation | 2 (13.3%) | 3 (20.0%) | 2 (16.7%) | 7 (16.7%) |
| Vomiting | 2 (13.3%) | 2 (13.3%) | 2 (16.7%) | 6 (14.3%) |
| Musculoskeletal and connective tissue disorders | 6 (40.0%) | 9 (60.0%) | 8 (66.7%) | 23 (54.8%) |
| Arthralgia | 3 (20.0%) | 4 (26.7%) | 3 (25.0%) | 10 (23.8%) |
| Pain in extremity | 4 (26.7%) | 1 (6.7%) | 3 (25.0%) | 8 (19.0%) |
| Bone pain | 2 (13.3%) | 3 (20.0%) | 2 (16.7%) | 7 (16.7%) |
| Back pain | 1 (6.7%) | 3 (20.0%) | 2 (16.7%) | 6 (14.3%) |
| Respiratory, thoracic and mediastinal disorders | 6 (40.0%) | 8 (53.3%) | 1 (8.3%) | 15 (35.7%) |
| Cough | 3 (20.0%) | 4 (26.7%) | 0 | 7 (16.7%) |
| Oropharyngeal pain | 2 (13.3%) | 3 (20.0%) | 0 | 5 (11.9%) |
| Nervous system disorders | 7 (46.7%) | 4 (26.7%) | 2 (16.7%) | 13 (31.0%) |
| Headache | 5 (33.3%) | 1 (6.7%) | 1 (8.3%) | 7 (16.7%) |
| Dizziness | 4 (26.7%) | 1 (6.7%) | 0 | 5 (11.9%) |
| Metabolism and nutrition disorders | 7 (46.7%) | 4 (26.7%) | 1 (8.3%) | 12 (28.6%) |
| Decreased appetite | 3 (20.0%) | 1 (6.7%) | 1 (8.3%) | 5 (11.9%) |
| Blood and lymphatic system disorders | 4 (26.7%) | 3 (20.0%) | 4 (33.3%) | 11 (26.2%) |
| Anaemia | 1 (6.7%) | 2 (13.3%) | 4 (33.3%) | 7 (16.7%) |
| Skin and subcutaneous tissue disorders | 6 (40.0%) | 5 (33.3%) | 0 | 11 (26.2%) |
| Erythema | 2 (13.3%) | 4 (26.7%) | 0 | 6 (14.3%) |

Keys: CF = Co-Formulated.
Adverse events are reported using MedDRA version 23.1.
Percentages are calculated with the number of subjects in each group as denominator.

TABLE 5
Most Common (At Least 5%) Treatment-emergent Grade
3 or Higher Adverse Events by System Organ Class
and Preferred Term; All Treated Analysis Set

| | Part 3 (1800 mg CF) | | | |
|---|---|---|---|---|
| | 3 wk taper | 2 wk taper | 1 wk taper | Total |
| Analysis set: all | 15 | 15 | 12 | 42 |
| treated | | | | |
| Total number of | 9 (60.0%) | 8 (53.3%) | 4 (33.3%) | 21 (50.0%) |
| subjects with grade 3 | | | | |
| or higher TEAE | | | | |
| MedDRA system | | | | |
| organ class/ | | | | |
| Preferred term | | | | |
| Blood and | 3 (20.0%) | 3 (20.0%) | 2 (16.7%) | 8 (19.0%) |
| lymphatic system | | | | |
| disorders | | | | |
| Anaemia | 1 (6.7%) | 1 (6.7%) | 2 (16.7%) | 4 (9.5%) |
| Lymphopenia | 2 (13.3%) | 0 | 1 (8.3%) | 3 (7.1%) |
| Neutropenia | 0 | 3 (20.0%) | 0 | 3 (7.1%) |
| Musculoskeletal | 2 (13.3%) | 1 (6.7%) | 1 (8.3%) | 4 (9.5%) |
| and connective | | | | |
| tissue disorders | | | | |
| Bone pain | 1 (6.7%) | 1 (6.7%) | 1 (8.3%) | 3 (7.1%) |

Keys: CF = Co-Formulated.
Adverse events are reported using MedDRA version 23.1.
Percentages are calculated with the number of subjects in each group as denominator.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

### Embodiments

1. A method of treating a hematologic malignancy, comprising administering to a subject in need thereof a therapeutically effective amount of an anti-CD38 antibody and a corticosteroid for a time sufficient to treat the hematologic malignancy, wherein the dosing regimen includes a reduction, elimination, or reduction followed by elimination, of corticosteroid administration to the subject.
2. The method of Embodiment 1, wherein the corticosteroid administered to the subject is reduced by about 60% and then eliminated during a 28-day treatment cycle.
3. The method of Embodiment 1, wherein the corticosteroid administered to the subject is reduced by about 60% and then by about 30% and then eliminated during a 28-day treatment cycle.
4. The method of any one of Embodiments 1-3, wherein the corticosteroid administered to the subject is administered once and then eliminated during a 28-day treatment cycle.
5. The method of any one of Embodiments 1-4, wherein the anti-CD38 antibody is administered once weekly, every 2 weeks, or every 4 weeks during a 28-day cycle.
6. The method of Embodiments 1-4, wherein the anti-CD38 antibody is administered once weekly during Cycle 1, every 2 weeks during Cycles 2-5, and every 4 weeks thereafter.
7 The method of Embodiment 1, comprising administering to the subject a therapy on a 28-day cycle, wherein the therapy comprises:

administering about 1,800 mg anti-CD38 antibody and about 30,000 U hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8;

administering about 20 mg post-dose corticosteroid on day 8;

administering about 30 mg pre-dose corticosteroid on day 15; and

administering about 20 mg post-dose corticosteroid on day 15, of the 28-day cycle.
8. The method of Embodiment 1, comprising administering to the subject a therapy on a 28-day cycle, wherein the therapy comprises:

administering about 1,800 mg daratumumab and about 30,000 U rHuPH20 recombinant hyaluronidase subcutaneously on days 1, 8, 15 and 22;

administering about 100 mg pre-dose methylprednisolone (MP) orally or intravenously on day 1;

administering about 20 mg post-dose MP orally on days 1 and 2;

administering about 60 mg pre-dose MP orally or intravenously on day 8;

administering about 20 mg post-dose MP orally on day 8;

administering about 30 mg pre-dose MP orally or intravenously on day 15; and

administering about 20 mg post-dose MP orally on day 15, of the 28-day cycle.
9. The method of Embodiment 1, comprising administering to the subject a therapy on a 28-day cycle, wherein the therapy comprises:

administering about 1,800 mg anti-CD38 antibody and about 30,000 U rHuPH20 hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8; and

administering about 20 mg post-dose corticosteroid on day 8, of the 28-day cycle.
10. The method of Embodiment 1, comprising administering to the subject a therapy on a 28-day cycle, wherein the therapy comprises:

administering about 1,800 mg daratumumab and about 30,000 U rHuPH20 recombinant hyaluronidase subcutaneously on days 1, 8, 15 and 22;

administering about 100 mg pre-dose methylprednisolone (MP) orally or intravenously on day 1;

administering about 20 mg post-dose MP orally on days 1 and 2;

administering about 60 mg pre-dose MP orally or intravenously on day 8; and

administering about 20 mg post-dose MP orally on day 8, of the 28-day cycle.
11. The method of Embodiment 1, comprising administering to the subject a therapy on a 28-day cycle, wherein the therapy comprises:

administering about 1,800 mg anti-CD38 antibody and about 30,000 U hyaluronidase subcutaneously on days 1, 8, 15 and 22; and

administering about 20 mg pre-dose corticosteroid intravenously on day 1, of the 28-day cycle.
12. The method of Embodiment 1, comprising administering to the subject a therapy on a 28-day cycle, wherein the therapy comprises:
   a) administering about 1,800 mg of daratumumab and about 30,000 U rHuPH20 recombinant hyaluronidase subcutaneously on days 1, 8, 15 and 22; and
   b) administering about 20 mg pre-dose dexamethasone intravenously on day 1, of the 28-day cycle.
13. A method of treating hematologic malignancy to a subject in need thereof, comprising administering to the subject a therapy on a 28-day cycle, wherein the therapy comprises:

administering about 1,800 mg anti-CD38 antibody and about 30,000 U hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8;

administering about 20 mg post-dose corticosteroid on day 8;

administering about 30 mg pre-dose corticosteroid on day 15; and

administering about 20 mg post-dose corticosteroid on day 15, of the 28-day cycle.
14. A method of treating hematologic malignancy to a subject in need thereof, comprising administering to the subject a therapy on a 28-day cycle, wherein the therapy comprises:

administering about 1,800 mg anti-CD38 antibody and about 30,000 U rHuPH20 hyaluronidase on days 1, 8, 15 and 22;

administering about 100 mg pre-dose corticosteroid on day 1;

administering about 20 mg post-dose corticosteroid on days 1 and 2;

administering about 60 mg pre-dose corticosteroid on day 8; and

administering about 20 mg post-dose corticosteroid on day 8, of the 28-day cycle.
15. A method of treating hematologic malignancy in a subject in need thereof, the method comprising administering to the subject a therapy on a 28-day cycle, wherein the therapy comprises:
   a) administering about 1,800 mg of the anti-CD38 antibody and about 30,000 U on days 1, 8, 15 and 22; and
   b) administering about 20 mg pre-dose corticosteroid on day 1, of the 28-day cycle.
16. A method of treating a hematologic malignancy, comprising administering to a subject in need thereof a therapeutically effective amount of an anti-CD38 antibody and a corticosteroid dose of <0.05 mg/kg/day or equivalent for a time sufficient to treat the hematologic malignancy.
17. The method of Embodiment 16, wherein a corticosteroid dose of <0.01 mg/kg/day or equivalent is administered.
18. A method of treating a hematologic malignancy, comprising administering to a subject in need thereof a therapeutically effective amount of an anti-CD38 antibody for a time sufficient to treat the hematologic malignancy, wherein disease control or complete remission is achieved and/or maintained at a corticosteroid dose of ≤0.05 mg/kg/day or equivalent.
19. The method of Embodiment 18, wherein the disease control or complete remission is achieved and/or maintained at a corticosteroid dose of ≤0.01 mg/kg/day or equivalent.
20. The method of Embodiment 18, wherein the disease control or complete remission is achieved and/or maintained without co-administering a corticosteroid.
21. The method of any one of Embodiments 16-20, further comprising administering to the subject a prior therapy on a 28-day cycle, wherein the prior therapy comprises:

administering about 1,800 mg daratumumab and about 30,000 U rHuPH20 recombinant hyaluronidase subcutaneously on days 1, 8, 15 and 22;

administering about 100 mg pre-dose methylprednisolone (MP) orally or intravenously on day 1;

administering about 20 mg post-dose MP orally on days 1 and 2;

administering about 60 mg pre-dose MP orally or intravenously on day 8;

administering about 20 mg post-dose MP orally on day 8;

administering about 30 mg pre-dose MP orally or intravenously on day 15; and

administering about 20 mg post-dose MP orally on day 15, of the 28-day cycle.
22. The method of any one of Embodiments 16-20, further comprising administering to the subject a prior therapy on a 28-day cycle, wherein the prior therapy comprises:

administering about 1,800 mg daratumumab and about 30,000 U rHuPH20 recombinant hyaluronidase subcutaneously on days 1, 8, 15 and 22;

administering about 100 mg pre-dose methylprednisolone (MP) orally or intravenously on day 1;

administering about 20 mg post-dose MP orally on days 1 and 2;

administering about 60 mg pre-dose MP orally or intravenously on day 8; and

administering about 20 mg post-dose MP orally on day 8, of the 28-day cycle.
23. The method of any one of Embodiments 16-20, further comprising administering to the subject a prior therapy on a 28-day cycle, wherein the prior therapy comprises:
   a) administering about 1,800 mg daratumumab and about 30,000 U rHuPH20 recombinant hyaluronidase subcutaneously on days 1, 8, 15 and 22; and
   b) administering about 20 mg pre-dose dexamethasone intravenously on day 1, of the 28-day cycle.
24. The method of any one of Embodiments 1-23, wherein the corticosteroid comprises bethamethasone, cortisol, cortisone, dexamethasone, glucocorticoid, hydrocortisone, methylprednisolone (MP), prednisolone, prednisone, triamcinolone, or a combination thereof.
25. The method of any one of Embodiments 1-23, wherein the corticosteroid comprises dexamethasone, methylprednisolone, prednisone, or a combination thereof.
26. The method of any one of Embodiments 1-25, wherein the hematologic malignancy is a CD38-positive hematologic malignancy.
27. The method of any one of Embodiments 1-25, wherein the hematological malignancy is multiple myeloma.
28. The method of Embodiment 27, wherein the multiple myeloma is relapsed or refractory multiple myeloma.
29. The method of any one of Embodiments 1-28, wherein the anti-CD38 antibody comprises:
   a) a heavy chain complementarity determining region 1 (HCDR1), HCDR2 and HCDR3 amino acid sequences of SEQ ID NOs:6, 7 and 8, respectively; and/or
   b) a light chain complementarity determining region 1 (LCDR1), LCDR2 and LCDR3 amino acid sequences of SEQ ID NOs:9, 10 and 11, respectively.
30. The method of Embodiment 29, wherein the anti-CD38 antibody comprises a heavy chain variable region (VH) sequence of SEQ ID NO:4, a light chain variable region (VL) sequence of SEQ ID NO:5, or both.
31. The method of Embodiment 29, wherein the anti-CD38 antibody comprises a heavy chain sequence of SEQ ID NO:12, a light chain sequence of SEQ ID NO:13, or both.
32. The method of any one of Embodiments 1-31, wherein the anti-CD38 antibody is of the IgG1, IgG2, IgG3 or IgG4 subtype.
33. The method of Embodiment 32, wherein the anti-CD38 antibody is of the IgG1 subtype.
34. The method of Embodiment 33, wherein the anti-CD38 antibody is of the IgG1/κ subtype.
35. The method of any one of Embodiments 1-28, wherein the anti-CD38 antibody is daratumumab.
36. The method of any one of Embodiments 1-35, wherein the anti-CD38 antibody is administered in a pharmaceutical composition comprising from about 1,200 mg to about 5,000 mg of the anti-CD38 antibody.
37. The method of Embodiment 36, wherein the pharmaceutical composition comprises about 1,800 mg of the anti-CD38 antibody.
38. The method of Embodiment 36 or 37, wherein the pharmaceutical composition further comprises a hyaluronidase.
39. The method of Embodiment 38, wherein the hyaluronidase is rHuPH20 recombinant hyaluronidase.
40. The method of Embodiment 38 or 39, wherein the pharmaceutical composition comprises from about 750 U to about 75,000 U of the hyaluronidase.
41. The method of Embodiment 40, wherein the pharmaceutical composition comprises about 30,000 U of the hyaluronidase.
42. The method of any one of Embodiments 36-41, wherein the anti-CD38 antibody and the hyaluronidase are administered in a co-formulation.
43. The method of any one of Embodiments 36-42, wherein the pharmaceutical composition further comprises:

about 4.9 mg L-histidine;

about 18.4 mg L-histidine hydrochloride monohydrate;

about 13.5 mg L-methionine;

about 6 mg polysorbate 20 (PS-20); and

about 735.1 mg sorbitol.
44. The method of any one of Embodiments 36-43, wherein the pharmaceutical composition has a pH of about pH 5.5.
45. The method of any one of Embodiments 36-43, wherein the pharmaceutical composition has a pH of about pH 5.6.
46. The method of any one of Embodiments 36-45, wherein the pharmaceutical composition has a total volume of about 15 mL.
47. The method of any one of Embodiments 1-46, wherein the anti-CD38 antibody is administered subcutaneously.
48. The method of any one of Embodiments 1-47, wherein the subject is 18 years of age or older.
49. The method of any one of Embodiments 1-48, wherein the subject is naive to anti-CD38 therapy.
50. The method of any one of Embodiments 1-48, wherein the subject has received at least two prior lines of anti-myeloma therapy.
51. The method of Embodiment 50, wherein the at least two prior lines of anti-myeloma therapy comprise a proteasome inhibitor (PI), administering an immunomodulatory drug (IMiD), hematopoietic stem cell transplantation (HSCT), a maintenance therapy, or a combination thereof.
52. The method of Embodiment 51, wherein the IMid is lenalidomide
53. The method of Embodiment 51 or 52, wherein the PI is bortezomib, carfilzomib, or ixazomib.
54. The method of any one of Embodiments 51-53, wherein the HSCT is an autologous HSCT.
55. The method of any one of Embodiments 51-53, wherein the two lines of therapy comprise am IMid and a PI.
56. The method of any one of Embodiments 1-55, wherein the subject is refractory to at least one line of therapy.
57. The method of any one of Embodiments 1-56, wherein the method elicits at least a partial response in the subject.
58. The method of Embodiment 57, wherein the method elicits a partial response in the subject.
59. The method of any one of Embodiments 1-56, wherein the method elicits at least a very good partial response in the subject.
60. The method of Embodiment 59, wherein the method elicits a complete response in the subject.
61. The method of Embodiment 59, wherein the method elicits a stringent complete response in the subject.
62. The method of any one of Embodiments 1-61, wherein the method improves one or more outcome measurements of the subject.
63. The method of Embodiment 62, wherein the one or more outcome measurements comprise progression-free survival, duration of response, or at least partial response, or any combination thereof.
64. The method of Embodiment 62, wherein the one or more outcome measures comprise a partial response, a very good partial response, a complete response, or a stringent complete response.
65. The method of any one of Embodiments 1-61, wherein the subject experiences an improvement in one or more outcome measures consistent with a subject receiving anti-CD38 antibody administration and continuous corticosteroid administration.
66. The method of any one of Embodiments 1-61, wherein the subject experiences an increased improvement in one or more outcome measures compared with a subject receiving anti-CD38 antibody administration and continuous corticosteroid administration.
67. The method of any one of Embodiments 1-66, further comprising administering to the subject one or more additional therapeutic agents.
68. The method of Embodiment 67, wherein one or more additional therapeutic agents comprise a T cell expressing chimeric antigen receptor (CAR) (CAR-T cell), a natural killer cell expressing CAR (CAR-NK cell), a macrophage expressing CAR (CAR-M cell), a chemotherapeutic agent, a bispecific antibody, an immune checkpoint inhibitor, a T-cell redirector, or a combination thereof.
69. The method of Embodiment 68, wherein the CAR-T cell, the CAR-NK cell, or the CAR-M cell is allogeneic.
70. The method of Embodiment 68 or 69, wherein the CAR comprises an extracellular antigen-binding domain, a transmembrane domain and an intracellular signaling domain.
71. The method of Embodiment 70, wherein the intracellular signaling domain comprises a T-cell surface glycoprotein CD3 zeta chain component
72. The method of Embodiment 70 or 71, wherein the extracellular antigen-binding domain binds G-protein coupled receptor family C group 5 member D (GPRC5D).
73. The method of Embodiment 72, wherein the extracellular antigen-binding domain binds GPRC5D and CD3.
74. The method of Embodiment 72 or 73, wherein the one or more additional therapeutic agents comprise an anti-GPRC5D CAR-T, an anti-GPRC5D CAR-NK or a combination thereof.
75. The method of Embodiment 70, wherein the extracellular antigen-binding domain binds B cell maturation antigen (BCMA).
76. The method of Embodiment 75, wherein the extracellular antigen-binding domain binds BCMA and CD3.
77. The method of Embodiment 75 or 76, wherein the one or more additional therapeutic agents comprise an anti-BCMA CAR-T, an anti-BCMA CAR-NK, or a combination thereof.
78. The method of any one of Embodiments 68-77, wherein the immune checkpoint inhibitor comprises an anti-PD-1 antibody, an anti-PD-L1 antibody, an anti-PD-L2 antibody, an anti-LAG3 antibody, an anti-TIM3 antibody, an anti-CTLA-4 antibody, or a combination thereof.
79. The method of any one of Embodiments 68-78, wherein the T-cell redirector comprises a soluble bispecific antibody (bsAb) or a membrane-anchored chimeric antigen receptor, or a combination thereof.
80. The method of Embodiment 79, wherein the soluble bispecific antibody binds GPRC5D and CD3.
81. The method of Embodiment 79, wherein the soluble bispecific antibody binds BCMA and CD3.

What is claimed is:

1. A method of treating a hematologic malignancy, comprising administering to a subject in need thereof a therapeutically effective amount of an anti-CD38 antibody and a corticosteroid for a time sufficient to treat the hematologic malignancy, wherein the dosing regimen includes a reduction, elimination, or reduction followed by elimination, of corticosteroid administration to the subject.

2. The method of claim 1, wherein the corticosteroid administered to the subject is:
a) reduced by about 60% and then eliminated during a 28-day treatment cycle;
b) reduced by about 60% and then by about 30% and then eliminated during a 28-day treatment cycle; or
c) administered once and then eliminated during a 28-day treatment cycle.

3. The method of claim 1, wherein the anti-CD38 antibody is administered once weekly, every 2 weeks, or every 4 weeks during a 28-day cycle.

4. The method of claim 3, wherein the anti-CD38 antibody is administered once weekly during Cycle 1, every 2 weeks during Cycles 2-5, and every 4 weeks thereafter.

5. The method of claim 1, comprising administering to the subject a therapy on a 28-day cycle, wherein the therapy comprises:
administering about 1,800 mg anti-CD38 antibody and about 30,000 U hyaluronidase on days 1, 8, 15 and 22;
administering about 100 mg pre-dose corticosteroid on day 1;
administering about 20 mg post-dose corticosteroid on days 1 and 2;
administering about 60 mg pre-dose corticosteroid on day 8;
administering about 20 mg post-dose corticosteroid on day 8;
administering about 30 mg pre-dose corticosteroid on day 15; and
administering about 20 mg post-dose corticosteroid on day 15, of the 28-day cycle.

6. The method of claim 5, wherein the therapy comprises:
administering about 1,800 mg daratumumab and about 30,000 U rHuPH20 recombinant hyaluronidase subcutaneously on days 1, 8, 15 and 22;
administering about 100 mg pre-dose methylprednisolone (MP) orally or intravenously on day 1;
administering about 20 mg post-dose MP orally on days 1 and 2;
administering about 60 mg pre-dose MP orally or intravenously on day 8;
administering about 20 mg post-dose MP orally on day 8;
administering about 30 mg pre-dose MP orally or intravenously on day 15; and
administering about 20 mg post-dose MP orally on day 15, of the 28-day cycle.

7. The method of claim 1, comprising administering to the subject a therapy on a 28-day cycle, wherein the therapy comprises:
administering about 1,800 mg anti-CD38 antibody and about 30,000 U rHuPH20 hyaluronidase on days 1, 8, 15 and 22;
administering about 100 mg pre-dose corticosteroid on day 1;
administering about 20 mg post-dose corticosteroid on days 1 and 2;
administering about 60 mg pre-dose corticosteroid on day 8; and
administering about 20 mg post-dose corticosteroid on day 8, of the 28-day cycle.

8. The method of claim 7, wherein the therapy comprises:
administering about 1,800 mg daratumumab and about 30,000 U rHuPH20 recombinant hyaluronidase subcutaneously on days 1, 8, 15 and 22;
administering about 100 mg pre-dose methylprednisolone (MP) orally or intravenously on day 1;
administering about 20 mg post-dose MP orally on days 1 and 2;
administering about 60 mg pre-dose MP orally or intravenously on day 8; and
administering about 20 mg post-dose MP orally on day 8, of the 28-day cycle.

9. The method of claim 1, comprising administering to the subject a therapy on a 28-day cycle, wherein the therapy comprises:
administering about 1,800 mg anti-CD38 antibody and about 30,000 U hyaluronidase subcutaneously on days 1, 8, 15 and 22; and
administering about 20 mg pre-dose corticosteroid intravenously on day 1, of the 28-day cycle.

10. The method of claim 9, wherein the therapy comprises:
a) administering about 1,800 mg of daratumumab and about 30,000 U rHuPH20 recombinant hyaluronidase subcutaneously on days 1, 8, 15 and 22; and
b) administering about 20 mg pre-dose dexamethasone intravenously on day 1, of the 28-day cycle.

11. The method of claim 1, wherein the corticosteroid comprises bethamethasone, cortisol, cortisone, dexamethasone, glucocorticoid, hydrocortisone, methylprednisolone (MP), prednisolone, prednisone, triamcinolone, or a combination thereof.

12. The method of claim 11, wherein the corticosteroid comprises dexamethasone, methylprednisolone, prednisone, or a combination thereof.

13. The method of claim 1, wherein the hematologic malignancy is a CD38-positive hematologic malignancy.

14. The method of claim 1, wherein the anti-CD38 antibody comprises:
a) a heavy chain complementarity determining region 1 (HCDR1), HCDR2 and HCDR3 amino acid sequences of SEQ ID NOs:6, 7 and 8, respectively, and a light chain complementarity determining region 1 (LCDR1), LCDR2 and LCDR3 amino acid sequences of SEQ ID NOs:9, 10 and 11, respectively;
b) a heavy chain variable region (VH) sequence of SEQ ID NO:4, and a light chain variable region (VL) sequence of SEQ ID NO:5; or
c) a heavy chain sequence of SEQ ID NO:12, and a light chain sequence of SEQ ID NO:13, or
a combination of the foregoing.

15. A method of treating a hematologic malignancy, comprising administering to a subject in need thereof a therapeutically effective amount of an anti-CD38 antibody and a corticosteroid dose of <0.05 mg/kg/day or equivalent for a time sufficient to treat the hematologic malignancy.

16. The method of claim 15, wherein a corticosteroid dose of <0.01 mg/kg/day or equivalent is administered.

17. The method of claim 15, wherein the corticosteroid comprises bethamethasone, cortisol, cortisone, dexamethasone, glucocorticoid, hydrocortisone, methylprednisolone (MP), prednisolone, prednisone, triamcinolone, or a combination thereof.

18. A method of treating a hematologic malignancy, comprising administering to a subject in need thereof a therapeutically effective amount of an anti-CD38 antibody for a time sufficient to treat the hematologic malignancy, wherein disease control or complete remission is achieved and/or maintained at a corticosteroid dose of ≤0.05 mg/kg/day or equivalent.

19. The method of claim 18, wherein the disease control or complete remission is achieved and/or maintained at a corticosteroid dose of ≤0.01 mg/kg/day or equivalent.

20. The method of claim 18, wherein the disease control or complete remission is achieved and/or maintained without co-administering a corticosteroid.
